(19) United States
(12) Patent Application Publication
(10) Pub. No.: US 2007/0219178 A1
(43) Pub. Date: Sep. 20, 2007

(54) PREVENTIVE OR THERAPEUTIC AGENTS FOR MULTIPLE SCLEROSIS

(75) Inventor: Kenzo Muramoto, Tsukuba-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eisai Co., Ltd., Bunkyo-ku (JP)

(21) Appl. No.: 10/596,212

(22) PCT Filed: Oct. 7, 2004
(86) PCT No.: PCT/JP04/14857

§ 371 Date: Jan. 12, 2007

(30) Foreign Application Priority Data
Dec. 4, 2003 (JP) 2003-405337

(51) Int. Cl. A61K 31/551; A61K 31/522; A61K 31/55; A61K 31/519

(52) U.S. Cl. 514/210.21; 514/218; 514/217.06; 514/252.16; 514/263.2; 514/263.22; 514/262.1

(57) ABSTRACT

The preventive or therapeutic agents of the present invention for multiple sclerosis comprise
compounds represented by the following formula (I), or salts or hydrates thereof, 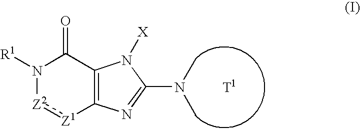
[wherein, T¹, X, Z¹, Z², and R¹ have the same meaning as T¹, X, Z¹, Z², and R¹ in this application].

### TECHNICAL FIELD

The present invention relates to preventive or therapeutic agents for multiple sclerosis comprising condensed imidazole derivatives.

### BACKGROUND ART

Multiple sclerosis (MS) is a cryptogenic demyelinating disease in the central nervous system, which most commonly affects young adults. Since it produces multifocal demyelinating lesions in central nervous tissues, such as the brain, spinal cord and optic nerve, the various neurological symptoms occur in cycles of relapse and remission. Damage can occur anywhere in the central nerve, and clinical symptoms include visual disturbances caused by disorders of the optic nerve and spinal cord, motor paralysis, gait disturbance, numbness, paresthesia, sensory paralysis, and ophthalmalgia. Antibodies against a basic protein (myelin basic protein: MBP), galactocerebroside, ganglioside, and such, which are myelin components, are found to be increased in patients' sera and cerebrospinal fluid. Some observations also suggest that autoimmune mechanisms such as lymphocyte invasion of lesions are involved in cellular immunity, but this is not definitive.

Dipeptidyl peptidase-IV (DPPIV (CD26)) is a serine protease that specifically hydrolytically cleaves the dipeptide-X-Pro (where X may represent any amino acid) from the free N terminus of a polypeptide chain.

Experimental autoimmune encephalomyelitis (EAE) is an animal model for MS that has been accepted for a number of years (Non-patent Document 1). Steinbrecher et al. have reported a response when low-molecular-weight compound 140 (Lys[Z(NO)₂]-pyrrolidide) (M=414.89), a compound with DPPIV inhibitory action, is subcutaneously administered to the EAE model (Non-patent Document 2).

[Non-patent Document 1] Chn. Immunol. Immunopath. 77:4-13 (1995)

[Non-patent Document 2] The Journal of Immunology, 2001, 116, p 2041-2048

### DISCLOSURE OF THE INVENTION

The present inventors conducted dedicated studies in view of the above background. As a result, the inventors found that condensed imidazole derivatives, including hypoxanthine derivatives, imidazopyridazinone derivatives and xanthine derivatives, could be used as superior preventive or therapeutic agents for multiple sclerosis. Thus, the inventors completed the present invention. Specifically, the present invention comprises:
[1] a preventive or therapeutic agent for multiple sclerosis, which comprises the compound represented by formula (I), or a salt or hydrate thereof, 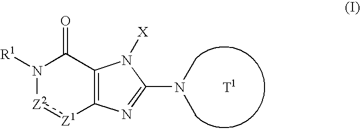
[wherein,
T¹ represents a mono- or bicyclic 4- to 12-membered heterocyclic group comprising one or two nitrogen atoms in a ring, which may have substituents;
X represents a C₁₋₆ alkyl group that may have a substituent, a C₂₋₆ alkenyl group that may have a substituent, a C₂₋₆ alkynyl group that may have a substituent, a C₆₋₁₀ aryl group that may have a substituent, a 5- to 10-membered heteroaryl group that may have a substituent, a C₆₋₁₀ aryl C₁₋₆ alkyl group that may have a substituent, or a 5- to 10-membered heteroaryl C₁₋₆ alkyl group that may have a substituent;
in formula (I), the following formula 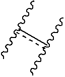
represents a single or double bond;
and when the formula 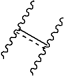
represents a single bond, Z¹ represents a group represented by the formula —NR²—, and Z² represents a carbonyl group;
when the formula 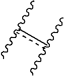
represents a double bond, Z¹ and Z² each independently represent a nitrogen atom or a group represented by the formula —CR²═;
R¹ and R² each independently represent a group represented by the formula -A⁰-A¹-A²
(wherein, A⁰ represents a single bond or a C₁₋₆ alkylene group that may have one to three groups selected from a substituent group B described below;
A¹ represents a single bond, an oxygen atom, a sulfur atom, a sulfinyl group, a sulfonyl group, a carbonyl group, a formula —O—CO—, a formula —CO—O—, a formula —NR^{A}—, a formula —CO—NR^{A}—, a formula —NR^{A}—CO—, a formula —SO₂—NR^{A}—, or a formula —NR^{A}—SO₂—;
A² and R^{A} each independently represent a hydrogen atom, a halogen atom, a cyano group, a guanidino group, a C₁₋₆ alkyl group, a C₃₋₈ cycloalkyl group, a C₃₋₈ cycloalkenyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, a C₆₋₁₀ aryl C₁₋₆ alkyl group, or a C₂₋₇ alkyl carbonyl group;
with the proviso that A² and R^{A} may each independently have one to three groups selected from substituent group B described below);
   when Z² represents the formula —CR²═, R¹ and R² may together form a 5- to 7-membered ring;
   <Substituent group B>
   substituent group B refers to a group consisting of:
   a hydroxyl group, a mercapto group, a cyano group, a nitro group, a halogen atom, a trifluoromethyl group, a trifluoromethoxy group, an alkylenedioxy group, a C₁₋₆ alkyl group that may have a substituent, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a C₁₋₆ alkoxy group, a C₁₋₆ alkylthio group, groups represented by the formulae —SO₂—NR^{B1}—R^{B2}, —NR^{B1}—CO—R^{B2}, and —NR^{B1}—R^{B2} (where R^{B1} and R^{B2} each independently represent a hydrogen atom or a C₁₋₆ alkyl group), a group represented by the formula —CO—R^{B3} (where R^{B3} represents a 4- to 8-membered heterocyclic group), and groups represented by the formulae —CO—R^{B4}—R^{B5} and —CH₂—CO—R^{B4}—R^{B5} (where R^{B4} represents a single bond, an oxygen atom, or a formula —NR^{B6}—; R^{B5} and R^{B6} each independently represent a hydrogen atom, a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic C₁₋₆ alkyl group, a C₆₋₁₀ aryl C₁₋₆ alkyl group, or a 5-10-membered heteroaryl C₁₋₆ alkyl group)];
   [2] a preventive or therapeutic agent for multiple sclerosis, which comprises the compound represented by formula (II), or a salt or hydrate thereof, 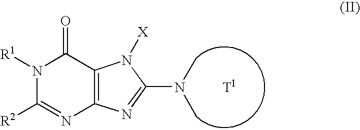
   [wherein, X, R¹, R² and T¹ have the same meaning as X, R¹, R² and T¹ of [1]];
   [3] a preventive or therapeutic agent for multiple sclerosis, which comprises the compound represented by formula (III), or a salt or hydrate thereof, 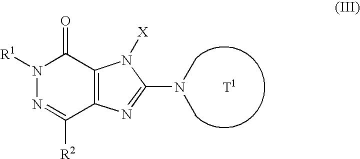
   [wherein, X, R¹, R² and T¹ have the same meaning as X, R¹, R² and T¹ of [1]];
   [4] a preventive or therapeutic agent for multiple sclerosis, which comprises the compound represented by formula (IV), or a salt or hydrate thereof, 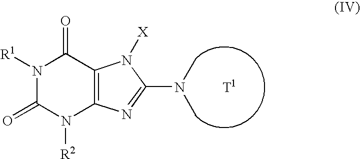
   [wherein, X, R¹, R² and T¹ have the same meaning as X, R¹, R² and T¹ of [1]];
   [5] a preventive or therapeutic agent for multiple sclerosis, which comprises the compound of any one of [1] to [4], or a salt or hydrate thereof, wherein T¹ described above is a group represented by the following formula: 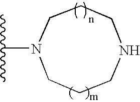
   (where n and m each independently represent zero or one), an azetidin-1-yl group that may have a substituent, a pyrrolidine-1-yl group that may have a substituent, a piperidine-1-yl group that may have a substituent, or an azepan-1-yl group that may have a substituent;
   [6] a preventive or therapeutic agent for multiple sclerosis, which comprises the compound of any one of [1] to [4], or a salt or hydrate thereof, wherein T¹ described above is a group represented by the following formula: 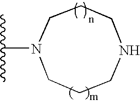
   (where n and m each independently represent zero or one), an azetidin-1-yl group that may have an amino group, a pyrrolidin-1-yl group that may have an amino group, a piperidin-1-yl group that may have an amino group, or an azepan-1-yl group that may have an amino group;
   [7] a preventive or therapeutic agent for multiple sclerosis, which comprises the compound of any one of [1] to [4], or a salt or hydrate thereof, wherein T¹ described above is a piperazine-1-yl group or a 3-aminopiperidine-1-yl group;
   [8] a preventive or therapeutic agent for multiple sclerosis, which comprises the compound of any one of [1] to [4], or a salt or hydrate thereof, wherein T¹ described above is a piperazine-1-yl group;
   [9] a preventive or therapeutic agent for multiple sclerosis, which comprises the compound according to any one of [1] to [8], or a salt or hydrate thereof, wherein X described above is a group represented by the formula —X¹—X² (where X¹ represents a single bond or a methylene group that may have a substituent; X² represents a C₂₋₆ alkenyl group that may have a substituent, a C₂₋₆ alkynyl group that may have a substituent, or a phenyl group that may have a substituent);
   [10] a preventive or therapeutic agent for multiple sclerosis, which comprises the compound of any one of [1] to [8], or a salt or hydrate thereof, wherein X described above is a group represented by the formula —X¹¹—X¹² (where X¹¹ represents a single bond or a methylene group; X¹² represents a C₂₋₆ alkenyl group, a C₂₋₆ alkynyl group, or a phenyl group that may have a substituent);
   [11] a preventive or therapeutic agent for multiple sclerosis, which comprises the compound of [9] or [10], or a salt or hydrate thereof, wherein the phenyl group that may have at position 2 a substituent selected from the group consisting of:
   a hydroxyl group, a fluorine atom, a chlorine atom, a methyl group, an ethyl group, a fluoromethyl group, a vinyl group, a methoxy group, an ethoxy group, an acetyl group, a cyano group, a formyl group, and a C₂₋₇ alkoxycarbonyl group;
   [12] a preventive or therapeutic agent for multiple sclerosis, which comprises the compound of any one of [1] to [8], or a salt or hydrate thereof, wherein X is a 3-methyl-2-buten-1-yl group, a 2-butyne-1-yl group, a benzyl group, or a 2-chlorophenyl group;
   [13] a preventive or therapeutic agent for multiple sclerosis, which comprises the compound of any one of [1] to [8], or a salt or hydrate thereof, wherein X is a 2-butyne-1-yl group;
   [14] a preventive or therapeutic agent for multiple sclerosis, which comprises the compound of any one of [1] to [13], or a salt or hydrate thereof, wherein R¹ is a hydrogen atom or a group represented by the formula -A¹⁰-A¹¹-A¹²
   (wherein, A¹⁰ represents a C₁₋₆ alkylene group that may have one to three groups selected from substituent group C described below;
   A¹¹ represents a single bond, an oxygen atom, a sulfur atom, or a carbonyl group;
   A¹² represents a hydrogen atom, a C₆₋₁₀ aryl group that may have one to three groups selected from substituent group C described below, a 5- to 10-membered heteroaryl group that may have one to three groups selected from substituent group C described below, a 5- to 10-membered heteroaryl C₁₋₆ alkyl group that may have one to three groups selected from substituent group C described below, or a C₆₋₁₀ aryl C₁₋₆ alkyl group that may have one to three groups selected from substituent group C described below);
   <Substituent group C>
   substituent group C refers to a group consisting of:
   a hydroxyl group, a nitro group, a cyano group, a halogen atom, a C₁₋₆ alkyl group, a C₁₋₆ alkoxy group, a C₁₋₆ alkylthio group, a trifluoromethyl group, a group represented by the formula —NR^{C1}—R^{C2} (where R^{C1} and R^{C2} each independently represent a hydrogen atom or a C₁₋₆ alkyl group), and groups represented by the formulae —CO—R^{C3}—R^{C4} and —CH₂—CO—R^{C3}—R^{C4} (where R^{C3} represents a single bond, an oxygen atom, or a formula —NR^{C5}—; and R^{C4} and R^{C5} each independently represent a hydrogen atom or a C₁₋₆ alkyl group);
   [15] a preventive or therapeutic agent for multiple sclerosis, which comprises the compound of any one of [1] to [13], or a salt or hydrate thereof, wherein R¹ described above is a hydrogen atom, a C₁₋₆ alkyl group that may have one to three groups selected from substituent group C described below, a 5- to 10-membered heteroaryl C₁₋₆ alkyl group that may have one to three groups selected from substituent group C described below, or a C₆₋₁₀ aryl C₁₋₆ alkyl group that may have one to three groups selected from substituent group C described below;
   <Substituent group C>
   substituent group C refers to a group consisting of:
   a hydroxyl group, a nitro group, a cyano group, a halogen atom, a C₁₋₆ alkyl group, a C₁₋₆ alkoxy group, a C₁₋₆ alkylthio group, a trifluoromethyl group, a group represented by the formula —NR^{C1}—R^{C2} (where R^{C1} and R^{C2} each independently represent a hydrogen atom or a C₁₋₆ alkyl group), and groups represented by the formulae —CO—R^{C3}—R^{C4} and —CH₂—CO—R^{C3}—R^{C4} (where R^{C3} represents a single bond, an oxygen atom, or a formula —NR^{C5}—; and R^{C4} and R^{C5} each independently represent a hydrogen atom or a C₁₋₆ alkyl group);
   [16] a preventive or therapeutic agent for multiple sclerosis, which comprises the compound of [14] or [15], or a salt or hydrate thereof, wherein substituent group C consists of a cyano group, a C₁₋₆ alkoxy group, a C₂₋₇ alkoxycarbonyl group, and halogen atom;
   [17] a preventive or therapeutic agent for multiple sclerosis, which comprises the compound of any one of [1] to [13], or a salt or hydrate thereof, wherein R¹ described above is a methyl group, a cyanobenzyl group, fluorocyanobenzyl group, a phenethyl group, a 2-methoxyethyl group, or a 4-methoxycarbonylpyridin-2-yl group;
   [18] a preventive or therapeutic agent for multiple sclerosis, which comprises the compound of any one of [1] to [13], or a salt or hydrate thereof, wherein R¹ is a methyl group or a 2-cyanobenzyl group;
   [19] a preventive or therapeutic agent for multiple sclerosis, which comprises the compound of any one of [1] to [18], or a salt or hydrate thereof, wherein R² is a hydrogen atom, a cyano group, or a group represented by the formula -A²¹-A²² (where A²¹ represents a single bond, an oxygen atom, a sulfur atom, a sulfinyl group, a sulfonyl group, a carbonyl group, a formula —O—CO—, a formula —CO—O—, a formula —NR^{A2}—, a formula —CO—NR^{A2}—, or a formula —NR^{A2}—CO—; A²² and R^{A2} each independently represent a hydrogen atom, a cyano group, a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group; with the proviso that A²² and R^{A2} each independently may have one to three groups selected from substituent group D described below);
   <Substituent group D>
   substituent group D refers to a group consisting of:
   a hydroxyl group, a cyano group, a nitro group, a halogen atom, a C₁₋₆ alkyl group, a C₁₋₆ alkoxy group, a C₁₋₆ alkylthio group, a trifluoromethyl group, a group represented by the formula —NR^{D1}—R^{D2} (where R^{D1} and R^{D2} each independently represent a hydrogen atom or a C₁₋₆ alkyl group), a group represented by the formula —CO—R^{D3} (where R^{D3} represents a 4- to 8-membered heterocyclic group), and a group represented by the formula —CO—R^{D4}—R^{D5} (where R^{D4} represents a single bond, an oxygen atom, or a formula —NR^{D6}—; R^{D5} and R^{D6} each independently represent a hydrogen atom, a C₃₋₈ cycloalkyl group, or a C₁₋₆ alkyl group);
   [20] a preventive or therapeutic agent for multiple sclerosis, which comprises the compound of any one of [1] to [18], or a salt or hydrate thereof, wherein R² described above is a hydrogen atom, a cyano group, a carboxy group, a C₂₋₇ alkoxycarbonyl group, a C₁₋₆ alkyl group, a group represented by the formula —CONR^{D7}R^{D8} (wherein R^{D7} and R^{D8} each independently represent a hydrogen atom or a C₁₋₆ alkyl group), or a group represented by the formula -A²³-A²⁴ (where A²³ represents an oxygen atom, a sulfur atom, or a formula —NR^{A3}—; A²⁴ and R^{A3} each independently represent a hydrogen atom, a C₁₋₆ alkyl group that may have a group selected from substituent group D1 described below, a C₃₋₈ cycloalkyl group that may have a group selected from substituent group D1 described below, a C₂₋₆ alkenyl group that may have a group selected from substituent group D1 described below, a C₂₋₆ alkynyl group that may have a group selected from substituent group D1 described below, a phenyl group that may have a group selected from substituent group D1 described below, or a 5- to 10-membered heteroaryl group that may have a group selected from substituent group D1 described below);
   <Substituent group D1>
   substituent group D1 refers to a group consisting of:
   a carboxy group, a C₂₋₇ alkoxycarbonyl group, a C₁₋₆ alkyl group, a group represented by the formula —CONR^{D7}R^{D8} (wherein R^{D7} and R^{D8} each independently represent a hydrogen atom or a C₁₋₆ alkyl group), a pyrrolidin-1-ylcarbonyl group, a C₁₋₆ alkyl group, and a C₁₋₆ alkoxy group;

[21] a preventive or therapeutic agent for multiple sclerosis, which comprises the compound of any one of [1] to [18], or a salt or hydrate thereof, wherein R² described above is a hydrogen atom, a methyl group, a cyano group, a C₁₋₆ alkoxy group, or a group represented by the formula -A²⁵-A²⁶ (where A²⁵ represents an oxygen atom, a sulfur atom, or a formula —NR^{A4}—; A²⁶ and R^{A4} each independently represent a hydrogen atom, a C₁₋₆ alkyl group that may have a group selected from substituent group D1 described below, a C₃₋₈ cycloalkyl group that may have a group selected from substituent group D1 described below, or a phenyl group that may have a group selected from substituent group D1 described below);

<Substituent group D1>

substituent group D1 refers to a group consisting of:

a carboxy group, a C₂₋₇ alkoxycarbonyl group, a C₁₋₆ alkyl group, a group represented by the formula —CONR^{D7}R^{D8} (wherein R^{D7} and R^{D8} each independently represent a hydrogen atom or a C₁₋₆ alkyl group), a pyrrolidin-1-ylcarbonyl group, a C₁₋₆ alkyl group, and a C₁₋₆ alkoxy group;

In [21] above, when compound (I) is compound (II) or (III), more preferably, the above-defined R² represents a hydrogen atom, a cyano group, a C₁₋₆ alkoxy group, or a group represented by the formula -A²⁵-A²⁶ (where A²⁵ represents an oxygen atom, a sulfur atom, or a formula —NR^{A4}—; A²⁶ and R^{A4} each independently represent a hydrogen atom, a C₁₋₆ alkyl group having a substituent selected from substituent group D1 described above, a C₃₋₈ cycloalkyl group having a substituent selected from substituent group D1 described above, or a phenyl group having a substituent selected from substituent group D1 described above);
[22] a preventive or therapeutic agent for multiple sclerosis, which comprises the compound of any one of [1] to [18], or a salt or hydrate thereof, wherein R² described above is a hydrogen atom, a cyano group, a methoxy group, a carbamoylphenyloxy group, or a group represented by the following formula: 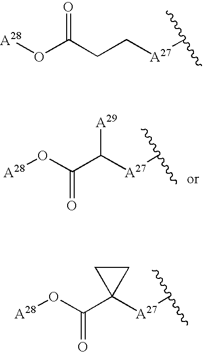
(where A²⁷ represents an oxygen atom, a sulfur atom, or —NH—; A2′ and A²⁹ each independently represent a hydrogen atom or a C₁₋₆ alkyl group);
[23] a preventive or therapeutic agent for multiple sclerosis, which comprises the compound of any one of [1] to [18], or a salt or hydrate thereof, wherein R² described above is a hydrogen atom, a cyano group, or a 2-carbamoylphenyloxy group;
[24] a preventive or therapeutic agent for multiple sclerosis, which comprises the compound of [1], or a salt or hydrate thereof, wherein the compound represented by formula (I) is any one of the compounds selected from the group consisting of:
7-(2-butynyl)-1,3-dimethyl-8-(piperazin-1-yl)-3,7-dihydropurine-2,6-dione,
7-(2-butynyl)-2-cyano-1-methyl-8-(piperazin-1-yl)-1,7-dihydropurin-6-one,
3-(2-butynyl)-5-methyl-2-(piperazin-1-yl)-3,5-dihydroimidazo[4,5-d]pyridazin-4-one,
2-(3-aminopiperidin-1-yl)-3-(2-butynyl)-5-methyl-3,5-dihydroimidazo[4,5-d]pyridazin-4-one,
2-[7-(2-butynyl)-1-methyl-6-oxo-8-(piperazin-1-yl)-6,7-dihydro-1H-purin-2-yloxy]benzamide,
7-(2-butynyl)-1-(2-cyanobenzyl)-6-oxo-8-(piperazin-1-yl)-6,7-dihydro-1H-purine-2-carbonitrile, and
2-[3-(2-butynyl)-4-oxo-2-(piperazin-1-yl)-3,4-dihydroimidazo[4,5-d]pyridazin-5-ylmethyl]benzo nitrile; and
   [25] a preventive or therapeutic agent for multiple sclerosis, which comprises the compound of [1], or a salt or hydrate thereof, wherein the compound represented by formula (I) is any one of the compounds selected from the group consisting of:
7-(2-butynyl)-2-cyano-1-methyl-8-(piperazin-1-yl)-1,7-dihydropurin-6-one,
3-(2-butynyl)-5-methyl-2-(piperazin-1-yl)-3,5-dihydroimidazo[4,5-d]pyridazin-4-one,
2-(3-aminopiperidin-1-yl)-3-(2-butynyl)-5-methyl-3,5-dihydroimidazo[4,5-d]pyridazin-4-one,
2-[7-(2-butynyl)-1-methyl-6-oxo-8-(piperazin-1-yl)-6,7-dihydro-1H-purin-2-yloxy]benzamide,
7-(2-butynyl)-1-(2-cyanobenzyl)-6-oxo-8-(piperazin-1-yl)-6,7-dihydro-1H-purine-2-carbonitrile, and
2-[3-(2-butynyl)-4-oxo-2-(piperazin-1-yl)-3,4-dihydroimidazo[4,5-d]pyridazin-5-ylmethyl]benzo nitrile.

The inventions of [5] to [8], in which the above T¹ is involved, the inventions of [9] to [13], in which the above X is involved, the inventions of [14] to [18] in which the above R¹ is involved, and the inventions of [19] to [23], in which the above R² is involved are preferred in this order in each series.

Among the compounds represented by formulae (II) to (IV) indicated in [2] to [4] above, compounds represented by the formula (II) or (III) are preferred. Furthermore, the inventions shown above in [5] to [23] are more preferably used when a compound represented by formula (II) or (III) is used.

The preventive or therapeutic agents for multiple sclerosis, which comprise the compound represented above by formula (I), more preferably by formula (II) or (III), include arbitrary combinations each selected from the groups of [5] to [8], [9] to [13], [14] to [18], and [19] to [23].

Herein below the terms and symbols used herein are defined and the present invention is described in detail.

Herein, a structural formula of a compound sometimes represents a certain isomer for convenience of description. However, compounds of the present invention may include all possible isomers, such as structurally possible geometric isomers, optical isomers generated due to the presence of asymmetric carbons, stereoisomers, tautomers, and mixtures of isomers, and are not limited to the formulae being used for convenience of description, and may be either of two isomers or a mixture. Thus, compounds of the present invention may be optically active compounds having an asymmetric carbon atom in their molecules or their racemates, but are not restricted in the present invention and can include any of these. Furthermore, compounds of the present invention may exhibit crystalline polymorphism, but likewise are not restricted to any one of these but may be in any one of these crystal forms or exist as a mixture of two or more crystal forms. Compounds of the present invention also include both anhydrous and hydrated forms. Compounds of the present invention also include solvates that have absorbed other types of solvents. Substances produced through in vivo metabolism of compounds of the invention are also within the scope of claims.

As used herein, the phrase “C₁₋₆ alkyl group” refers to a linear or branched alkyl group containing one to six carbon atoms, which is a monovalent group obtained by removal of any one hydrogen atom from an aliphatic hydrocarbon containing one to six carbons, and specifically, includes, for example, a methyl group, an ethyl group, a 1-propyl group, a 2-propyl group, a 2-methyl-1-propyl group, a 2-methyl-2-propyl group, a 1-butyl group, a 2-butyl group, a 1-pentyl group, a 2-pentyl group, a 3-pentyl group, a 2-methyl-1-butyl group, a 3-methyl-1-butyl group, a 2-methyl-2-butyl group, a 3-methyl-2-butyl group, a 2,2-dimethyl-1-propyl group, a 1-hexyl group, a 2-hexyl group, a 3-hexyl group, a 2-methyl-1-pentyl group, a 3-methyl-1-pentyl group, a 4-methyl-1-pentyl group, a 2-methyl-2-pentyl group, a 3-methyl-2-pentyl group, a 4-methyl-2-pentyl group, a 2-methyl-3-pentyl group, a 3-methyl-3-pentyl group, a 2,3-dimethyl-1-butyl group, a 3,3-dimethyl-1-butyl group, a 2,2-dimethyl-1-butyl group, a 2-ethyl-1-butyl group, a 3,3-dimethyl-2-butyl group, and a 2,3-dimethyl-2-butyl group.

As used herein, the phrase “C₂₋₆ alkenyl group” refers to a linear or branched alkenyl group containing two to six carbons, and specifically includes, for example, a vinyl group, an allyl group, a 1-propenyl group, a 2-propenyl group, a 1-butenyl group, a 2-butenyl group, a 3-butenyl group, a pentenyl group, and a hexenyl group.

As used herein, the phrase “C₂₋₆ alkynyl group” refers to a linear or branched alkynyl group containing two to six carbons, and specifically includes, for example, an ethynyl group, a 1-propynyl group, a 2-propynyl group, a butynyl group, a pentynyl group, and a hexynyl group.

As used herein, the phrase “C₃₋₈ cycloalkyl group” refers to a cyclic aliphatic hydrocarbon group containing three to eight carbon atoms, and specifically includes, for example, a cyclopropyl group, a cyclobutyl group, a cyclopentyl group, a cyclohexyl group, a cycloheptyl group, and a cyclooctyl group.

As used herein, a “C₃₋₇ cycloalkenyl group” refers to an unsaturated aliphatic hydrocarbon group containing three to seven carbon atoms, and specifically includes, for example, a cyclopropenyl group, a cyclobutenyl group, a cyclopentenyl group, a cyclohexenyl group, and a cycloheptenyl group, preferably a cyclopropenyl group, a cyclobutenyl group, a cyclopentenyl group, and a cyclohexenyl group.

As used herein, the phrase “C₁₋₆ alkylene group” refers to a divalent group obtained by removing another arbitrary hydrogen atom from the “C₁₋₆ alkyl group” defined above, and specifically includes, for example, a methylene group, a 1,2-ethylene group, a 1,1-ethylene group, a 1,3-propylene group, a tetramethylene group, a pentamethylene group, and a hexamethylene group.

As used herein, the phrase “C₃₋₈ cycloalkylene group” refers to a divalent group obtained by removing another arbitrary hydrogen atom from the “C₃₋₈ cycloalkyl group” defined above.

As used herein, the phrase “C₁₋₆ alkoxy group” refers to an oxy group linked to the “C₁₋₆ alkyl group” defined above, and specifically includes, for example, a methoxy group, an ethoxy group, a 1-propyloxy group, a 2-propyloxy group, a 2-methyl-1-propyloxy group, a 2-methyl-2-propyloxy group, a 1-butyloxy group, a 2-butyloxy group, a 1-pentyloxy group, a 2-pentyloxy group, a 3-pentyloxy group, a 2-methyl-1-butyloxy group, a 3-methyl-1-butyloxy group, a 2-methyl-2-butyloxy group, a 3-methyl-2-butyloxy group, a 2,2-dimethyl-1-propyloxy group, a 1-hexyloxy group, a 2-hexyloxy group, a 3-hexyloxy group, a 2-methyl-1-pentyloxy group, a 3-methyl-1-pentyloxy group, a 4-methyl-1-pentyloxy group, a 2-methyl-2-pentyloxy group, a 3-methyl-2-pentyloxy group, a 4-methyl-2-pentyloxy group, a 2-methyl-3-pentyloxy group, a 3-methyl-3-pentyloxy group, a 2,3-dimethyl-1-butyloxy group, a 3,3-dimethyl-1-butyloxy group, a 2,2-dimethyl-1-butyloxy group, a 2-ethyl-1-butyloxy group, a 3,3-dimethyl-2-butyloxy group, and a 2,3-dimethyl-2-butyloxy group.

As used herein, the phrase “C₁₋₆ alkylthio group” refers to a thio group linked to the “C₁₋₆ alkyl group” defined above, and specifically includes, for example, a methylthio group, an ethylthio group, a 1-propylthio group, a 2-propylthio group, a butylthio group, and a pentylthio group.

As used herein, the phrase “C₂₋₇ alkoxycarbonyl group” refers to a carbonyl group linked to the “C₁₋₆ alkoxy group” defined above, and specifically includes, for example, a methoxycarbonyl group, an ethoxycarbonyl group, a 1-propyloxycarbonyl group, and a 2-propyloxycarbonyl group.

As used herein, the phrase “C₂₋₇ alkylcarbonyl group” refers to a carbonyl group linked to the “C₁₋₆ alkyl group” defined above, and specifically includes, for example, a methylcarbonyl group, an ethylcarbonyl group, a 1-propylcarbonyl group, and a 2-propylcarbonyl group.

As used herein, the term “halogen atom” refers to a fluorine atom, a chlorine atom, a bromine atom, or an iodine atom.

As used herein, the phrase “C₆₋₁₀ aryl group” refers to an aromatic cyclic hydrocarbon group containing six to ten carbon atoms, and specifically includes, for example, a phenyl group, a 1-naphthyl group, and a 2-naphthyl group.

As used herein, an “alkylenedioxy group” refers to a divalent group represented by the formula —O—R—O— (where R is preferably an alkylene group having one to six carbon atoms, more preferably having one to four carbon atoms). The alkylenedioxy group includes, for example, a methylene dioxy, an ethylene dioxy, a trimethylene dioxy, a tetramethylene dioxy, —O—CH(CH₃)—O—, and —O—C(CH₃)₂—O—.

As used herein, the term “heteroatom” refers to a sulfur atom, an oxygen atom, or a nitrogen atom.

As used herein, the phrase “5- to 10-membered heteroaryl ring” refers to an aromatic 5 to 10-membered ring containing one or more heteroatoms, and specifically includes, for example, a pyridine ring, a thiophene ring, a furan ring, a pyrrole ring, an oxazole ring, an isoxazole ring, a thiazole ring, a thiadiazole ring, an isothiazole ring, an imidazole ring, a triazole ring, a pyrazole ring, a furazan ring, a thiadiazole ring, an oxadiazole ring, a pyridazine ring, a pyrimidine ring, a pyrazine ring, a triazine ring, indole ring, an isoindole ring, an indazole ring, a chromene ring, a quinoline ring, an isoquinoline ring, a cinnoline ring, a quinazoline ring, a quinoxaline ring, a naphthyridine ring, a phthalazine ring, a purine ring, a pteridine ring, a thienofuran ring, an imidazothiazole ring, a benzofuran ring, a benzothiophene ring, a benzoxazole ring, a benzothiazole ring, a benzothiadiazole ring, a benzimidazole ring, an imidazopyridine ring, a pyrrolopyridine ring, a pyrrolopyrimidine ring, and a pyridopyrimidine ring. Preferable “5 to 10-membered heteroaryl rings” include a pyridine ring, a thiophene ring, a furan ring, a pyrrole ring, an imidazole ring, a 1,2,4-triazole ring, a thiazole ring, a thiadiazole ring, a pyrazole ring, a furazan ring, a thiadiazole ring, a pyridazine ring, a pyrimidine ring, a pyrazine ring, an isoquinoline ring, a benzoxazole ring, a benzothiazole ring, and a benzimidazole ring. The most preferable example is a pyridine ring.

As used herein, the phrase “5- to I O-membered heteroaryl group” refers to a monovalent or divalent group obtained by removing any one or two hydrogen atoms from the “5- to 10-membered heteroaryl ring” described above.

As used herein, the phrase “4- to 8-membered heterocyclic ring” refers to a non-aromatic ring in which:

(i) the number of atoms constituting the ring is four to eight;

(ii) the atoms constituting the ring include one to two heteroatoms;

(iii) the ring may contain one to two double bonds;

(iv) the ring may contain one to three carbonyl groups; and

(v) the ring is monocyclic.

Specifically, the 4- to 8-membered heterocyclic ring includes, for example, an azetidine ring, a pyrrolidine ring, a piperidine ring, arazine ring, a thiazolidine ring, a dioxane ring, an imidazoline ring, a thiazoline ring, and a ring represented by one of the formulae: 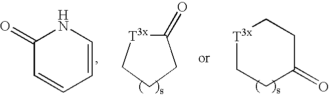
(where s represents an integer from 1 to 3; T^{3x} represents a methylene group, an oxygen atom or a group represented by the formula —NT^{4x}-, wherein T^{4x} represents a hydrogen atom or C₁₋₆ alkyl group. Preferably the “4- to 8-membered heterocyclic ring” includes a pyrrolidine ring, a piperidine ring, an azepan ring, a morpholine ring, a thiomorpholine ring, a piperazine ring, a dihydrofuran-2-one ring, and a thiazolidine ring.

As used herein, the phrase “4- to 8-membered heterocyclic group” refers to a monovalent or divalent group obtained by removing any one or two hydrogen atoms from a “4- to 8-membered heterocycle” described above. Preferably, the “4- to 8-membered heterocyclic groups” include a piperidin-1-yl group, a pyrrolidin-1-yl group, and a morpholin-4-yl group.

Herein, the cycloalkyl group or the 4- to 8-membered heterocyclic ring described above includes those condensed with an aryl group; and a “cycloalkyl group condensed with an aryl group” or a “4- to 8-membered heterocyclic ring condensed with an aryl group” refers to a structure comprising the cycloalkyl group or the 4- to 8-membered heterocyclic ring ortho-condensed with an aryl ring, such as a benzene ring. Specifically, the structure includes tetrahydronaphthalene, indane, and oxoindane, and preferably tetrahydronaphthalene and oxoindane.

As used herein, the phrase “C₆₋₁₀ aryl C₁₋₆ alkyl group” refers to a group obtained by substitution of a “C₆₋₁₀ aryl group” defined above for an arbitrary hydrogen atom in a “C₁₋₆ alkyl group” defined above, and specifically includes, for example, a benzyl group, a phenethyl group, and a 3-phenyl-1-propyl group.

As used herein, the phrase “5- to 10-membered heteroaryl C₁₋₆ alkyl group” refers to a group obtained by substitution of a “5- to I O-membered heteroaryl group” defined above for an arbitrary hydrogen atom in a “C₁₋₆ alkyl group” defined above, and specifically, includes, for example, a 2-pyridylmethyl and a 2-thienylmethyl group.

As used herein, the phrase “4- to 8-membered heterocyclic C₁₋₆ alkyl group” refers to a group obtained by substitution of a “4- to 8-membered heterocyclic group” defined above for an arbitrary hydrogen atom in a “C₁₋₆ alkyl group” defined above.

As used herein, the phrase “monocyclic or bicyclic 4- to 12-membered heterocyclic group containing one or two nitrogen atoms in the ring, which may have one or more substituents” refers to a non-aromatic cyclic group which may have one or more substituents. In the non-aromatic cyclic groups:

(i) the number of atoms constituting the ring of the cyclic group is four to 12;

(ii) the atoms constituting the ring of the cyclic group include one or two nitrogen atoms; and

(iii) the group is a monocyclic or bicyclic structure.

Specifically, the group is represented by the formula: 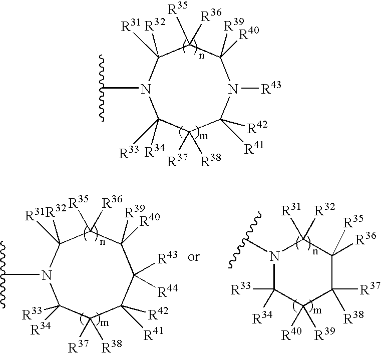
(where n and m each independently represent zero or one; R³¹ to R⁴⁴ independently represent a hydrogen atom or a substituent selected from the substituents referred to in the phrase “which may have one or more substituents” (the substituent group S defined below); any two of R³¹ to R⁴⁴ may in combination form a C₁₋₆ alkylene group).

Herein, the above-defined X may be linked with any one of R³¹, R³² and R³⁴, and in this case, X can be joined together with any one of R³¹, R³², R³³ and R³⁴ to form a ring structure.

As used herein, the phrase “which may have one or more substituents” means that a group may have one or more substituents in any combination at replaceable positions. Specifically, such substituents include, for example, a substituent selected from the substituent group S defined below:

<Substituent group S>

This group consists of:

(1) a halogen atom,

(2) a hydroxyl group,

(3) a mercapto group,

(4) a nitro group,

(5) a cyano group,

(6) a formyl group,

(7) a carboxy group,

(8) a trifluoromethyl group,

(9) a trifluoromethoxy group,

(10) an amino group,

(11) an oxo group,

(12) an imino group, and

(13) a group represented by the formula -T^{1x}-T^{2x}-T^{3x} (wherein,

T^{1x} is a single bond or a C₁₋₆ alkylene group,

T^{2x} is a single bond, a C₁₋₆ alkylene group, an oxygen atom, a group represented by the formula —CO—, a group represented by the formula —S—, a group represented by the formula —S(O)—, a group represented by the formula —S(O)₂—, a group represented by the formula —O—CO—, a group represented by the formula —CO—O—, a group represented by the formula —NR^{T}—, a group represented by the formula —CO—NR_{T}—, a group represented by the formula —NR_{T}—CO—, a group represented by the formula —SO₂—NR^{T}—, a group represented by the formula —NR^{T}—SO₂—, a group represented by the formula —NH—CO—NR^{T}— or a group represented by the formula —NH—CS—NR^{T}—; T^{3x} represents a hydrogen atom, a C₁₋₆ alkyl group, a C₃₋₈ cycloalkyl group, a C₂₋₆ alkenyl group, a C₂₋₆ alkynyl group, a phenyl group, a 1-naphthyl group, a 2-naphthyl group, a 5- to 10-membered heteroaryl group or a 4- to 8-membered heterocyclic group;

R^{T} represents a hydrogen atom, a C₁₋₆ alkyl group, a C₃₋₈ cycloalkyl group, a C₂₋₆ alkenyl group or a C₂₋₆ alkynyl group;

provided that T^{3x} and R^{T} each may independently have one to three substituents selected from the substituent group T defined below).

<Substituent group T>

This group consists of: hydroxyl, cyano, a halogen atom, C₁₋₆ alkyl, C₃₋₈ cycloalkyl, C₂₋₆ alkenyl, C₂₋₆ alkynyl, phenyl, 1-naphthyl, 2-naphthyl, 5 to 10-membered heteroaryl, 4 to 8-membered heterocyclic ring, C₁₋₆ alkoxy, C₁₋₆ alkylthio, C₂₋₇ alkoxycarbonyl group, etc.

The <substituent group S> preferably consists of:

(1) a halogen atom,

(2) a hydroxyl group,

(3) a cyano group,

(4) a carboxy group,

(5) a trifluoromethyl group,

(6) a trifluoromethoxy group,

(7) an amino group,

(8) a C₁₋₆ alkyl group,

(9) a C₃₋₈ cycloalkyl group,

(10) a C₂₋₆ alkenyl group,

(11) a C₂₋₆ alkynyl group,

(12) a phenyl group, and

(13) a C₁₋₆ alkoxy group.

As used herein, the term “group represented by the formula: 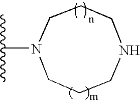
(where n and m each independently represent zero or one), which may have one or more substituents” refers to a group represented by the formula: 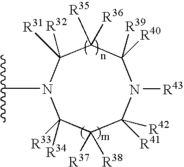
(where R³¹ to R⁴⁴ independently represent a hydrogen atom or a group selected from substituents referred to in the phrase “which may have one or more substituents” defined above (the substituent group S defined above); n and m each independently represent zero or one). The case where m=n=0 is preferred. More preferably, the term refers to a group represented by one of the formulae: 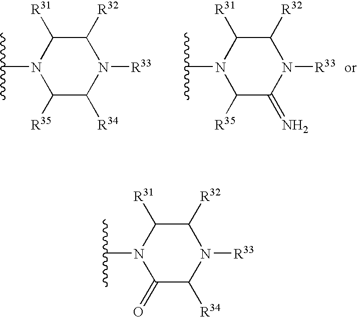
(where R³¹, R³², R³³, R³⁴, and R³⁵ independently represent a hydrogen atom or a group selected from substituent groups referred to in the phrase “which may have one or more substituents” (the substituent group S defined above)); provided that, at least three of R³¹, R³², R³³, R³⁴, and R³⁵ are hydrogen atoms Still more preferably, the term refers to a group represented by one of the formulae: 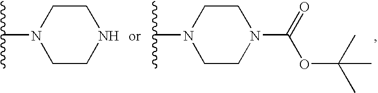
Most preferably, the term refers to a group represented by the formula:

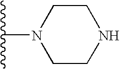

As used herein, the term “group represented by the formula: 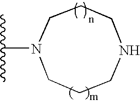
(where n and m each independently represent zero or one)” refers to a group represented by one of the formulae:

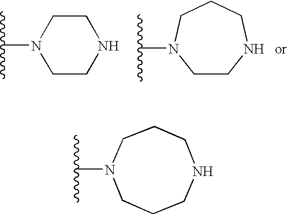

As used herein, the term “piperidin-1-yl group which may have one or more substituents” refers to a “piperidin-1-yl group” which may have one or more substituents selected from the groups referred to in the phrase “which may have one or more substituents” (the substituent group S defined above) at replaceable positions. Preferably, the “piperidin-1-yl group which may have one or more substituents” refers to a group represented by the formula: 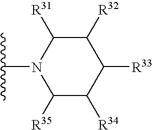
(where R³¹, R³², R³³, R³⁴, and R³⁵ each independently represent a hydrogen atom or a group selected from the substituents referred to in the phrase “which may have one or more substituents” (the substituent group S defined above)); provided that, at least three of R³¹, R³², R³³, R³⁴, and R³⁵ are hydrogen atoms. Preferably, the term refers to a group represented by one of the formulae: 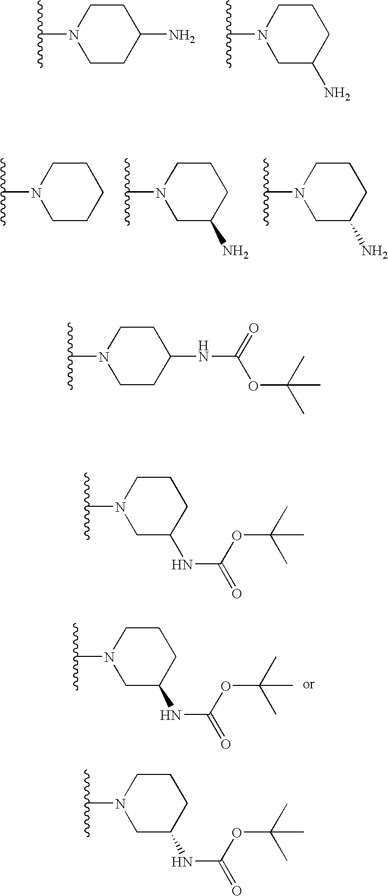
   More preferably, the term refers to a group represented by one of the formulae:

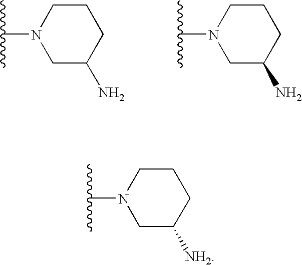

As used herein, the phrase “azetidin-1-yl group may have one or more substituents” refers to an “azetidin-1-yl group” which may have one or more groups selected from the substituents referred to in the phrase “which may have one or more substituents” at replaceable positions.

As used herein, the phrase “pyrrolidin-1-yl group may have one or more substituents” refers to a “pyrrolidin-1-yl group” which may have one or more groups selected from the substituents referred to in the phrase “which may have one or more substituents” at replaceable positions.

As used herein, the phrase “piperidin-1-yl group may have one or more substituents” refers to a “piperidin-1-yl group” which may have one or more groups selected from the substituents referred to in the phrase “which may have one or more substituents” at replaceable positions.

As used herein, the phrase “azepan-1-yl group may have one or more substituents” refers to an “azepan-1-yl group” which may have one or more groups selected from the substituents referred to in the phrase “which may have one or more substituents” at replaceable positions.

As used herein, the phrase “piperidin-1-yl group which may have an amino group” refers to a “piperidin-1-yl group” which may have an amino group at a replaceable position. Specifically, the “piperidin-1-yl group which may have an amino group”, for example, refers to a group represented by one of the formulae: 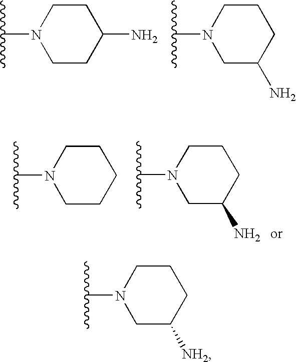
and preferably, to a group represented by one of the formulae:

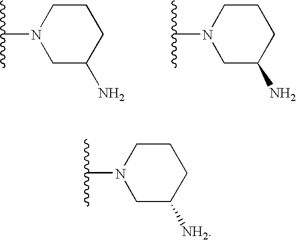

As used herein, the phrase “azetidin-1-yl group which may have an amino group” refers to an “azetidin-1-yl group” which may have an amino group at a replaceable position.

As used herein, the phrase “pyrrolidin-1-yl group which may have an amino group” refers to a “pyrrolidin-1-yl group” which may have an amino group at a replaceable position.

As used herein, the phrase “piperidin-1-yl group which may have an amino group” refers to a “piperidin-1-yl group” which may have an amino group at a replaceable position.

As used herein, the phrase “azepan-1-yl group which may have an amino group” refers to an “azepan-1-yl group” which may have an amino group at a replaceable position.

As used herein, the phrase “C₁₋₆ alkyl group which may have one or more substituents” in the substituent group B defined above refers to a “C₁₋₆ alkyl group” which may have one or more groups selected from the substituents referred to in the phrase “which may have one or more substituents” at replaceable positions. Preferably, the “C₁₋₆ alkyl group which may have one or more substituents” refers to a C₁₋₆ alkyl group which may have one or two substituents selected from the group consisting of a cyano group, a carboxy group, a C₂₋₇ alkoxycarbonyl group, a group represented by the formula —NR^{3T}COR^{4T}, a group represented by the formula —CONR^{3T}R^{4T} (where R^{3T} and R^{4T} each independently represent a hydrogen atom or a C₁₋₆ alkyl group), and a C₁₋₆ alkoxy group.

In a compound represented by formula (I) indicated above, R¹ and R² each independently represent a group of the formula -A⁰-A¹-A² (where A⁰, A¹ and A² are as defined above); when both A⁰ and A¹ are single bonds, “-A⁰-A¹-” represents a single bond.

In formula (I) indicated above, the phrase “when Z² represents a group of the formula —CR²═, R¹, and R² may in combination form a 5- to 7-membered ring” means that compounds represented by formula (I) indicated above include compounds (V) represented by the formula: 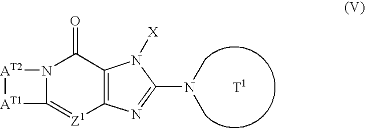
(where Z¹, X, and T¹ are as defined above; A^{T1} represents an oxygen atom, a sulfur atom, a sulfinyl group, a sulfonyl group, a carbonyl group, a methylene group which may have one or more substituents, or a nitrogen atom which may have one or more substituents; A^{T2} represents a C₂₋₆ alkylene group which may have one or more substituents). In formula (V) shown above, A^{T1} preferably represents an oxygen atom, and A^{T2} preferably represents a C₂₋₄ alkylene group.

As used herein, the phrase “cyanobenzyl group” refers to a benzyl group having one cyano group, and specifically includes, for example, a 2-cyanobenzyl group, a 3-cyanobenzyl group, and a 4-cyanobenzyl group.

As used herein, the phrase “fluorocyanobenzyl group” refers to a benzyl group having one fluorine atom and one cyano group, and specifically includes, for example, a 2-cyano-4-fluorobenzyl group and a 2-cyano-6-fluorobenzyl group.

As used herein, the phrase “carbamoylphenoxy group” refers to a phenoxy group having a group represented by the formula —CONH₂, and specifically includes, for example, a 2-carbamoylphenoxy group, a 3-carbamoylphenoxy group, and a 4-carbamoylphenoxy group.

Herein, there is no limitation on the type of a “salt” as long as the salts are pharmaceutically acceptable and derived from any compound of the present invention. Such salts include, for example, inorganic acid salts, organic acid salts, inorganic base salts, organic base salts, and acidic or basic amino acid salts.

Examples of preferred inorganic salts include hydrochloride, hydrobromide, sulfate, nitrate, and phosphate. Examples of preferred organic salts include acetate, succinate, fumarate, maleate, tartrate, citrate, lactate, stearate, benzoate, methanesulfonate, and p-toluene sulfonate.

Examples of preferred inorganic base salts include: alkali metal salts such as sodium salts and potassium salts; alkaline earth metal salts such as calcium salts and magnesium salts; aluminum salts; and ammonium salts. Examples of preferred organic base salts include diethylamine salts, diethanolamine salts, meglumine salts, and N,N′-dibenzylethylenediamine salts.

Examples of preferred acidic amino acid salts include aspartate and glutamate. Examples of preferred basic amino acid salts include arginine salts, lysine salts, and ornithine salts.

[Typical Synthesis Methods]

Representative methods for producing compounds of the present invention, represented by formula (I) above, are described below.

Each symbol in the production methods is defined below. R³¹ to R⁴², n, m, R¹, R², X, A⁰, A¹, A², R^{A}, and T¹ are the same as defined above.

U¹ and U³ each independently represent a leaving group, such as a chlorine atom, a bromine atom, an iodine atom, a methanesulfonyloxy group, or a p-toluenesulfonyloxy group.

R^{p1}, R^{p2}, and R^{p3} each independently represent an —NH-protecting group, such as a pivalyloxymethyl group and a trimethylsilylethoxymethyl group.

R^{p4} represents a hydroxyl group-protecting group, such as a t-butyldimethylsilyl group and a t-butyldiphenylsilyl group.

R^{p5} represents an NH-protecting group, such as N,N-dimethylsulfamoyl, trityl, benzyl, and t-butoxycarbonyl.

U² and U⁴ each independently represent a chlorine atom, a bromine atom, an iodine atom, a methanesulfonyloxy group, a p-toluenesulfonyloxy group, a group represented by the formula —B(OH)₂, a 4,4,5,5-tetramethyl-1,3,2-dioxaboran-2-yl group, or a group represented by the formula —Sn(R^{z})₃ (where R^{z} represents a C₁₋₆ alkyl group).

R^{x2} is a group represented by the formula —O-A², a group represented by the formula —S-A², a group represented by the formula —N(R^{A})A², or a 4- to 8-membered heterocyclic group which may have one or more substituents (for example, 1-pyrrolidinyl, 1-morpholinyl, 1-piperazinyl, or 1-piperidyl), etc.

R^{x3} represents a group of the formula -A⁰-A¹-A², such as a cyano group, a C₁₋₆ alkyl group which may have one or more substituents, a C₃₋₈ cycloalkyl group which may have one or more substituents, a C₂₋₆ alkenyl group which may have one or more substituents, a C₂₋₆ alkynyl group which may have one or more substituents, and a C₆₋₁₀ aryl group which may have one or more substituents.

A^{2COOR} represents a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group, each of which contains an ester group.

A^{2COOH} represents a C₁₋₆ alkyl group, a C₃₋₈ cycloalkyl group, a C₂₋₆ alkenyl group, a C₂₋₆ alkynyl group, C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group, each of which contains a carboxylic acid.

A^{2NO2} represents a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group, each of which contains a nitro group.

A^{2NH2} represents a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group, each of which contains an amino group.

A^{2CN} represents a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group, each of which contains a nitrile group.

A^{CONH2} represents a C₁₋₆ alkyl group, a C₃₋₈ cycloalkyl group, a C₂₋₆ alkenyl group, a C₂₋₆ alkynyl group, C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group, each of which contains a carboxylic amide group.

M represents —MgCl, —MgBr, —Sn(R^{Z})₃ (where R^{z} is as defined above), etc.

The term “room temperature” refers to a temperature of about 20° C. to about 30° C.

T^{1a} is defined as the group represented by T¹, or represents a group of the formula: 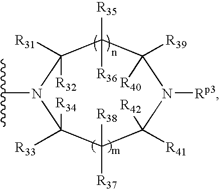
a group represented by the formula: 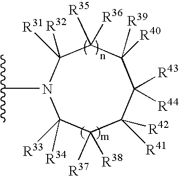
(where R³¹ to R⁴⁴ are as defined above, except that any one of R³¹ to R⁴⁴ represents —NH—R³), or a group represented by the formula: 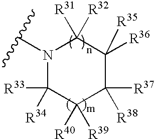
(where R³¹ to R⁴⁰ are as defined above, except that any one of R³¹ to R⁴⁰ represents —NH—R³).

In examples of reactions represented by the following reaction schemes, unless otherwise specified, the quantities of reagents, catalysts, and others to be used (equivalent, weight %, and weight ratio) are represented as ratios to a main compound in each reaction scheme. The main compound refers to a compound represented by a chemical formula in the reaction scheme and having a backbone of compounds of the present invention.

Method A to Q for producing compounds in which Z¹ and Z² are conjugated with a double bond, represented by formula (I) above, are described below.
Production Method A 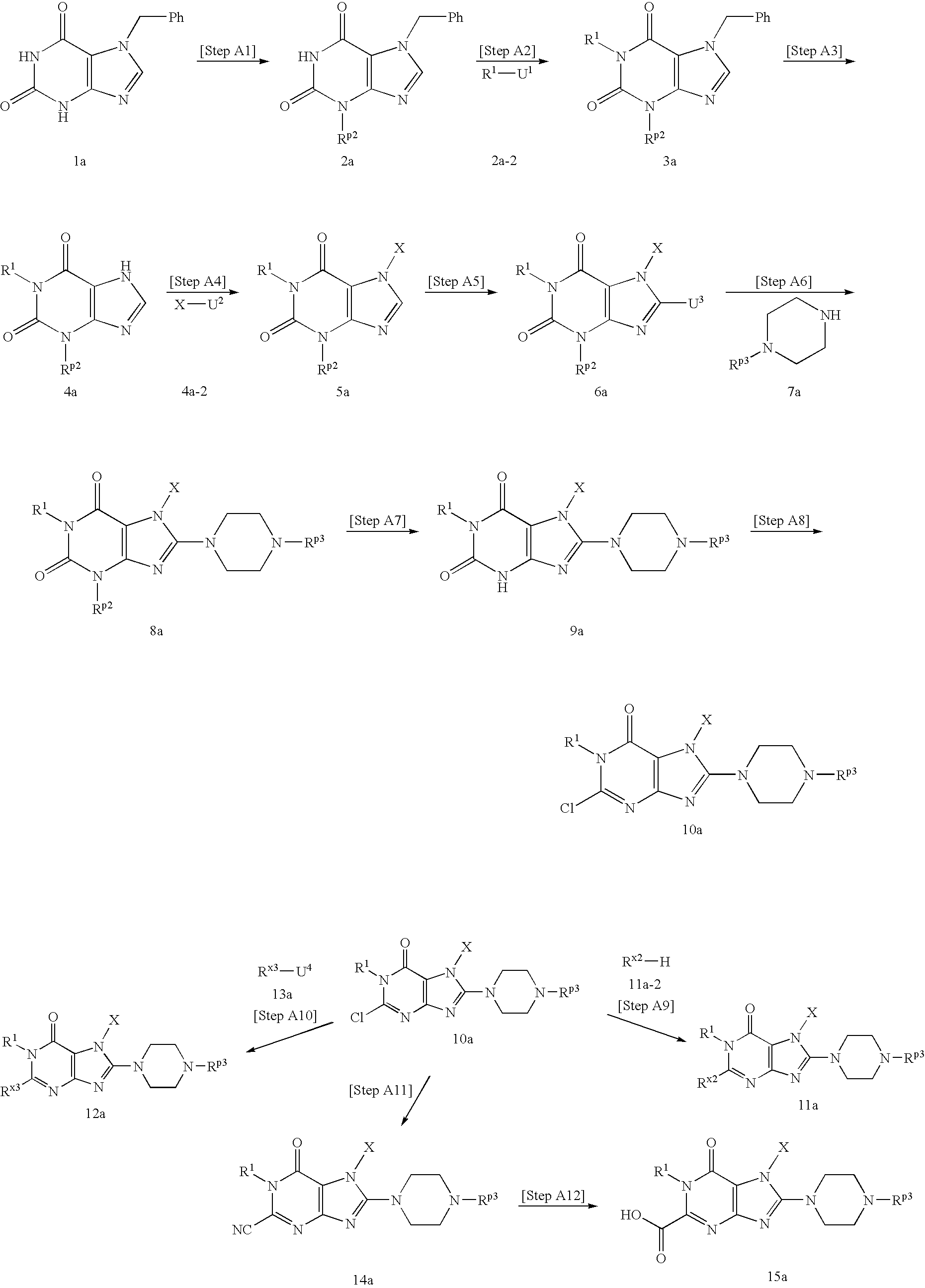 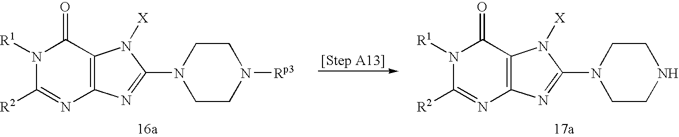
[Step A1]

In this step, an —NH-protecting reagent is reacted with compound (1a) [CAS No. 56160-64-6] to give compound (2a). The reaction conditions are selected depending on the type of —NH-protecting reagent to be used. The reaction may be performed under conditions that are generally used to introduce a protecting group using the reagent.

An —NH-protecting reagent can be a reagent that is generally used to introduce an —NH-protecting group. Specifically, such —NH-protecting reagents include, for example, chloromethyl pivalate. It is preferable to use one to two equivalents of a protecting reagent. Solvents for the reaction include acetonitrile, N,N-dimethylformamide, N-methylpyrrolidone, 1,4-dioxane, tetrahydrofuran, and dimethoxyethane. N,N-dimethylformamide is preferably used.

The reaction can be achieved in the presence of a base. Examples of bases to be used in the reaction include cesium carbonate, lithium carbonate, sodium carbonate, potassium carbonate, and sodium hydride. Sodium hydride is preferably used. In this case, a base is preferably used in an amount of one to five equivalents. The reaction can be conducted at a temperature ranging from 0° C. to 150° C. A preferred reaction temperature is room temperature.

[Step A2]

In this step, compound (2a) is reacted with compound (2a-2) to give compound (3a).

Compound (2a-2) can be any compound that is an electrophilic reagent such as an alkyl halide. Specific examples include alkyl halides such as iodomethane, iodoethane, iodopropane, and benzyl bromide; alkenyl halides such as allyl bromide and 1-bromo-3-methyl-2-butene; and alkynyl halides such as propargyl bromide and 1-bromo-2-butyne. One to two equivalents of an electrophilic reagent are preferably used.

Solvents for the reaction include, for example, dimethyl sulfoxide, N,N-dimethylformamide, N-methylpyrrolidone, dioxane, tetrahydrofuran, and toluene.

The reaction can be achieved in the presence or absence of a base. Examples of bases to be used in the reaction include lithium hydroxide, sodium hydroxide, potassium hydroxide, lithium carbonate, sodium carbonate, potassium carbonate, cesium carbonate, lithium hydride, sodium hydride, potassium hydride, butyllithium, methyllithium, lithium bis(trimethylsilyl)amide, sodium bis(trimethylsilyl)amide, and potassium bis(trimethylsilyl)amide. In this case, one to two equivalents of a base are preferably used. The reaction can be conducted at a temperature ranging from 0° C. to 150° C.

[Step A3]

In this step, the benzyl group at the 7-position is removed from compound (3a) to give compound (4a).

Specifically, compound (4a) can be prepared from compound (3a), for example, by catalytic reduction under a hydrogen atmosphere in the presence of a metal catalyst, but the reaction conditions are not limited thereto.

Specific solvents for the reaction include, for example, methanol, ethanol, propanol, acetic acid, dimethyl sulfoxide, N,N-dimethylformamide, N-methylpyrrolidone, dioxane, tetrahydrofuran, and toluene. Examples of metal catalysts include palladium carbon, platinum oxide, and Raney nickel. A metal catalyst is preferably used at 0.5 to 50 weight %. A preferred hydrogen pressure is 1 to 5 atm. The reaction can be conducted at a temperature ranging from 0° C. to 150° C.

[Step A4]

In this step, compound (4a) is reacted with compound (4a-2) to give compound (5a).

Specific examples of compound (4a-2) are: alkyl halides such as iodomethane, iodoethane, iodopropane, and benzyl bromide; alkenyl halides such as allyl bromide and 1-bromo-3-methyl-2-butene; or alkynyl halides such as propargyl bromide and 1-bromo-2-butyne. These halides are preferably used in an amount of one to two equivalents.

Solvents for the reaction include dimethyl sulfoxide, N,N-dimethylformamide, N-methylpyrrolidone, dioxane, tetrahydrofuran, and toluene.

The reaction can be carried out in the presence or absence of a base. Examples of bases to be used in the reaction include lithium hydroxide, sodium hydroxide, potassium hydroxide, lithium carbonate, sodium carbonate, potassium carbonate, cesium carbonate, lithium hydride, sodium hydride, potassium hydride, butyllithium, methyllithium, lithium bis(trimethylsilyl)amide, sodium bis(trimethylsilyl)amide, and potassium bis(trimethylsilyl)amide. In this case, one to four equivalents of a base are preferably used. The reaction can be conducted at a temperature ranging from 0° C. to 150° C.

Compound (5a) can be obtained by reacting compound (4a) with compound (4a-2) in the presence of a copper catalyst and a base. In this case, it is preferable to use 0.1 to two equivalents of a copper catalyst and one to ten equivalents of a base.

In this reaction, compound (4a-2) may be arylboronic acid, heteroarylboronic acid, or such, in which X is a C₆₋₁₀ aryl group which may have one or more substituents or a 5- to 10-membered heteroaryl group which may have one or more substituents, and U² is —B(OH)₂ or such. One to three equivalents of compound (4a-2) are preferably used.

In this case, reaction solvents include dichloromethane, chloroform, 1,4-dioxane, tetrahydrofuran, toluene, pyridine, N,N-dimethylformamide, and N-methylpyrrolidone.

Bases include triethylamine, diisopropyl ethyl amine, pyridine, and N,N-dimethylaminopyridine. Copper catalysts include copper (II) acetate, copper (II) trifluoroacetate, copper (II) chloride, and copper (II) iodide. The reaction can be conducted at a temperature ranging from 0° C. to 150° C.

[Step A5]

In this step, compound (5a) is reacted with a halogenating agent to give compound (6a).

Specific examples of halogenating agents include, for example, N-chlorosuccinimide, N-bromosuccinimide, and N-iodosuccinimide. A halogenating agent is preferably used in an amount of one to four equivalents.

Solvents for the reaction include acetonitrile, N,N-dimethylformamide, N-methylpyrrolidone, 1,4-dioxane, tetrahydrofuran, and dimethoxyethane. The reaction can be conducted at a temperature ranging from 0° C. to 150° C.

[Step A6]

In this step, compound (6a) is reacted with compound (7a) to give compound (8a). In this case, one to four equivalents of compound (7a) are preferably used.

The reaction can be carried out, for example, in a solvent such as tetrahydrofuran, acetonitrile, N,N-dimethylformamide, N-methylpyrrolidone, methanol, ethanol, 1,4-dioxane, toluene, or xylene, or in the absence of a solvent. The reaction can be conducted at a temperature ranging from 0° C. to 200° C. in the presence or absence of a base. Examples of a base include triethylamine, potassium carbonate, and 1,8-diazabicyclo[5,4,0]undecene. In this case, one to four equivalents of base are preferably used.

[Step A7]

In this step, the —NH-protecting group at the 3-position of compound (8a) is removed to give compound (9a). The reaction conditions are selected depending on the type of —NH-protecting group to be removed. The deprotection reaction may be performed under conditions that are generally used for the protecting group.

For example, when R^{p2} is a pivalyloxymethyl group, the reaction can be carried out in methanol, or a mixed solution of methanol and tetrahydrofuran, using a base such as sodium methoxide, sodium hydride, or 1,8-diazabicyclo[5,4,0]-7-undecene at a temperature of 0° C. to 150° C. In this case, 0.1 to two equivalents of base are preferably used.

Alternatively, when R^{p2} is a trimethylsilylethoxymethyl group, the reaction can be carried out in a solvent such as acetonitrile, N,N-dimethylformamide, N-methylpyrrolidone, 1,4-dioxane, tetrahydrofuran, or dimethoxyethane, using a fluoride reagent such as tetrabutyl ammonium fluoride or cesium fluoride at a temperature of 0° C. to 150° C. In this case, one to five equivalents of a fluoride reagent are preferably used.

[Step A8]

In this step, compound (9a) is chlorinated to give compound (10a).

There are no particular limitations on the reaction conditions, and the reaction can be conducted under standard conditions for chlorination. For example, the reaction can be carried out at a temperature ranging from 0° C. to 150° C. in a solvent such as phosphorus oxychloride. In this case, it is preferable to use 10 to 200 times the amount of halogenating agent by weight.

When R^{p3} is a t-butoxycarbonyl group or such, which is removed under the above-described conditions using phosphorus oxychloride or such, the protecting group should be reintroduced.

There are no particular limitations on the reaction conditions for protection. In the case of the t-butoxycarbonyl group, the reaction can be carried out using an —NH— protection reagent such as di-t-butyl dicarbonate, in a solvent such as acetonitrile, N,N-dimethylformamide, N-methylpyrrolidone, 1,4-dioxane, tetrahydrofuran, or dimethoxyethane in the presence of a base such as lithium hydroxide, sodium hydroxide, potassium hydroxide, lithium carbonate, sodium carbonate, potassium carbonate, cesium carbonate, potassium bicarbonate, sodium bicarbonate, or triethylamine at 0° C. to 150° C.

[Step A9]

In this step, compound (10a) is reacted with compound (11a-2) to give compound (11a).

Compound (11a-2) includes alcohol compounds or phenol compounds represented by A²-OH, amine compounds represented by A²(R^{A})NH or such, and thiol compounds represented by A²-SH. In this case, compound (11a-2) is preferably used in an amount of one to ten equivalents or five to 100 times by weight.

Solvents for the reaction include acetonitrile, N,N-dimethylformamide, N-methylpyrrolidone, 1,4-dioxane, tetrahydrofuran, dimethoxyethane, methanol, and ethanol.

The reaction can be carried out in the presence or absence of a base. Bases to be used in the reaction include lithium hydroxide, sodium hydroxide, potassium hydroxide, lithium carbonate, sodium carbonate, potassium carbonate, cesium carbonate, lithium hydride, sodium hydride, potassium hydride, butyllithium, methyllithium, lithium bis(trimethylsilyl)amide, sodium bis(trimethylsilyl)amide, potassium bis(trimethylsilyl)amide, and triethylamine. In this case, one to ten equivalents of base are preferably used. The reaction can be conducted at a temperature ranging from 0° C. to 150° C.

[Step A10]

In this step, compound (10a) is reacted with compound (13a) in the presence of a metal catalyst to give compound (12a). In this case, one to 50 equivalents of compound (13a) are preferably used.

Solvents for the reaction include acetonitrile, N,N-dimethylformamide, N-methylpyrrolidone, 1,4-dioxane, tetrahydrofuran, dimethoxyethane, methanol, and ethanol.

Metal catalysts include palladium catalyst and copper catalyst. Palladium catalysts include tetrakis triphenylphosphine palladium, palladium acetate, and dibenzylideneacetone palladium. Copper catalysts include copper iodide. It is preferable to use 0.01 to two equivalents of metal catalyst.

The reaction can be conducted in the presence of an organophosphorous ligand. When the reaction is carried out in the presence of an organophosphorous ligand, examples of the ligands include o-tolyl phosphine and diphenylphosphinoferrocene. In this case, it is preferable to use one to five equivalents of an organophosphorous ligand to the metal catalyst.

The reaction can be carried out in the presence or absence of a base. Bases to be used in the reaction include lithium hydroxide, sodium hydroxide, potassium hydroxide, lithium carbonate, sodium carbonate, potassium carbonate, cesium carbonate, lithium hydride, sodium hydride, potassium hydride, potassium phosphate, lithium bis trimethylsilyl amide, sodium bis trimethylsilyl amide, potassium bis trimethylsilyl amide, and triethylamine. The reaction can be conducted at a temperature ranging from 0° C. to 150° C.

[Step A11]

In this step, compound (10a) is reacted with a cyanidation reagent to give compound (14a).

Specifically, cyanidation reagents include, for example, sodium cyamide and potassium cyamide. They are preferably used in an amount of one to 20 equivalents.

Solvents for the reaction include, for example, acetonitrile, N,N-dimethylformamide, N-methylpyrrolidone, 1,4-dioxane, tetrahydrofuran, dimethoxyethane, methanol, and ethanol. The reaction can be conducted at a temperature ranging from 0° C. to 150° C.

[Step A12]

In this step, the cyano group of compound (14a) is hydrolyzed to give compound (15a). There are no particular limitations on the reaction conditions, and the reaction can be carried out under conditions generally used for the conversion of a cyano group to a carbamoyl group by hydrolysis.

Solvents for the reaction include N,N-dimethylformamide, N-methylpyrrolidone, 1,4-dioxane, tetrahydrofuran, dimethoxyethane, methanol, ethanol, and a mixed solvent of tetrahydrofuran and methanol.

The reaction can be carried out in the presence or absence of a base. When a base is used, the reaction can be carried out using an aqueous solution of a base such as potassium hydroxide, sodium hydroxide, lithium hydroxide, or ammonia. The reaction can be achieved after adding an aqueous solution of hydrogen peroxide (preferably an aqueous solution of 30% hydrogen peroxide).

The reaction can be conducted at a temperature ranging from 0° C. to 150° C.

[Step A13]

In this step, an R^{p3} of compound (16a) is removed to give compound (17a). Compounds (11a), (12a), (14a), (15a), and others can be used as compound (16a).

The deprotection reaction for R^{p3} can be carried out under standard reaction conditions for removing an —NH-protecting group.

For example, when R^{p3} is a t-butoxycarbonyl group, the reaction can be carried out in the presence of an acid, such as an anhydrous methanol solution of hydrogen chloride, an anhydrous ethanol solution of hydrogen chloride, an anhydrous dioxane solution of hydrogen chloride, trifluoroacetic acid, or formic acid.

An alternative method for producing compound (10a) is described below: 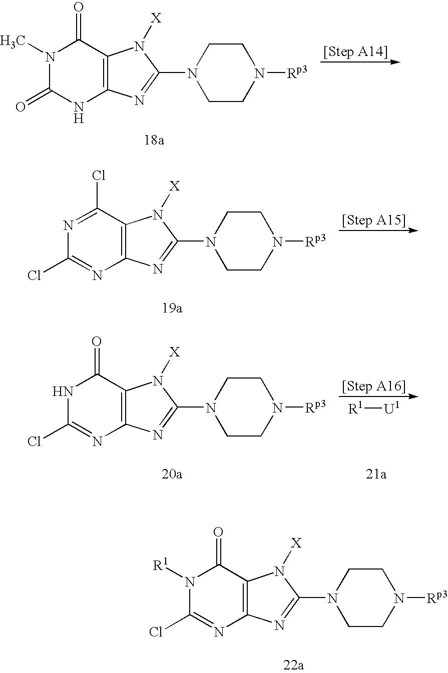
[Step A14]

In this step, compound (18a) is chlorinated to give compound (19a). There are no particular limitations on the reaction conditions, and the reaction can be conducted under standard conditions for chlorination. For example, the reaction can be carried out in a solvent such as phosphorus oxychloride at a temperature ranging from 0° C. to 150° C. Preferably ten to 200 times by weight of a chlorinating reagent is used.

When R^{p3} is a t-butoxycarbonyl group or such, which is removed under the above-described conditions using phosphorus oxychloride or such, the protecting group should be reintroduced.

There are no particular limitations on the reaction conditions for protection, and when R^{p3} is a t-butoxycarbonyl group, the reaction can be carried out using an —NH— protection reagent such as di-t-butyl dicarbonate, in a solvent such as acetonitrile, N,N-dimethylformamide, N-methylpyrrolidone, 1,4-dioxane, tetrahydrofuran, or dimethoxyethane, in the presence of a base such as lithium hydroxide, sodium hydroxide, potassium hydroxide, lithium carbonate, sodium carbonate, potassium carbonate, cesium carbonate, potassium bicarbonate, sodium bicarbonate, or triethylamine at a temperature ranging from 0° C. to 150° C.

[Step A15]

In this step, compound (19a) is partially hydrolyzed to give compound (20a). The reaction is carried out in the presence of a base such as sodium acetate, potassium carbonate, or sodium hydroxide. One to ten equivalents of base are preferably used. Solvents for the reaction include dimethyl sulfoxide, N-methylpyrrolidone, tetrahydrofuran, water, and mixtures thereof. The reaction can be conducted at a temperature ranging from 0° C. to 100° C.

[Step A16]

In this step, compound (20a) is reacted with compound (21a) to give compound (22a). The reaction can be conducted under the same conditions as used in [Step A2] of production method A.

An alternative method for producing compound (19a) is described below: 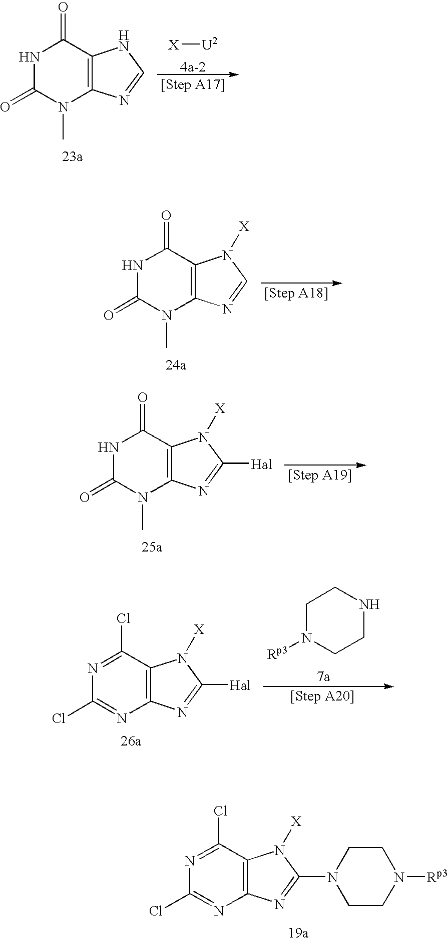
[Step A17]

In this step, a substitution reaction is carried out using compound (23a) [CAS No. 1076-22-8] and compound (4a-2) to give compound (24a).

The reaction can be conducted under the same conditions as used in [Step A4] of production method A.

[Step A18]

In this step, compound (24a) is reacted with a halogenating agent to give compound (25a).

The reaction can be conducted under the same conditions as used in [Step A5] of production method A.

[Step A19]

In this step, compound (25a) is chlorinated to give compound (26a).

There are no particular limitations on the reaction conditions, and compound (25a) can be reacted with phosphorus oxychloride, phosphorus pentachloride, or a mixture thereof in a solvent or in the absence of a solvent at a temperature of 0° C. to 150° C. Solvents include, for example, toluene, acetonitrile, and dichloroethane.

[Step A20]

In this step, compound (26a) is reacted with compound (7a) to give compound (19a).

The reaction can be conducted under the same conditions as used in [Step A6] of production method A.
Production Method B 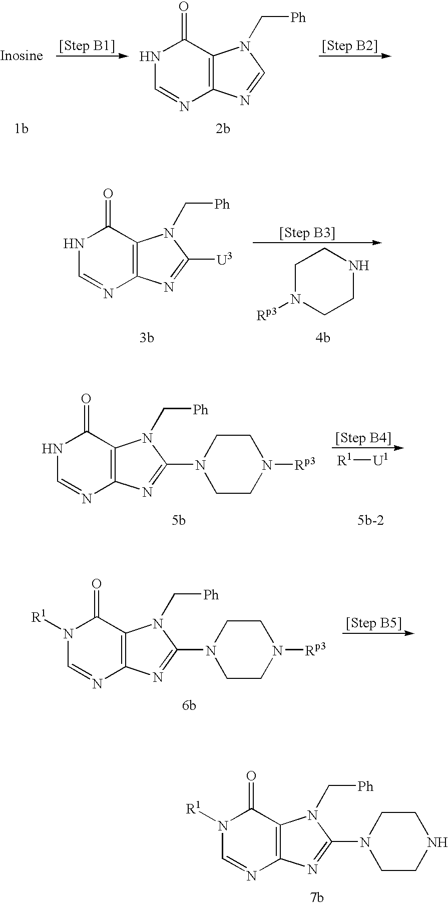
[Step B1]

In this step, compound (1b) is benzylated and the sugar chain is cleaved to give compound (2b).

There are no particular limitations on the reaction conditions. Compound (2b) can be obtained by reacting compound (1b) with benzyl bromide in a solvent such as acetonitrile, N,N-dimethylformamide, N-methylpyrrolidone, dimethyl sulfoxide, 1,4-dioxane, tetrahydrofuran, dimethoxyethane, methanol, or ethanol, at a temperature of 0° C. to 150° C., adding three to ten equivalents of hydrochloric acid, and incubating the mixture at a temperature of 0° C. to 150° C. to cleave the sugar moiety. It is preferable to use one to three equivalents of benzyl bromide.

[Step B2]

In this step, compound (2b) is reacted with a halogenating agent to give compound (3b). The halogenation reaction can be conducted under the same conditions as used in [Step A5] of production method A.

[Step B3]

In this step, compound (3b) is reacted with compound (4b) to give compound (5b). The reaction can be conducted under the same conditions as used in [Step A6] of production method A.

[Step B4]

In this step, compound (5b) is reacted with compound (5b-2) to give compound (6b). The reaction can be conducted under the same condition as used in [Step A2] of production method A.

[Step B5]

In this step, R^{p3} of compound (6b) is removed to give compound (7b). The reaction can be conducted under the same conditions as used in [Step A13] of production method A.

Production Method B-2

Compound (9b) represented by the formula: 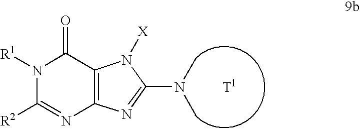
can be obtained by using compound (8b) represented by H-T^{1a}, instead of compound (7a) in [Step A6] of production method A described above, under the same reaction conditions as used in [Step A6], and then appropriately applying [Step A7] to [Step A13] described above.

Compound (10b) represented by the formula: 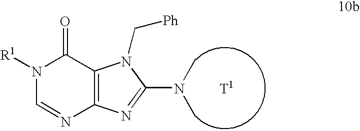
can be obtained by using compound (8b) represented by H-T^{1a}, instead of compound (3b) in [Step B3] of production method B described above, under the same reaction conditions as used in [Step B3], and then appropriately applying [Step B4] to [Step B6] described above. Preferable examples of compound (8b) include piperidin-3-yl carbamic acid t-butyl ester.
Production Method C 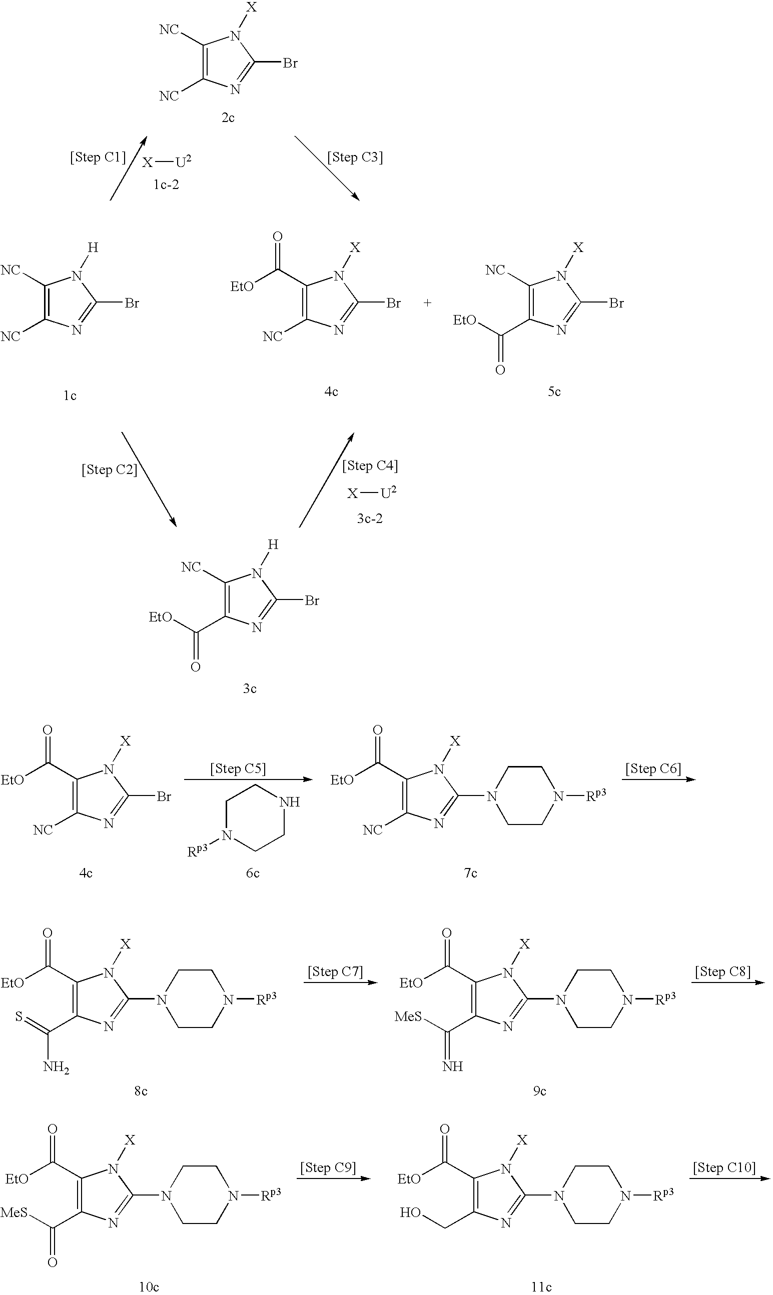 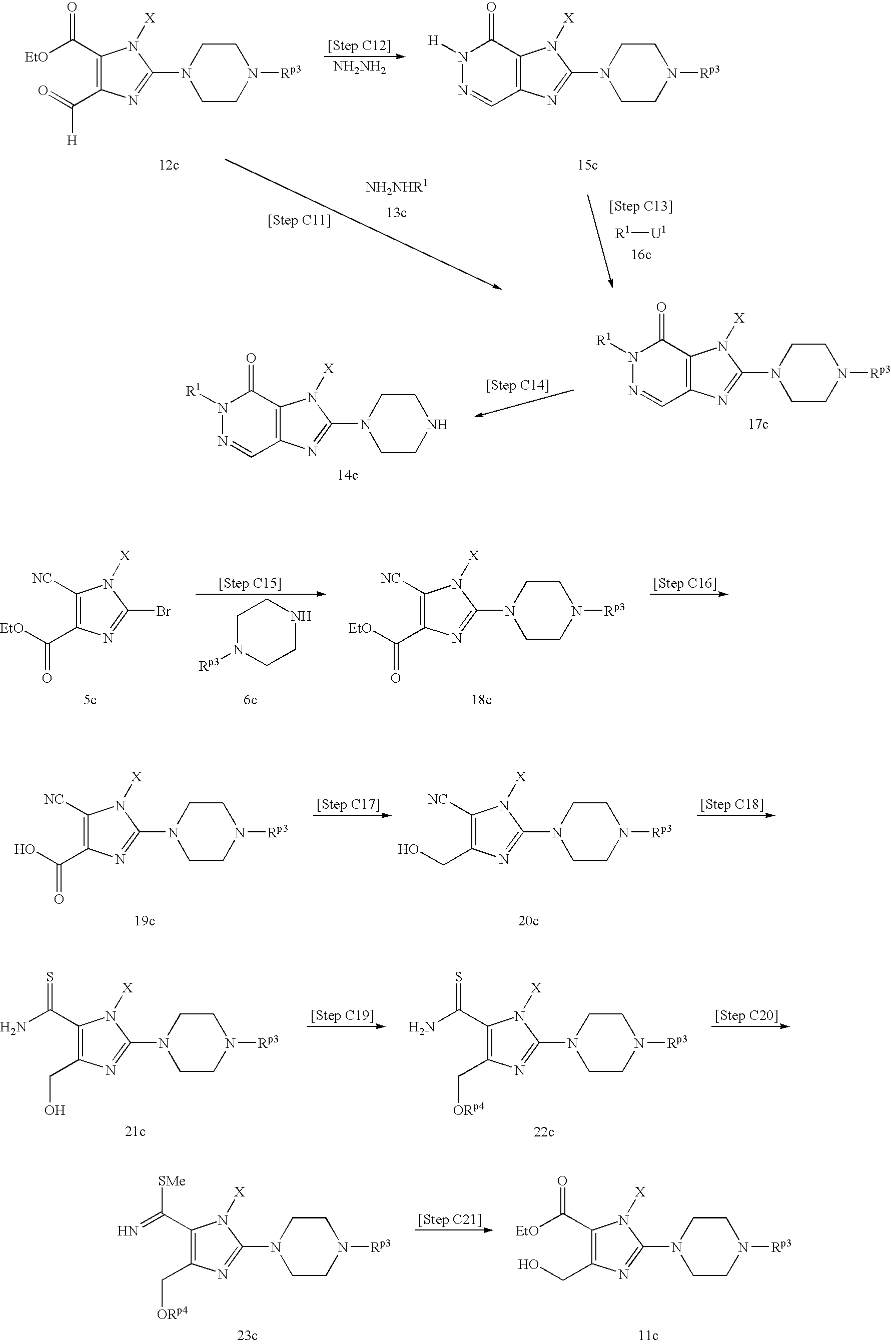
[Step C1]

In this step, compound (1c) is reacted with compound (lc-2) to give compound (2c). The reaction can be conducted under the same conditions as used in [Step A4] of production method A.

[Step C2]

In this step, compound (1c) is reacted with ethanol to give compound (3c).

Compound (3c) can be obtained, for example, by heating an ethanol solution of compound (2c) under reflux in the presence of an acid such as sulfuric acid or hydrochloric acid. However, the reaction conditions are not limited thereto. In this reaction, it is preferable to use one to two equivalents of acid.

[Step C3]

In this step, compound (2c) is reacted with ethanol to give compounds (4c) and (5c). The reaction can be conducted under the same conditions as used in [Step C2] of production method C.

[Step C4]

In this step, compound (3c) is reacted with compound (3c-2) to give compounds (4c) and (5c). The reaction can be conducted under the same conditions as used in [Step A4] of production method A.

[Step C5]

In this step, compound (4c) is reacted with compound (6c) to give compound (7c). The reaction can be conducted under the same conditions as used in [Step A6] of production method A.

[Step C6]

In this step, compound (7c) is thioamidated to give compound (8c). Solvents for the reaction include methanol, ethanol, N,N-dimethylformamide, N-methylpyrrolidone, 1,4-dioxane, tetrahydrofuran, and dimethoxyethane. Thioamidation reagents include ammonium sulfide, sodium sulfide, and hydrogen sulfide. It is preferable to use two to ten equivalents of thioamidation reagent. When hydrogen sulfide is used as the thioamidation reagent, the reaction is carried out in the presence of a base such as triethylamine or N,N-diisopropylethylamine. The reaction can be conducted at a temperature ranging from 0° C. to 150° C.

[Step C7]

In this step, compound (8c) is reacted with a methylating reagent to give compound (9c). Methylating reagents include trimethyl oxonium tetrafluoroborate, methyl sulfate, methyl iodide, and trimethyl phosphite. It is preferable to use 1.0 to 1.5 equivalents of methylating reagent.

When trimethyl oxonium tetrafluoroborate is used as the methylating reagent, compound (9c) can be obtained by carrying out the reaction in a halogenated solvent such as dichloromethane, at a temperature ranging from 0° C. to 50° C.

When methyl sulfate, methyl iodide, or trimethyl phosphite is used as the methylating reagent, compound (9c) can be obtained by carrying out the reaction in the presence of a base such as potassium carbonate, triethylamine, or N,N-diisopropylethylamine. In this case, it is preferable to use 1.0 to 1.5 equivalents of base. Solvents for the reaction include acetone, N,N-dimethylformamide, N-methylpyrrolidone, 1,4-dioxane, tetrahydrofuran, and dimethoxyethane. The reaction can be performed at a temperature ranging from 0° C. to 100° C.

[Step C8]

In this step, compound (9c) is hydrolyzed to give compound (10c).

There are no particular limitations on the reaction conditions for hydrolysis. The reaction can be carried out in a mixed solvent of ethanol and water in the presence of an acid such as sulfuric acid, hydrochloric acid, or p-toluenesulfonic acid, at a temperature ranging from 0° C. to 80° C. In this case, it is preferable to use five to 50 equivalents of the acid.

When R^{p3} is a group, such as a t-butoxycarbonyl group, which is removed under the above-described condition, the protecting group should be reintroduced. There are no particular limitations on the reaction conditions for introducing this protecting group. When R^{p3} is a t-butoxycarbonyl group, the reaction can be carried out using a reagent such as t-butyl dicarbonate in a solvent such as dichloromethane, chloroform, N,N-dimethylformamide, or tetrahydrofuran, in the presence of a base such as pyridine, 4-aminopyridine, triethylamine, or N,N-diisopropylethylamine, at a temperature ranging from 0° C. to 80° C. In this case, it is preferable to use two to three equivalents of base.

[Step C9]

In this step, compound (10c) is reacted with a reducing agent to give compound (1 Ic).

There are no particular limitations on the reaction conditions for the reduction. The reaction can be achieved by reacting compound (10c) with hydrogen in the presence of Raney nickel in a solvent such as benzene, ethanol, 2-propanol, or acetone, at a temperature ranging from 0° C. to 50° C., or alternatively reacting compound (10c) with a reducing agent such as sodium borohydride, in a solvent such as methanol, ethanol, or 2-methyl-2-propanol, or in a mixed solvent of water and tetrahydrofuran at a temperature ranging from 0° C. to 50° C., or alternatively reacting compound (10c) with a reducing agent such as sodium borohydride, in the presence of one to five equivalents of a mercury salt such as mercuric acetate in a solvent such as methanol, ethanol, or 2-methyl-2-propanol at a temperature ranging from 0° C. to 50° C. It is preferable to use two to three equivalents of a reducing agent.

[Step C10]

In this step, compound (11c) is subjected to an oxidation reaction to give compound (12c).

When an oxidant such as manganese dioxide, pyridinium chlorochromate, or pyridinium dichromate is used in the oxidation reaction, compound (12c) can be obtained by carrying out the reaction in a solvent such as dichloromethane or chloroform, at a temperature ranging from 20° C. to 80° C. Alternatively, compound (12c) can also be obtained by carrying out the reaction under standard conditions for the oxidation of a primary alcohol to aldehyde, such as Swern oxidation. It is preferable to use five to 20 equivalents of an oxidant.

[Step C11]

In this step, compound (12c) is reacted with compound (13c) to give compound (17c). In this case, it is preferable to use two to ten equivalents of compound (13c).

Compound (17c) can be obtained, for example, by combining compounds (12c) and (13c) in a solvent such as methanol, ethanol, 1-methyl-2-pyrrolidone, 1,4-dioxane, tetrahydrofuran, or dimethoxyethane, or in the absence of solvent, and reacting the mixture at a temperature of 20° C. to 150° C. However, the reaction conditions are not limited thereto.

[Step C12]

In this step, compound (12c) is reacted with hydrazine to give compound (15c). The reaction can be conducted under the same conditions as used in [Step C11] of production method C. It is preferable to use two to ten equivalents of hydrazine.

[Step C13]

In this step, a substitution reaction is carried out using compound (15c) and compound (16c) to give compound (17c). The reaction can be conducted under the same conditions as used in [Step A2] of production method A. It is preferable to use one to three equivalents of compound (16c).

[Step C14]

In this step, R^{p3} of compound (17c) is removed to give compound (14c). The reaction can be conducted under the same conditions as used in [Step A13] of production method A.

[Step C15]

In this step, compound (5c) is reacted with compound (6c) to give compound (18c). The reaction can be conducted under the same conditions as used in [Step A6] of production method A.

[Step C16]

In this step, compound (18c) is hydrolyzed to give compound (19c).

There are no particular limitations on the reaction conditions for the hydrolysis. For example, compound (19c) can be obtained by incubating compound (18c) in the presence of a base at a temperature ranging from 0° C. to 100° C.

Solvents for the reaction include methanol, ethanol, tetrahydrofuran, water, or mixtures thereof. Bases include lithium hydroxide, sodium hydroxide, and potassium hydroxide. It is preferable to use 1 to 2 equivalents of a base.

[Step C17]

In this step, compound (19c) is reacted with a reducing agent to give compound (20c). The reduction can be achieved under a standard condition for the reduction of carboxylic acid to methyl alcohol.

Reducing agents include borane derivatives such as borane-tetrahydrofuran complex and borane-methyl sulfide complex, and sodium borohydride. It is preferable to use 5 to 30 equivalents of a reducing agent.

When a borane derivative is used as a reducing agent, compound (20c) can be obtained by carrying out the reaction using a solvent such as 1,4-dioxane, tetrahydrofuran, or dimethoxyethane at a temperature ranging from −78° C. to 35° C.

Alternatively, when sodium borohydride is used as a reducing agent, first, compound (19c) is reacted with an activator such as isobutyl chloroformate, at a temperature ranging from −78° C. to 20° C., then reacted with a reducing agent such as sodium borohydride at a temperature ranging from −78° C. to 35° C., to obtain compound (20c). Solvents for the reaction include 1,4-dioxane, tetrahydrofuran, and dimethoxyethane.

[Step C18]

In this step, compound (20c) is thioamidated to give compound (21c). The reaction can be conducted under the same conditions as used in [Step C6] of production method C.

[Step C19]

In this step, compound (21c) is reacted with a silylating agent in the presence of a base to give compound (22c).

Solvents for the reaction include dichloromethane, N,N-dimethylformamide, 1,4-dioxane, tetrahydrofuran, and dimethoxyethane. Bases include imidazole, pyridine, 4-dimethylaminopyridine, triethylamine, and N,N-diisopropylethylamine. Silylating agents include t-butyldimethylchlorosilane, and t-butylchlorodiphenylsilane. It is preferable to use 1.0 to 1.5 equivalents of base and 1.0 to 1.5 equivalent of silylating agent. The reaction can be conducted at a temperature ranging from 0° C. to 80° C.

[Step C20]

In this step, compound (22c) is methylated to give compound (23c).

The reaction can be conducted under the same conditions as used in [Step C7] of production method C.

[Step C21]

In this step, compound (23c) is hydrolyzed to give compound (24c).

There are no particular limitations on the reaction conditions for the hydrolysis. Compound (24c) can be obtained by carrying out the reaction in a mixed solvent of ethanol and water in the presence of an acid such as sulfuric acid, hydrochloric acid, orp-toluenesulfonic acid, at a temperature ranging from 50° C. to 100° C.

When such a reaction results in removal of —RP3, —NH— is re-protected through a protection reaction. Specifically, for example, when R^{p3} is a t-butoxycarbonyl group, the reaction can be carried out using a reagent such as t-butyl dicarbonate, in a solvent such as dichloromethane, chloroform, N,N-dimethylformamide, or tetrahydrofuran, in the presence of a base such as pyridine, 4-aminopyridine, triethylamine, or N,N-diisopropyl ethylamine, at a temperature ranging from 0° C. to 80° C. However, the reaction is not limited thereto.
Production Method D 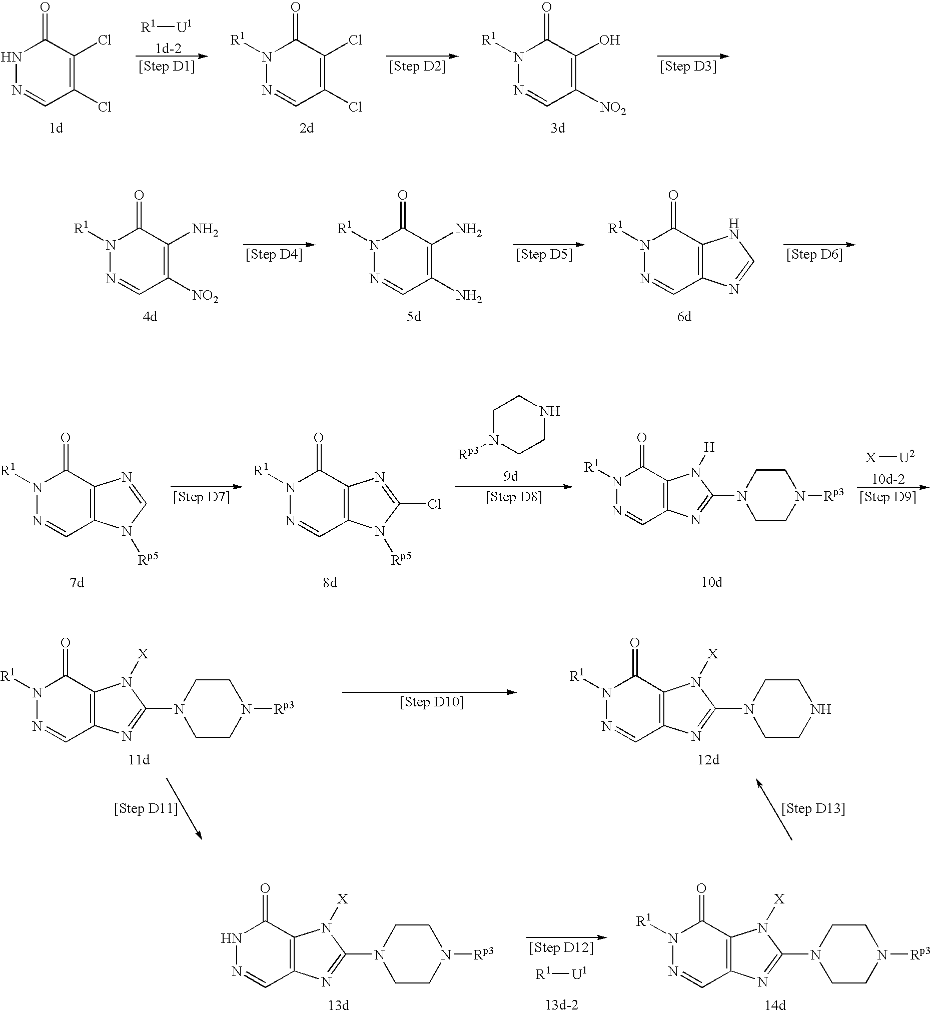
[Step D1]

In this step, compound (1d) is reacted with compound (1d-2) to give compound (2d).

Specifically, compound (1d-2) includes, for example, alkyl halides such as iodomethane, iodoethane, iodopropane, benzyl bromide, 2-bromoacetophenone, chloromethyl benzyl ether, and bromoacetonitrile; alkenyl halides such as allyl bromide and 1-bromo-3-methyl-2-butene; and alkynyl halides such as propargyl bromide and 1-bromo-2-butyne. It is preferable to use one to 1.5 equivalents of compound (1d-2).

Solvents for the reaction include N,N-dimethylformamide, N-methylpyrrolidone, tetrahydrofuran, 1,2-dimethoxyethane, 1,4-dioxane, and dichloromethane. The reaction can be carried out in the presence or absence of a base. Bases to be used in the reaction include 1,8-diazabicyclo[5,4,0]undecene, triethylamine, N,N-diisopropylethylamine, and sodium hydride. In this case, it is preferable to use one to 1.5 equivalents of the base. The reaction can be conducted at a temperature ranging from 0° C. to 150° C.

[Step D2]

In this step, compound (2d) is reacted with a nitrite salt to give compound (3d).

Solvents for the reaction include a mixed solvent of water and a solvent from N,N-dimethylformamide, N-methylpyrrolidone, tetrahydrofuran, 1,2-dimethoxyethane, and 1,4-dioxane. Nitrite salts include sodium nitrite and potassium nitrite. It is preferable to use three to five equivalents of nitrite. The reaction can be conducted at a temperature ranging from 20° C. to 120° C.

[Step D3]

In this step, compound (3d) is reacted with ammonia to give compound (4d). It is preferable to use 10 to 20 equivalents of ammonia.

The reaction can be carried out in a solvent such as methanol, ethanol, or 1,4-dioxane at a temperature ranging from 20° C. to 200° C.

[Step D4]

In this step, compound (4d) is subjected to catalytic reduction under a hydrogen atmosphere or in the presence of two to three equivalents of hydrazine using a metal catalyst to give compound (5d).

Solvents for the reaction include methanol, ethanol, N,N-dimethylformamide, tetrahydrofuran, 1,2-dimethoxyethane, 1,4-dioxane, water, or a mixed solvent thereof. Metal catalysts include palladium carbon, platinum oxide, and Raney nickel. It is preferable to use a metal catalyst in the amount of 0.5% to 10% by weight. The reaction can be conducted at a temperature ranging from 0° C. to 150° C.

[Step D5]

In this step, compound (5d) is reacted with an orthoformate ester to give compound (6d).

The reaction is carried out in the presence of a carboxylic anhydride such as acetic anhydride. Orthoformate esters include methyl orthoformate, and ethyl orthoformate. It is preferable to use one to 20 times as much orthoformate ester by weight and three to ten equivalents of carboxylic anhydride. The reaction can be conducted at a temperature ranging from 20° C. to 200° C.

[Step D6]

In this step, the NH group at the 1-position of compound (6d) is protected to give compound (7d).

Protecting reagents include N,N-dimethylsulfamoyl chloride, trityl chloride, di-t-butyl dicarbonate, and benzyl bromide. It is preferable to use one to 1.5 equivalents of protecting reagent. Solvents for the reaction include dichloromethane, chloroform, carbon tetrachloride, toluene, N,N-dimethylformamide, and tetrahydrofuran. Bases include pyridine, 4-dimethylaminopyridine, 1,8-diazabicyclo[5,4,0]undecene, triethylamine, and N,N-diisopropylethylamine. In typical cases, it is preferable to use 1.2 equivalents of a base. However, when the protecting reagent is di-t-butyl dicarbonate, 0.005 to 0.1 equivalents of 4-dimethylaminopyridine are preferably used. The reaction can be conducted at a temperature ranging from 20° C. to 200° C.

[Step D7]

In this step, compound (7d) is chlorinated to give compound (8d).

There are no particular limitations on the reaction conditions. For example, the reaction is carried out as follows: Compound (7d) is reacted with a base at a temperature ranging from −100° C. to 20° C., and then a chlorinating reagent is reacted thereto. This reaction produces compound (8d). Compound (8d) can also be obtained by reacting compound (7d) with a base in the presence of a chlorinating reagent. Solvents for the reaction include, for example, diethyl ether, tetrahydrofuran, 1,2-dimethoxyethane, and 1,4-dioxane. Bases include n-butyllithium, t-butyllithium, lithium diisopropylamide, lithium bis(trimethylsilyl)amide, and magnesium diisopropylamide. It is preferable to use one to 1.5 equivalents of base. Chlorinating reagents include hexachloroethane, and N-chloro succinimide. It is preferable to use one to three equivalents of a chlorinating reagent.

[Step D8]

In this step, compound (8d) is reacted with compound (9d) to give compound (10d). The reaction can be conducted under the same conditions as used in [Step A6] of production method A.

[Step D9]

In this step, a substitution reaction is carried out using compound (10d) and compound (10d-2) to give compound (11d). The reaction can be conducted under the same conditions as used in [Step A4] of production method A.

[Step D10]

In this step, R^{p3} of compound (11d) is removed to give compound (12d). The reaction can be conducted under the same conditions as used in [Step A13] of production method A.

[Step D11]

In this step, the group at the 5-position of compound (11d) is obtained by dealkylation to give compound (13d). There are no particular limitations on the reaction conditions for the dealkylation. For example, such a reaction can be achieved as follows:

When R¹ is a benzyloxymethyl group, compound (11d) is reacted with three to ten equivalents of boron tribromide, boron trichloride, or such in a solution such as dichloromethane at a temperature ranging from −100° C. to 20° C. This reaction produces compound (13d).

When such a reaction results in removal of R^{p3}, —NH— is re-protected through a protection reaction. Specifically, for example, when R^{p3} is a t-butoxycarbonyl group, the reaction can be carried out using a reagent such as di-t-butyl dicarbonate, in a solvent such as dichloromethane, chloroform, N,N-dimethylformamide, or tetrahydrofuran, in the presence of a base such as pyridine, 4-aminopyridine, triethylamine, or N,N-diisopropylethylamine, at a temperature ranging from 0° C. to 80° C. However, the reaction is not limited thereto.

[Step D12]

In this step, compound (13d) is reacted with compound (13d-2) to give compound (14d). The reaction can be conducted under the same conditions as used in [Step D1] of production method D.

[Step D13]

In this step, R^{p3} of compound (14d) is removed to give compound (12d). The reaction can be conducted under the same conditions as used in [Step A13] of production method A.

An alternative method for producing compound (11d) is described below: 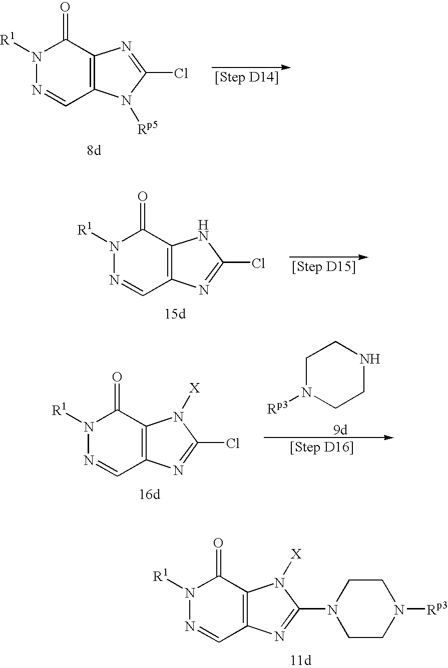
[Step D14]

In this step, compound (8d) is deprotected to give compound (15d).

The deprotection can be achieved under standard reaction conditions depending on the type of protecting group. For example, in the case of a t-butoxycarbonyl group, the deprotection can be achieved by carrying out the reaction using a base such as sodium hydroxide, potassium carbonate, or ammonia, in tetrahydrofuran, N,N-dimethylformamide, methanol, ethanol, water, or a mixed solvent thereof at a temperature ranging from 0° C. to 100° C. When a solvent and a base are added after chlorination in the previous step, the deprotection can be achieved without isolating compound (8d).

[Step D15]

In this step, X is introduced into compound (15d) to give compound (16d). The reaction can be conducted using X-U² under the same conditions as used in [Step A4] of production method A.

An alcohol (X—OH) can be introduced using Mitsunobu's reaction. Specifically, compound (16d) can be obtained by reacting an alcohol (X—OH) with an azodicarboxylic acid dialkyl ester and triphenylphosphine in a solvent such as tetrahydrofuran, at a temperature ranging from −70° C. to 50° C.

[Step D16]

In this step, compound (16d) is reacted with compound (9d) to give compound (11d).

The reaction can be conducted under the same conditions as used in [Step A6] of production method A.

Production Method E

Compound (1e) represented by the formula: 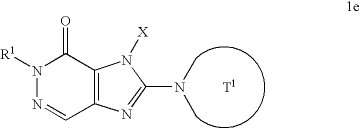
can be obtained by using compound (8b) represented by H-T^{1a}, instead of compound (6c), in [Step C5] or [Step C15] of production method C described above under the same reaction conditions as used in [Step C5], and then appropriately applying [Step C6] to [Step C21] described above.

Compound (1e) represented by the formula: 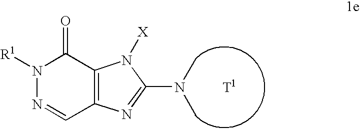
can be obtained by using compound (8b), represented by H-T^{1a}, instead of compound (9d) in [Step D8] of production method D described above, under the same reaction conditions as used in [Step D8], and then appropriately applying [Step D9] to [Step D13] described above.
Production Method F 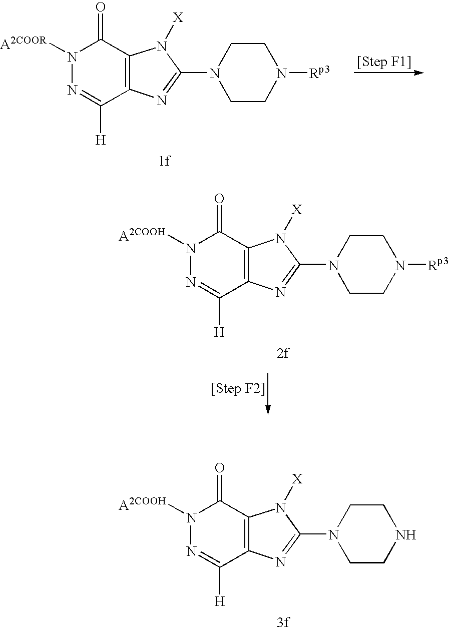
[Step F1]

In this step, the ester group of compound (1f) is hydrolyzed to give compound (2f). The reaction can be conducted under the same conditions as used in [Step C16] of production method C.

[Step F2]

In this step, R^{p3} of compound (2f) is removed to give compound (3f). The reaction can be conducted under the same conditions as used in [Step A13] of production method A.
Production Method G 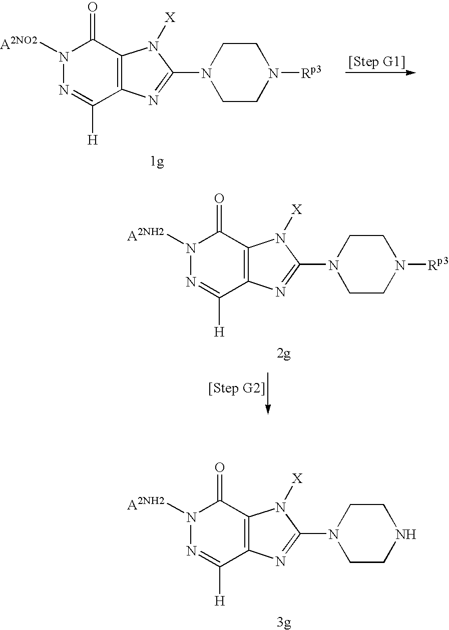
[Step G1]

In this step, the nitro group of compound (1g) is reduced to give compound (2g).

Solvents for the reaction include methanol, ethanol, tetrahydrofuran, water, or mixtures thereof. Reducing agents include iron, tin, and zinc. Catalysts include hydrochloric acid and ammonium salts such as ammonium chloride. The reaction can be conducted at a temperature ranging from 20° C. to 120° C.

[Step G2]

In this step, R^{p3} of compound (2g) is removed to give compound (3g). The reaction can be conducted under the same conditions as used in [Step A13] of production method A.
Production Method H 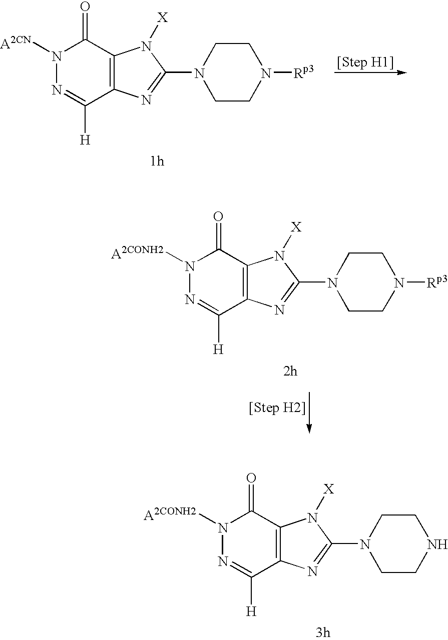
[Step H1]

In this step, the nitrile group of compound (1 h) is hydrolyzed to give compound (2h).

There are no particular limitations on the reaction conditions. For example, the reaction is carried out as follows: Compound (2h) can be obtained by reacting compound (1 h) with hydrogen peroxide in the presence of a base at a temperature ranging from −20° C. to 50° C. Solvents include methanol, ethanol, tetrahydrofuran, water, or a solvent mixture thereof. Bases include ammonia and alkyl amines such as triethylamine.

[Step H2]

In this step, R^{p3} of compound (2h) is removed to give compound (3h). The reaction can be conducted under the same conditions as used in [Step A13] of production method A.
Production Method I 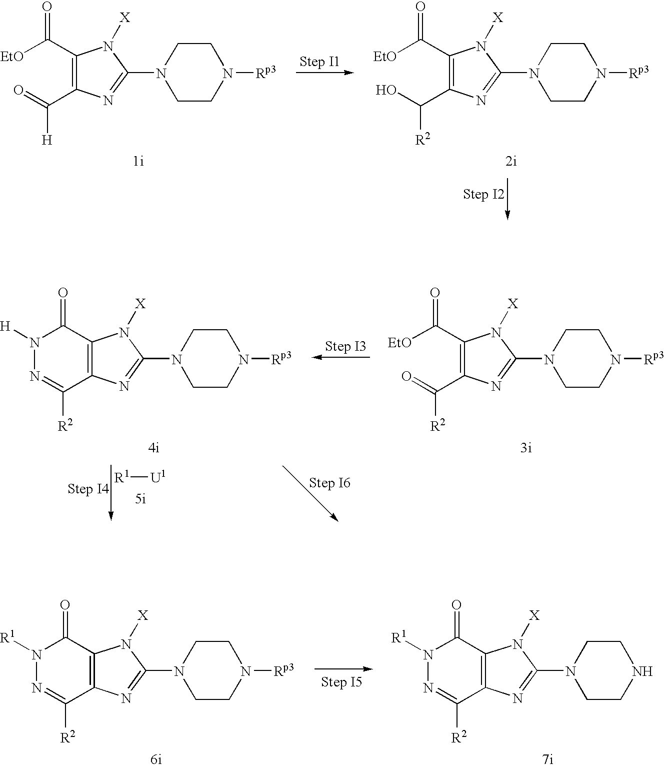
Step I1

In this step, compound (1i) is reacted with an alkyl metal agent or an aryl metal agent to give compound (2i).

There are no particular limitations on the reaction conditions. For example, the reaction is carried out as follows: Compound (1i) may be reacted with an agent such as alkyllithium, aryllithium, alkyl Grignard reagent, or aryl Grignard reagent, in a solvent such as diethyl ether or tetrahydrofuran, at a temperature ranging from −100° C. to 100° C. Alternatively, the compound may be reacted with alkylzinc or arylzinc in a solvent such as N,N-dimethylformamide or 1-methyl-2-pyrrolidone, at a temperature ranging from 0° C. to 50° C.

[Step I2]

In this step, compound (2i) is oxidized to give compound (3i). A typical reagent that is generally used in the oxidation of an alcohol can be used as the oxidant. Specifically, for example, manganese dioxide can be used as the oxidant in a solvent such as dichloromethane or chloroform, at a temperature within the range of 20° C. to 100° C. Alternatively, sulfur trioxide pyridine can be used as the oxidant in a solvent such as dimethyl sulfoxide, at a temperature within the range of 20° C. to 100° C. Alternatively, Dess-Martin periodinane may be used in a solvent such as dichloromethane or chloroform, at a temperature within the range of −50° C. to 50° C.

[Step I3]

In this step, compound (3i) is reacted with hydrazine to give compound (4i). The reaction can be conducted under the same conditions as used in [Step C12] of production method C.

[Step I4]

In this step, a substitution reaction is carried out using compound (4i) and compound (5i) to give compound (6i). The reaction can be conducted under the same conditions as used in [Step A2] of production method A.

[Step I5]

In this step, R^{p3} of compound (6i) is removed to give compound (7i). The reaction can be conducted under the same conditions as used in [Step A13] of production method A.

[Step I6]

In this step, R^{p3} of compound (4i) is removed to give compound (7i) when R¹ of compound (71) is H. The reaction can be conducted under the same conditions as used in [Step A13] of production method A.
Production Method J 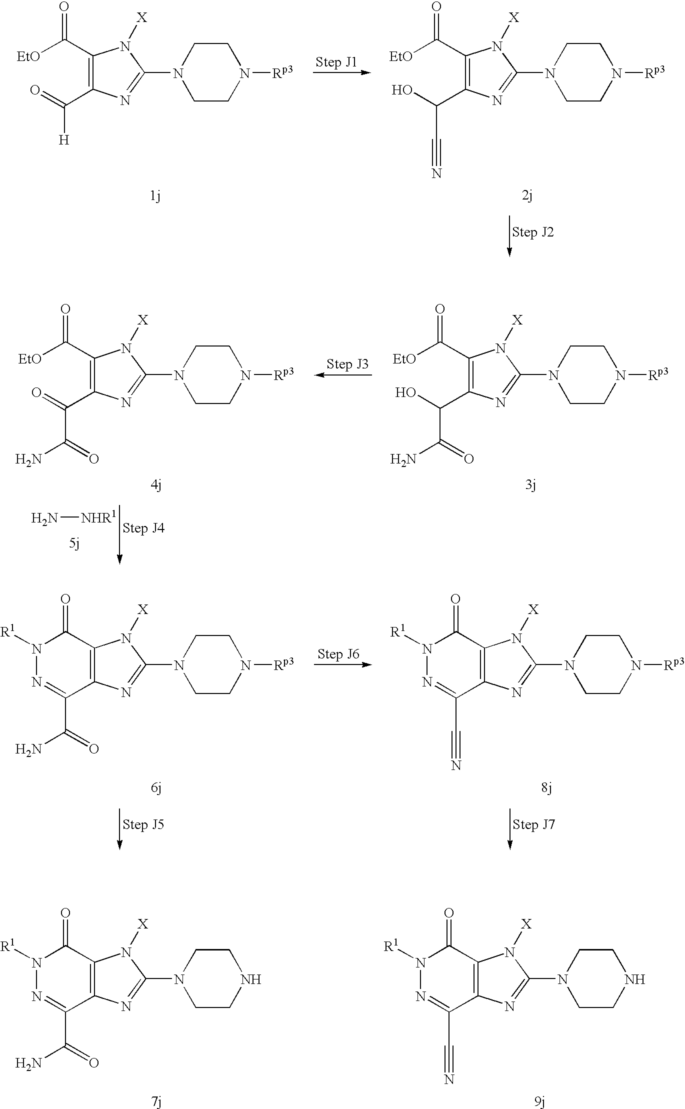
[Step J1]

In this step, compound (1j) is reacted with a cyanidation agent in the presence of a catalyst to give compound (2j).

Cyanidation agents include sodium cyamide and potassium cyamide. Catalysts include acetic acid. Solvents include acetonitrile, for example. The reaction can be conducted at a temperature ranging from 0° C. to 100° C.

[Step J2]

In this step, the nitrile group of compound (2j) is hydrolyzed to give compound (3j). The reaction can be conducted under the same conditions as used in [Step H1] of production method H.

[Step J3]

In this step, the hydroxyl group of compound (3j) is oxidized to give compound (4j). The reaction can be conducted under the same conditions as used in [Step I2] of production method I.

[Step J4]

In this step, compound (4j) is reacted with compound (5j) to give compound (6j). The reaction can be conducted under the same conditions as used in [Step C11] of production method C.

[Step J5]

In this step, R^{p3} of compound (6j) is removed to give compound (7j). The reaction can be conducted under the same conditions as used in [Step A13] of production method A.

[Step J6]

In this step, the carbamoyl group of compound (6j) is dehydrated in the presence of a base to give compound (8j).

Dehydrating agents include, for example, phosphorus oxychloride. Bases include alkyl amines such as triethylamine. Solvents include dichloromethane and chloroform. Alternatively, the reaction can be carried out in the absence of a solvent. The reaction can be conducted at a temperature ranging from 0° C. to 100° C.

[Step J7]

In this step, R^{p3} of compound (8j) is removed to give compound (9j). The reaction can be conducted under the same conditions as used in [Step A13] of production method A.
Production Method K 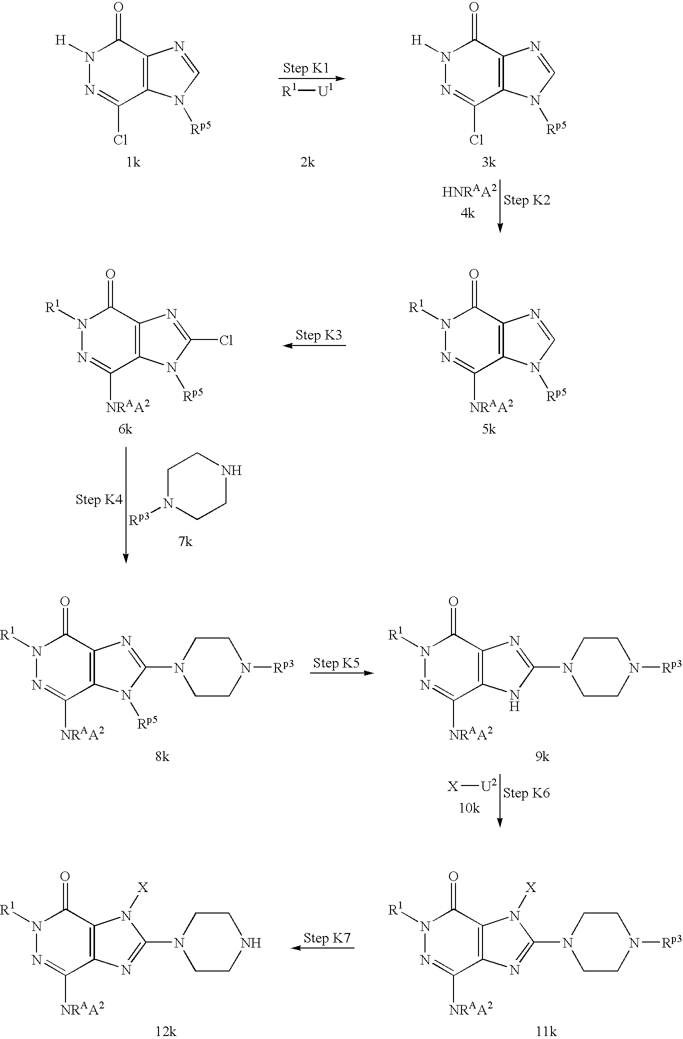
[Step K1]

In this step, a substitution reaction using compound (1k) and compound (2k) is carried out to give compound (3k). The reaction can be conducted under the same conditions as used in [Step A2] of production method A.

[Step K2]

In this step, a substitution reaction using compound (3k) and compound (4k) is carried out to give compound (5k).

Compound (5k) can be obtained, for example, by reacting a mixture of compounds (3k) and (4k) in a solvent such as methanol, ethanol, 1-methyl-2-pyrrolidone, 1,4-dioxane, tetrahydrofuran, or dimethoxyethane, or in the absence of a solvent at a temperature ranging from 20° C. to 200° C. However, the reaction conditions are not limited thereto.

[Step K3]

In this step, compound (5k) is chlorinated to give compound (6k). The reaction can be conducted under the same conditions as used in [Step D7] of production method D.

[Step K4]

In this step, compound (6k) is reacted with compound (7k) to give compound (8k). The reaction can be conducted under the same conditions as used in [Step A6] of production method A.

[Step K5]

In this step, R^{p5} of compound (8k) is removed to give compound (9k).

The deprotection reaction for R^{p5} can be carried out under standard reaction conditions for removing an —NH-protecting group.

For example, when R^{p5} is a benzyl group, the reaction can be achieved using a metal such as lithium or sodium in liquid ammonia at a temperature within the range of −78° C. to −30° C.

[Step K6]

In this step, a substitution reaction using compound (9k) and compound (10k) is carried out to give compound (11k). The reaction can be conducted under the same conditions as used in [Step A4] of production method A.

[Step K7]

In this step, R^{p3} of compound (11k) is removed to give compound (12k). The reaction can be conducted under the same conditions as used in [Step A13] of production method A.
Production Method L 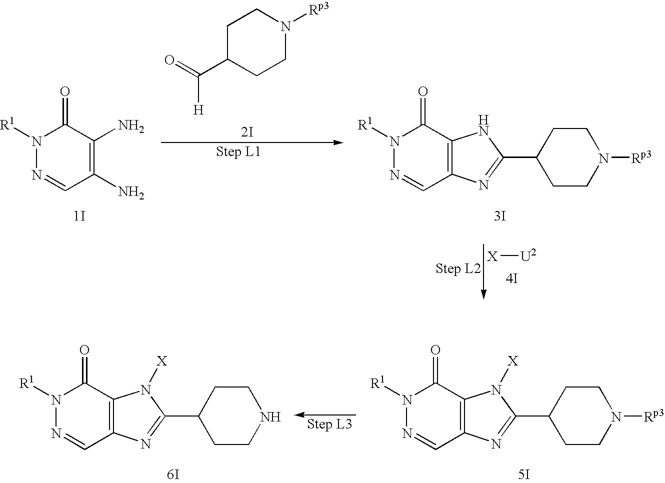
[Step L1]

In this step, compound (1l) is reacted with compound (2l) in the presence of an oxidant to give compound (3l).

Oxidants include salts such as iron (III) chloride. Solvents include methanol, ethanol, and water. The reaction can be conducted at a temperature ranging from 20° C. to 100° C.

When such a reaction results in removal of —R^{p3}, —NH— is re-protected through a protection reaction. Specifically, for example, when Pro3 is a t-butoxycarbonyl group, the reaction can be carried out using a reagent such as di-t-butyl dicarbonate, in a solvent such as dichloromethane, chloroform, N,N-dimethylformamide, or tetrahydrofuran, in the presence of a base such as pyridine, 4-aminopyridine, triethylamine, or N,N-diisopropylethylamine, at a temperature ranging from 0° C. to 80° C. However, the reaction is not limited thereto.

[Step L2]

In this step, compound (3l) is reacted with compound (4l) to give compound (5l). The reaction can be conducted under the same conditions as used in [Step A4] of production method A.

[Step L3]

In this step, R^{p3} of compound (51) is removed to give compound (61). The reaction can be conducted under the same conditions as used in [Step A13] of production method A.
Production Method M 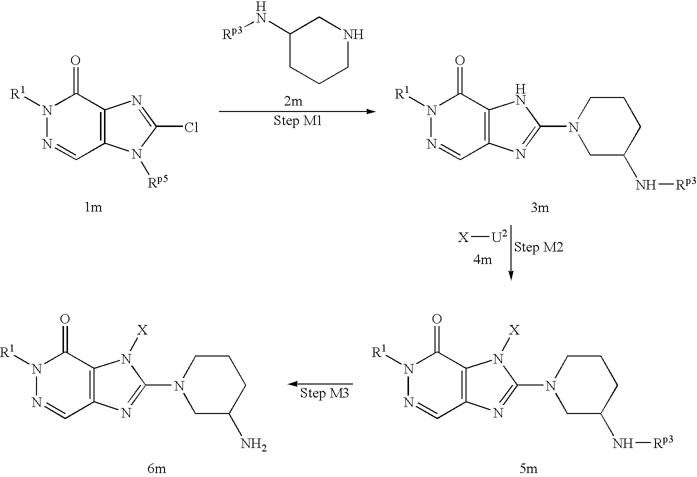
[Step M1]

In this step, compound (1m) is reacted with compound (2m) to give compound (3m). The reaction can be conducted under the same conditions as used in [Step A6] of production method A.

[Step M2]

In this step, compound (3m) is reacted with compound (4m) to give compound (5m). The reaction can be conducted under the same conditions as used in [Step A4] of production method A.

[Step M3]

In this step, R^{p3} of compound (5m) is removed to give compound (6m). The reaction can be conducted under the same conditions as used in [Step A13] of production method A.
Production Method N 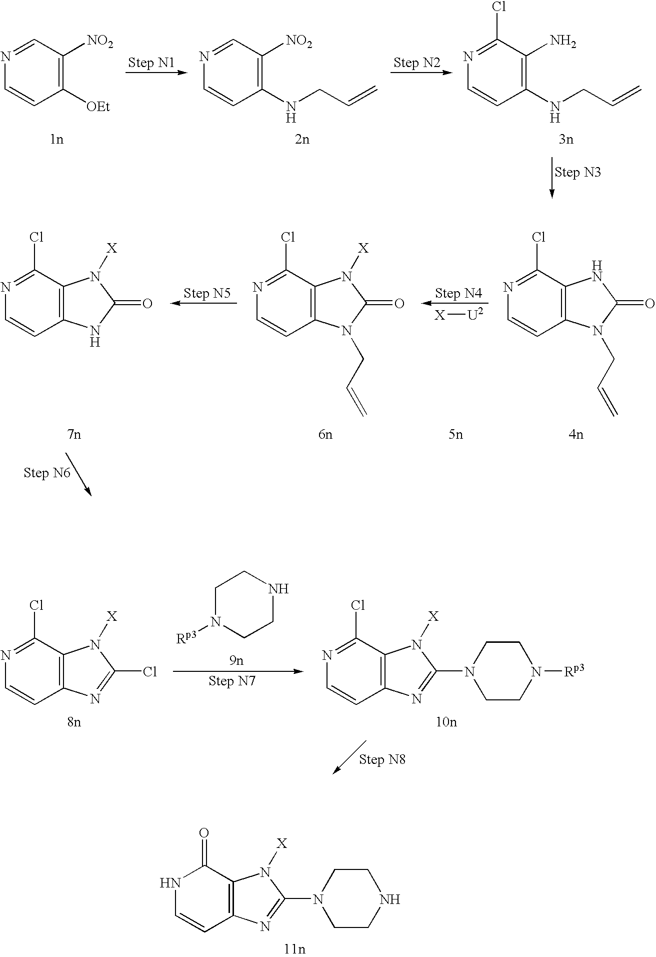
[Step N1]

In this step, compound (1n) is reacted with allylamine to give compound (2n).

The reaction can be conducted at a temperature ranging from 20° C. to 150° C. Solvents for the reaction include methanol, ethanol, water, and mixtures of these solvents.

[Step N2]

In this step, compound (2n) is reduced while being chlorinated to give compound (3n).

Reducing agents include tin salts such as tin chloride. Solvents include concentrated hydrochloric acid. The reaction can be conducted at a temperature ranging from 20° C. to 150° C.

[Step N3]

In this step, compound (3n) is reacted with N,N′-disuccinimidyl carbonate to give compound (4n).

The reaction can be achieved using a solvent such as acetonitrile or tetrahydrofuran. The reaction can be conducted at a temperature ranging from 20° C. to 100° C.

[Step N4]

In this step, compound (4n) is reacted with compound (5n) to give compound (6n). The reaction can be conducted under the same conditions as used in [Step A4] of production method A.

[Step N5]

In this step, the allyl group is removed from compound (6n) to give compound (7n).

Compound (7n) can be obtained, for example, by reacting compound (6n) with osmic acid and sodium periodate in a solvent such as tetrahydrofuran, 1,4-dioxane, 1,2-dimethoxyethane, or water at a temperature ranging from 20° C. to 100° C. However, the reaction conditions are not limited to this example.

[Step N6]

In this step, compound (7n) is chlorinated to give compound (8n).

There are no particular limitations on the reaction conditions. The reaction can be conducted under standard reaction conditions used for chlorination. Compound (8n) can be obtained, for example, by using a reagent such as phosphorus pentachloride in a solvent such as phosphorus oxychloride, at a temperature of 0° C. to 150° C.

[Step N7]

In this step, compound (8n) is reacted with compound (9n) to give compound (10n). The reaction can be conducted under the same conditions as used in [Step A6] of production method A.

[Step N8]

In this step, R^{p3} of compound (10n) is removed to give compound (11n). The reaction can be conducted under the same conditions as used in [Step A13] of production method A.
Production Method O 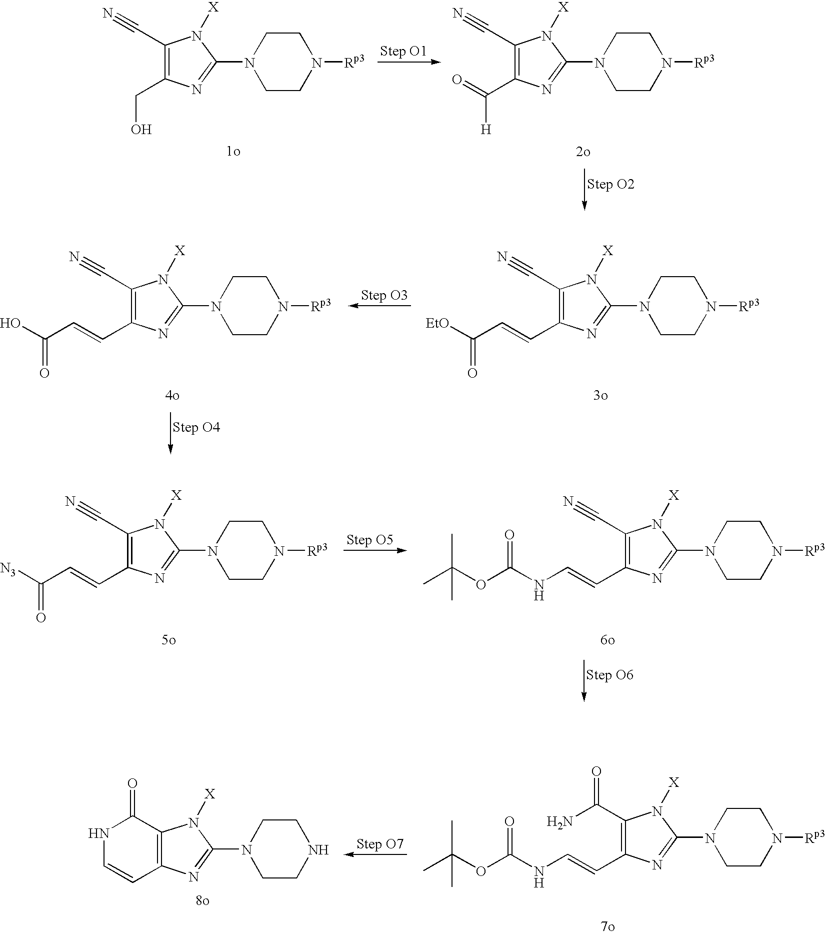
[Step O1]

In this step, the hydroxyl group of compound (1o) is oxidized to give compound (2o). The reaction can be conducted under the same conditions as used in [Step I2] of production method I.

[Step O2]

In this step, compound (20) is reacted with ethyl diethylphosphonoacetate in the presence of a base to give compound (3o).

Bases include sodium hydride and lithium diisopropylamide. Solvents include, for example, tetrahydrofuran and N,N-diformamide. The reaction can be conducted at a temperature ranging from 0° C. to 100° C.

[Step O3]

In this step, the ester of compound (3o) is hydrolyzed to give compound (4o). The reaction can be conducted under the same conditions as used in [Step C16] of production method C.

[Step O4]

In this step, compound (4o) is reacted with diphenylphosphoryl azide in the presence of a base to give compound (5o).

Solvents for the reaction include toluene, t-butanol, tetrahydrofuran, and dichloromethane. Bases include tertiary amines such as triethylamine and diisopropylethylamine. The reaction can be conducted at a temperature ranging from −50° C. to 50° C.

[Step O5]

In this step, compound (5o) is rearranged to give compound (6o).

The reaction can be achieved in t-butanol at a temperature ranging from 50° C. to 100° C.

[Step O6]

In this step, the nitrile group of compound (6o) is hydrolyzed to give compound (7o). The reaction can be conducted under the same conditions as used in [Step H1] of production method H.

[Step O7]

In this step, compound (7o) is reacted with an acid to give compound (8o).

Acids include hydrochloric acid, sulfuric acid, and trifluoroacetic acid. Solvents include methanol, ethanol, 1,4-dioxane, water, and mixtures of these solvents. The reaction can be conducted at a temperature ranging from 0° C. to 50° C.
Production Method P 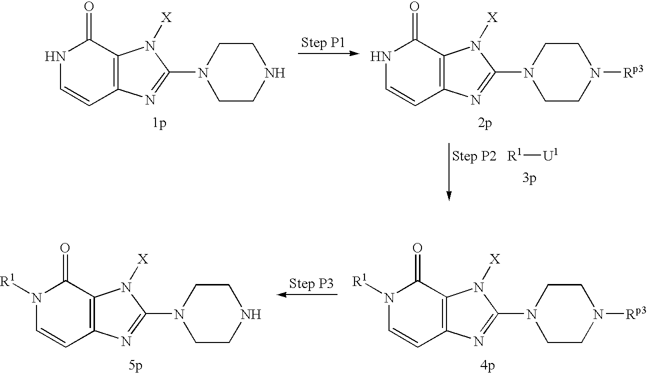
[Step P1]

In this step, compound (1p) is protected to give compound (2p).

A typical NH group-protecting reagent that is generally used in protecting NH groups can be used as an NH group-protecting reagent. For example, when R^{p3} is a t-butoxycarbonyl group, the reaction can be achieved at a temperature ranging from 0° C. to 80° C. using a reagent such as di-t-butyl dicarbonate, in a solvent such as dichloromethane, chloroform, N,N-dimethylformamide, or tetrahydrofuran, in the presence of a base such as pyridine, 4-aminopyridine, triethylamine, or N,N-diisopropylethylamine.

[Step P2]

In this step, compound (2p) is reacted with compound (3p) to give compound (4p). The reaction can be conducted under the same conditions as used in [Step A2] of production method A.

[Step P3]

In this step, R^{p3} of compound (4p) is removed to give compound (5p). The reaction can be conducted under the same conditions as used in [Step A13] of production method A.
Production Method Q 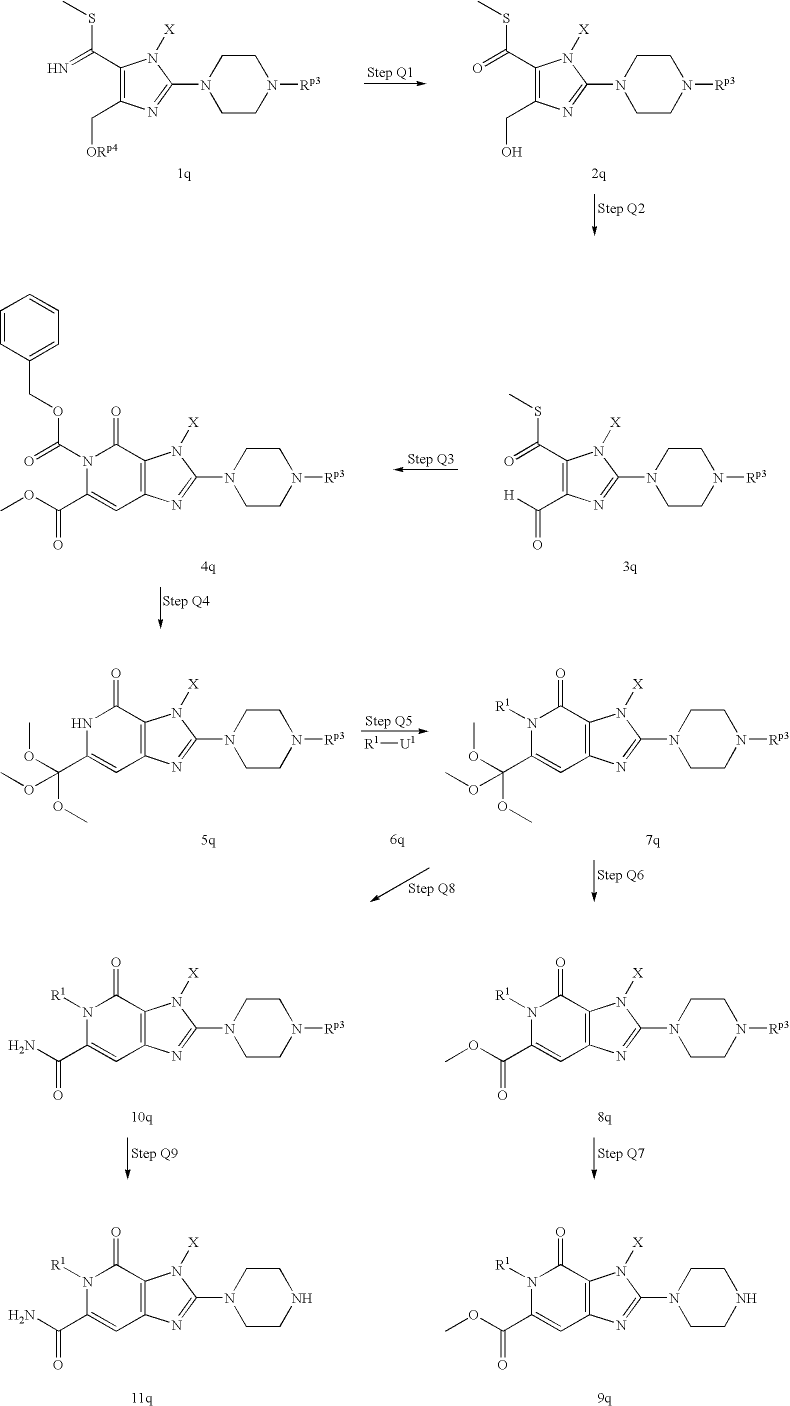
[Step Q1]

In this step, compound (1q) is hydrolyzed to give compound (2q).

Reaction solvents include tetrahydrofuran, methanol, and ethanol. Acids include inorganic acids such as hydrochloric acid and sulfuric acid. The reaction can be conducted at a temperature ranging from 0° C. to 100° C.

[Step Q2]

In this step, the hydroxyl group of compound (2q) is oxidized to give compound (3q). The reaction can be conducted under the same conditions as used in [Step I2] of production method I.

[Step Q3]

In this step, compound (3q) is reacted with methyl benzyloxycarbonylamino(dimethoxyphosphoryl)acetate in the presence of a base to give compound (4q).

Bases include sodium hydride, potassium t-butoxide, and 8-diazabicyclo[5.4.0]-7-undecene. Solvents include dichloromethane, tetrahydrofuran, and N,N-dimethylformamide. The reaction can be conducted at a temperature ranging from 0° C. to 100° C.

[Step Q4]

In this step, compound (4q) is reacted with sodium methoxide to give compound (5q).

Methanol can be used as solvent. The reaction can be conducted at a temperature ranging from 0° C. to 80° C.

[Step Q5]

In this step, compound (5q) is reacted with compound (6q) to give compound (7q). The reaction can be conducted under the same conditions as used in [Step A2] of production method A.

[Step Q6]

In this step, compound (7q) is reacted with an acid to give compound (8q). The reaction can be conducted under the same conditions as used in [Step O7] of production method O.

[Step Q7]

In this step, R^{p3} of compound (8q) is removed to give compound (9q). The reaction can be conducted under the same conditions as used in [Step A13] of production method A.

[Step Q8]

In this step, compound (7q) is reacted with ammonia to give compound (10q).

Reaction solvents include methanol, ethanol, and water. The reaction can be conducted at a temperature ranging from 20° C. to 150° C.

[Step Q9]

In this step, R^{p3} of compound (10q) is removed to give compound (11q). The reaction can be conducted under the same conditions as used in [Step A13] of production method A.

For example, compounds of formula (I) indicated above in which Z¹ is —NR²— and Z² is a carbonyl group, are produced by the method of European Patent Application No. 1338595 (A2).

The methods indicated above are representative methods for producing compound (I) of the present invention. The starting compounds and various reagents to be used in the methods for producing compounds of the present invention may be salts or hydrates, or solvates depending on the type of starting materials, solvents to be used, or such, and are not limited as long as they do not inhibit the reactions. The type of solvent to be used depends on the types of starting compounds, reagents to be used, or such, and is not limited as long as it does not inhibit the reactions and dissolves starting materials to some extent. When compound (I) of the present invention is obtained in a free form, such a compound can be converted to a salt or a hydrate, which is a possible form of compound (I) described above, according to a conventional method.

When compound (I) of the present invention is obtained as a salt or a hydrate, such a product can be converted to a free form of compound (I) described above according to a conventional method.

In addition, various isomers of compound (I) of the present invention (for example, geometric isomers, enantiomers on the basis of asymmetric carbon, rotamers, stereoisomers, and tautomers) can be purified and isolated by typical isolation means, for example, including recrystallization, diastereomer salt method, enzyme-based separation, and various chromatographic methods (for example, thin layer chromatography, column chromatography, and gas chromatography).

Compounds of the present invention, salts thereof, or hydrates thereof, can be formulated into tablets, powders, particles, granules, coated tablets, capsules, syrups, troches, inhalants, suppositories, injections, ointments, eye ointments, eye drops, nasal drops, ear drops, epithem, lotions, etc. by conventional methods.

Such formulation can be achieved by using typical diluting agents, binders, lubricants, colorants, flavoring agents, and if required, stabilizers, emulsifiers, absorbefacients, surfactants, pH modulators, preservatives, antioxidants, etc., and materials commonly used as ingredients of pharmaceutical preparations according to conventional methods.

For example, an oral preparation can be produced by combining a compound of the present invention or a pharmaceutically acceptable salt thereof with a diluting agent, and if required, a binder, a disintegrating agent, a lubricant, a colorant, a flavoring agent, or such, and formulating the mixture into powders, particles, granules, tablets, coated tablets, capsules, or the like according to conventional methods. Examples of the materials include, for example, animal and vegetable oils such as soya bean oil, beef tallow, and synthetic glyceride; hydrocarbons such as liquid paraffin, squalane, and solid paraffin; ester oils such as octyldodecyl myristate and isopropyl myristate; higher alcohols such as cetostearyl alcohol and behenyl alcohol; silicon resins; silicone oils; surfactants such as polyoxyethylene fatty acid ester, sorbitan fatty acid ester, glycerol fatty acid ester, polyoxyethylene sorbitan fatty acid ester, polyoxyethylene hydrogenated castor oil, and polyoxyethylene polyoxypropylene block co-polymer; water-soluble polymers such as hydroxyethyl cellulose, poly-acrylic acid, carboxyvinyl polymer, polyethylene glycol, polyvinylpyrrolidone, and methyl cellulose; lower alcohols such as ethanol and isopropanol; polyhydric alcohols such as glycerol, propylene glycol, dipropylene glycol, and sorbitol; sugars such as glucose and sucrose; inorganic powder such as anhydrous silicic acid, magnesium aluminum silicate, and aluminum silicate; and pure water. Diluting agents include, for example, lactose, corn starch, white sugar, glucose, mannitol, sorbitol, crystal cellulose, and silicon dioxide. Binders include, for example, polyvinyl alcohol, polyvinyl ether, methyl cellulose, ethyl cellulose, gum arabic, tragacanth, gelatin, shellac, hydroxypropyl methyl cellulose, hydroxypropyl cellulose, polyvinylpyrrolidone, polypropylene glycol-polyoxyethylene block co-polymer, and meglumine. Disintegrating agents include, for example, starch, agar, gelatin powder, crystalline cellulose, calcium carbonate, sodium bicarbonate, calcium citrate, dextrin, pectin, and calcium carboxymethyl cellulose. Lubricants include, for example, magnesium stearate, talc, polyethylene glycol, silica, and hydrogenated vegetable oil. Colorants include those pharmaceutically acceptable. Flavoring agents include cocoa powder, peppermint camphor, aromatic powder, peppermint oil, Borneo camphor, and cinnamon powder.

Tablets and granules may be coated with sugar, or if required, other appropriate coatings can be made. Solutions to be administered, such as syrups or injectable preparations, can be formulated by combining a compound of the present invention or a pharmaceutically acceptable salt thereof with a pH modulator, a solubilizing agent, an isotonizing agent, or such, and if required, with an auxiliary solubilizing agent, a stabilizer, or the like, according to conventional methods.

Methods for producing an external preparation are not limited and such preparations can be produced by conventional methods. Specifically, various materials typically used for producing pharmaceuticals, quasi drugs, cosmetics, and such can be used as base materials for the external formulation. Specifically, base materials to be used include, for example, animal and vegetable oils, mineral oils, ester oils, waxes, higher alcohols, fatty acids, silicone oils, surfactants, phospholipids, alcohols, polyhydric alcohols, water-soluble polymers, clay minerals, and pure water. Furthermore, external preparations of the present invention can contain, as required, pH modulators, antioxidants, chelating agents, antibacterial/antifungal agents, coloring matters, odoriferous substances, etc. But this does not limit the type of base materials that are to be used in an external preparation of the present invention. If required, the preparation may contain differentiation inducers, blood flow improving agents, antimicrobial agents, antiphlogistics, cell activators, vitamins, amino acids, humectants, keratolytic agents, etc. The amount of base materials listed above is adjusted within a concentration range used for producing typical external preparations.

When a compound of the present invention, or a salt thereof, or a hydrate thereof is administered, the forms of a compound are not limited and a compound can be given orally or parenterally by a conventional method. For example, a compound can be administered as dosage forms such as tablets, powders, granules, capsules, syrups, troches, inhalants, suppositories, injections, ointments, eye ointments, eye drops, nasal drops, ear drops, epithems, and lotions. The dose of a pharmaceutical of the present invention can be selected appropriately based on symptom severity, age, sex, weight, forms of compounds, types of salts, specific types of diseases, etc.

The dose varies depending on a patient's disease, symptom severity, age and sex, drug susceptibility, etc. A pharmaceutical agent of this invention is administered once or several times at a dose of approximately 0.03 to approx. 1000 mg/adult/day, preferably 0.1 to 500 mg/adult/day, and more preferably 0.1 to 100 mg/adult/day. An injection can be given at a dose of approximately 1 μg/kg to approx. 3000 μg/kg, preferably approximately 3 μg/kg to approx. 1000 μg/kg.

All prior-art documents cited herein are incorporated herein by reference.

### BRIEF DESCRIPTION OF THE DRAWINGS

FIG. 1 is a graph comparing changes in EAE symptoms over time in immunized mice after administration of test compound 1X, control mice (methylcellulose solution-administrated group), and normal mice (non-immunized mice).

FIG. 2 is a graph comparing changes in EAE symptoms over time in immunized mice after administration of test compound 2X or 3X and control mice (methylcellulose solution-administrated group).

### BEST MODE FOR CARRYING OUT THE INVENTION

Compounds of the present invention can be produced, for example, by the methods described in the Examples below. However, the compounds of the present invention are under no circumstances to be construed as being limited to the specific examples described below.

### PRODUCTION EXAMPLE 1

### t-Butyl 4-[1-(2-butynyl)-6-methyl-7-oxo-6,7-dihydro-1H-imidazo[4,5-d]pyridazin-2-yl]piperazin-1-carb oxylate

### (a) t-Butyl 5-methyl-4-oxo-4,5-dihydroimidazo[4,5-d]pyridazine-1-carboxylate

A mixture consisting of 1.0 g of 5-methyl-3,5-dihydroimidazo[4,5-d]pyridazin-4-one, 16 mg of 4-dimethylaminopyridine, 1.6 g of di-t-butyl dicarbonate, and 5 ml of tetrahydrofuran was stirred at room temperature overnight. Then, a 0.5-ml tetrahydrofuran solution containing 300 mg of di-t-butyl dicarbonate was added to the solution, and the resulting mixture was stirred at room temperature for three hours. 5 ml of t-butyl methyl ether was added to the reaction mixture, and the mixture was cooled with ice. The resulting crystals were collected by filtration to give 1.63 g of the title compound.

¹H-NMR(CDCl₃)

δ 1.72 (s, 9H) 3.93 (s, 3H) 8.38 (s, 1H) 8.54 (s, 1H)

### (b) 2-Chloro-5-methyl-1,5-dihydroimidazo[4,5-d]pyridazin-4-one

8.4 ml of lithium hexamethyldisilazide (1.0 M tetrahydrofuran solution) was added dropwise over one hour to a 300-ml tetrahydrofuran solution containing 1.68 g of t-butyl 5-methyl-4-oxo-4,5-dihydroimidazo[4,5-d]pyridazine-1-carboxylate and 4.15 g of hexachloroethane under a nitrogen atmosphere at 0° C. The resulting mixture was stirred for 30 minutes. 2N ammonia water was added to the solution, and the mixture was stirred for three hours. Then, the reaction solution was concentrated to 50 ml, and washed with 20 ml of t-butyl methyl ether. The solution was acidified with concentrated hydrochloric acid. The resulting precipitate was collected by filtration, and washed successively with 10 ml of water and 10 ml of t-butyl methyl ether. Thus, 1.03 g of the title compound was obtained.

¹H-NMR(DMSO-d6)

δ 1.45 (s, 9H) 3.72 (s, 3H) 8.33 (s, 1H)

### (c) 3-(2-Butynyl)-2-chloro-5-methyl-3,5-dihydroimidazo[4,5-d]pyridazin-4-one

7.72 g of 2-chloro-5 methyl-1,5-dihydroimidazo[4,5-d]-pyridazin-4-one was suspended in 400 ml of tetrahydrofuran under a nitrogen atmosphere, and 14.22 g of triphenylphosphine and 3.85 g of 2-butyn-1-ol were added thereto. The resulting mixture was cooled to 0° C. A 100-ml tetrahydrofuran solution containing 12.55 g of azodicarboxylic acid di-t-butyl ester was added dropwise, and the reaction mixture was stirred for three hours. The reaction mixture was concentrated under reduced pressure. 50 ml of dichloromethane and 50 ml of trifluoroacetic acid were added to the residue, and the mixture was stirred for 15 hours. The reaction mixture was concentrated under reduced pressure. The resulting residue was dissolved in 400 ml of ethyl acetate, and washed with a 200 ml of a 5N aqueous sodium hydroxide solution. The aqueous layer was extracted with 100 ml of ethyl acetate. The organic layers were combined together, dried over magnesium sulfate, and concentrated under reduced pressure. The resulting residue was purified by silica gel column chromatography. Thus, 8.78 g of the title compound was obtained from the fraction eluted with hexane-ethyl acetate (4:1).

¹H-NMR(CDCl₃)

δ 1.82 (t, J=2.3 Hz, 3H) 3.87 (s, 3H) 5.32 (q, J=2.3 Hz, 2H) 8.19 (s, 1H)

### (d) t-Butyl 4-[1-(2-butynyl)-6-methyl-7-oxo-6,7-dihydro-1H-imidazo[4,5-d]pyridazin-2-yl]piperazine-1-car boxylate

5 ml of 1-methyl-2-pyrrolidone was added to a mixture consisting of 1.183 g of 3-(2-butynyl)-2-chloro-5-methyl-3,5-dihydroimidazo[4,5-d]pyridazin-4-one, 0.829 g of potassium carbonate, and 1.395 g of t-butyl piperazine-1-carboxylate under a nitrogen atmosphere. The resulting mixture was heated at 130° C. for 6 hours. The reaction mixture was cooled, and 50 ml of water was added thereto. Then, the mixture was extracted with 100 ml of ethyl acetate. The organic layer washed twice with 50 ml of water and then with 50 ml of an aqueous solution saturated with sodium chloride. The organic layer was dried over magnesium sulfate, and concentrated under reduced pressure. The resulting residue was purified by silica gel column chromatography. Thus, 1.916 g of the title compound was obtained from the fraction eluted with hexane-ethyl acetate (1:4).

¹H-NMR(CDCl₃)

δ 1.52 (s, 9H) 1.83 (t, J=2.3 Hz, 3H) 3.38-3.42 (m, 4H) 3.61-3.64 (m, 4H) 3.85 (s, 3H) 5.09 (q, J=2.3 Hz, 2H) 8.13 (s, 1H)

### PRODUCTION EXAMPLE 2

### t-Butyl 4-[7-(2-butvnl)-2,6-dichloro-7H-purin-8-yl]piperazine-1-carboxylate

### (a) 7-(2-Butynyl)-3-methyl-3,7-dihydropurine-2,6-dione

55.3 ml of 1-bromo-2-butyne and 84.9 g of anhydrous potassium carbonate were added to a mixture of 100 g of 3-methyl xanthine [CAS No. 1076-22-8] and 1000 ml of N,N-dimethylformamide. The resulting mixture was stirred at room temperature for 18 hours. 1000 ml of water was added to the reaction solution, and the mixture was stirred at room temperature for 1 hour. The resulting white precipitate was collected by filtration. The white solid washed with water and then t-butyl methyl ether. Thus, 112 g of the title compound was obtained.

¹H-NMR(DMSO-d6)

δ 1.82 (t, J=2.2 Hz, 3H) 3.34 (s, 3H) 5.06 (q, J=2.2 Hz, 2H) 8.12 (s, 1H) 11.16 (br.s, 1H)

### (b) 7-(2-Butynyl)-8-chloro-3-methyl-3,7-dihydropurine-2,6-dione

112 g of 7-(2-butynyl)-3-methyl-3,7-dihydropurine-2,6-dione was dissolved in 2200 ml of N,N-dimethylformamide, and 75.3 g of N-chlorosuccinimide was added thereto. The resulting mixture was stirred at room temperature for five hours. 2200 ml of water was added to the reaction solution, and the mixture was stirred at room temperature for 1.5 hour. The white precipitate was collected by filtration, and the white solid washed with water and, with t-butyl methyl ether. Thus, 117 g of the title compound was obtained.

¹H-NMR(DMSO-d6)

δ 1.78 (t, J=2.0 Hz, 3H) 3.30 (s, 3H) 5.06 (q, J=2.0 Hz, 2H) 11.34 (br.s, 1H)

### (c) 7-(2-Butynyl)-2,6,8-trichloro-7H-purine

A mixture of 2.52 g of 7-(2-butynyl)-8-chloro-3-methyl-3,7-dihydropurine-2,6-dione and 100 ml of phosphorus oxychloride was stirred at 120° C. for 14 hours. After the reaction mixture had been cooled, 4.15 g of phosphorus pentachloride was added to the solution. The resulting mixture was stirred at 120° C. for 24 hours. After the reaction solution had been cooled to room temperature, the solvent was evaporated under reduced pressure. The residue was dissolved in tetrahydrofuran. The solution was poured into a saturated sodium bicarbonate solution, and the mixture was extracted with ethyl acetate. The resulting organic layer was washed with water, then saturated brine, and was then concentrated under reduced pressure. The residue was purified by silica gel column chromatography (ethyl acetate:hexane=1:3) to give 2.40 g of the title compound.

¹H-NMR(CDCl₃)

δ 1.82 (t, J=2.4 Hz, 3H) 5.21 (q, J=2.4 Hz, 2H)

### (d) t-Butyl 4-[7-(2-butvnl)-2,6-dichloro-7H-purin-8-yl]piperazine-1-carboxylate

A mixture of 2.4 g of 7-(2-butynyl)-2,6,8-trichloro-7H-purine, 1.46 g of sodium bicarbonate, 2.43 g of t-butyl piperazine-1-carboxylate, and 45 ml of acetonitrile was stirred at room temperature for 2 hours and 20 minutes. Then, 0.73 g of sodium bicarbonate and 1.21 g of t-butyl piperazine-1-carboxylate were added, and the resulting mixture was stirred at room temperature for 1 hour. The reaction mixture was extracted with ethyl acetate-water, and the organic layer washed with 1N hydrochloric acid, dried over anhydrous magnesium sulfate, and then concentrated under reduced pressure. The residue was triturated with diethyl ether. The crystals were collected by filtration, and washed with diethyl ether. Thus, 3.0 g of the title compound was obtained as a white solid.

¹H-NMR(DMSO-d6)

δ 1.42 (s, 9H) 1.83 (t, J=2 Hz, 3H) 3.48-3.55 (m, 4H) 3.57-3.63 (m, 4H) 4.89 (q, J=2 Hz, 2H)

### EXAMPLE 1

### Ethyl [7-(2-chlorophenyl)-1-methyl-6-oxo-8-(piperazin-1-yl)-6,7-dihydro-1H-purin-2-yloxy]acetate trifluoroacetate

### (a) [7-Benzyl-2,6-dioxo-1,2,6,7-tetrahydropurin-3-yl]methyl 2,2-dimethylpropionate

8.66 g of 7-benzylxanthine was dissolved in 300 ml of N,N-dimethylformamide, and 1.57 g of sodium hydride and 7.7 ml of chloromethyl pivalate were added thereto. The resulting mixture was stirred at room temperature overnight. The reaction solution was diluted with ethyl acetate, and washed with water and 1N hydrochloric acid. The organic layer was dried over anhydrous magnesium sulfate, then filtered. The solvent was evaporated under reduced pressure. The residue was purified by silica gel column chromatography. Thus, 2.66 g of the title compound was obtained from the fraction eluted with hexane-ethyl acetate (1:1).

¹H-NMR(CDCl₃)

δ 1.18 (s, 9H) 5.45 (s, 2H) 6.06 (s, 2H) 7.34-7.39 (m, 5H) 7.58 (s, 1H) 8.18 (s, 1H).

### (b) [7-Benzyl-1-methyl-2,6-dioxo-1,2,6,7-tetrahydropurin-3-yl]methyl 2,2-dimethylpropionate

2.66 g of [7-benzyl-2,6-dioxo-1,2,6,7-tetrahydropurin-3-yl]methyl 2,2-dimethylpropionate was dissolved in 30 ml of N,N-dimethylformamide, and 1.6 g of potassium carbonate and 1 ml of methyl iodide were added thereto. The mixture was stirred at room temperature overnight. The reaction mixture was diluted with ethyl acetate, and washed with water and 1N hydrochloric acid. The organic layer was dried over anhydrous magnesium sulfate, then filtered. The solvent was evaporated under reduced pressure. The residue was triturated with toluene. Thus, 2.16 g of the title compound was obtained.

¹H-NMR(CDCl₃)

δ 1.18 (s, 9H) 3.41 (s, 3H) 5.49 (s, 2H) 6.11 (s, 2H) 7.26-7.39 (m, 5H) 7.57 (s, 1H).

### (c) [1-Methyl-2,6-dioxo-1,2,6,7-tetrahydropurin-3-yl]methyl 2,2-dimethylpropionate

2.349 g of [7-benzyl-1-methyl-2,6-dioxo-1,2,6,7-tetrahydropurin-3-yl]methyl 2,2-dimethylpropionate was dissolved in 100 ml of acetic acid, and 1 g of 10% palladium carbon was added thereto. The mixture was stirred under a hydrogen atmosphere at room temperature overnight. The reaction mixture was filtered and concentrated to give 1.871 g of the title compound.

¹H-NMR(CDCl₃)

δ 1.19 (s, 9H) 3.48 (s, 3H) 6.17 (s, 2H) 7.83 (s, 1H).

### (d) [7-(2-Chlorophenyl)-1-methyl-2,6-dioxo-1,2,6,7-tetrahydropurin-3-yl]methyl 2,2-dimethylopropionate

1.60 g of [1-methyl-2,6-dioxo-1,2,6,7-tetrahydropurin-3-yl]methyl 2,2-dimethylpropionate, 1.83 g of 2-chlorophenylboronic acid, and 1.5 g of copper (II) acetate were suspended in 30 ml of N,N-dimethylformamide, and 3 ml of pyridine was added thereto. The mixture was stirred at room temperature for three days. The reaction mixture was filtered through a short column filled with silica gel, and the filtrate was diluted with ethyl acetate. The organic layer washed with 1N hydrochloric acid, water, and saturated saline, then dried over anhydrous magnesium sulfate and subsequently filtered. The filtrate was concentrated. The residue was suspended in ether, and the suspension was filtered. The filtrate was purified by silica gel column chromatography. Thus, 724 mg of the title compound was obtained from the fraction eluted with hexane-ethyl acetate (3:2).

### (e) t-Butyl 4-[7-(2-chlorophenyl)-3-(2,2-dimethylpropionyloxymethyl)-1-methyl-2,6-dioxo-2,3,6,7-tetrahyd ro-1H-purin-8-yl]piperazine-1-carboxylate

724 mg of [7-(2-chlorophenyl)-1-methyl-2,6-dioxo-1,2,6,7-tetrahydropurin-3-yl]methyl 2,2-dimethylpropionate was suspended in 15 ml of N,N-dimethylformamide, and 760 mg of N-chlorosuccinimide was added thereto. The reaction solution was stirred overnight, and then diluted with ethyl acetate. The solution washed with water and 1N hydrochloric acid, and dried over anhydrous magnesium sulfate, then filtered. The filtrate was concentrated. Thus, 764 mg of [8-chloro-7-(2-chlorophenyl)-1-methyl-2,6-dioxo-1,2,6,7-tetrahydropurin-3-yl]methyl 2,2-dimethylpropionate was obtained. This compound was mixed with 4 g of t-butyl piperazine-1-carboxylate. The mixture was heated at 150° C., and stirred for three hours. Ethyl acetate and water were added to the reaction mixture, and the mixture was separated. The organic layer washed with 1N hydrochloric acid, and dried over anhydrous magnesium sulfate, then filtered. The filtrate was concentrated. The residue was purified by silica gel column chromatography. Thus, 724 mg of the title compound was obtained from the fraction eluted with hexane-ethyl acetate (3:2).

### (f) t-Butyl 4-[7-(2-chlorophenyl)-1-methyl-2,6-dioxo-2,3,6,7-tetrahydro-1H-purin-8-yl]piperazine-1-carbox ylate

t-Butyl 4-[7-(2-chlorophenyl)-3-(2,2-dimethylpropionyloxy methyl)-1-methyl-2,6-dioxo-2,3,6,7-tetrahydro-1H-purin-8-yl]piperazine-1-carboxylate was dissolved in a mixture of 10 ml of methanol and 20 ml of tetrahydrofuran, and 200 mg of sodium hydride was added thereto. The resulting mixture was stirred at room temperature overnight. 1N hydrochloric acid was added to the reaction solution, and the mixture was extracted with ethyl acetate. The organic layer was dried over anhydrous magnesium sulfate, then filtered. The filtrate was concentrated. The residue was suspended in ether and the mixture was filtered. Thus, 450 mg of the title compound was obtained.

¹H-NMR(DMSO-d⁶)

δ 1.35 (s, 9H) 3.04 (s, 3H) 3.06-3.12 (m, 4H) 3.17-3.22 (m, 4H) 7.48 (dt, J=1.6, 7.6 Hz, 1H) 7.53 (dt, J=2.0, 7.6 Hz, 1H) 7.63 (dd, J=2.0, 8.0 Hz, 1H) 7.65 (dd, J=1.6, 8.0 Hz, 1H).

### (g) t-Butyl 4-[2-chloro-7-(2-chlorophenyl)-1-methyl-6-oxo-6,7-dihydro-1H-purin-8-yl]piperazine-1-carboxy late (2-1), and t-butyl 4-[2,6-dichloro-7-(2-chlorophenyl)-7H-purin-8-yl]piperazine-1-carboxylate (g-2)

78 mg of t-butyl 4-[7-(2-chlorophenyl)-1-methyl-2,6-dioxo-2,3,6,7-tetrahydro-1H-purin-8-yl]piperazine-1-carbox ylate was dissolved in 3 ml of phosphorus oxychloride, and the mixture was stirred at 120° C. overnight. The reaction solution was concentrated, and the residue was dissolved in 1 ml of tetrahydrofuran. This solution was poured into a suspension consisting of 50 mg of di-t-butyl dicarbonate, 1 ml of tetrahydrofuran, and 0.5 ml of water containing 100 mg of sodium bicarbonate. The resulting mixture was stirred at room temperature for three hours. The reaction mixture was diluted with ethyl acetate and washed with water. The organic layer was dried over anhydrous magnesium sulfate, then filtered. The filtrate was concentrated, and the residue was purified by silica gel column chromatography. Thus, 16 mg of t-butyl 4-[2,6-dichloro-7-(2-chlorophenyl)-7H-purin-8-yl]piperazine-1-carboxylate was obtained from the fraction eluted with hexane-ethyl acetate (3:2), and 10 mg of t-butyl 4-[2-chloro-7-(2-chlorophenyl)-1-methyl-6-oxo-6,7-dihydro-1H-purin-8-yl]piperazine-1-carboxylate was obtained from the fraction eluted with hexane-ethyl acetate (1:9).

### (h) Ethyl [7-(2-chlorophenyl)-1-methyl-6-oxo-8-(piperazin-1-yl)-6,7-dihydro-1H-purin-2-yloxy]acetate trifluoroacetate

10 mg of t-butyl 4-[2-chloro-7-(2-chlorophenyl)-1-methyl-6-oxo-6,7-dihydro-1H-purin-8-yl]piperazine-1-carboxy late and 10 mg of ethyl glycolate were dissolved in 0.2 ml of N-methylpyrrolidone, and 10 mg of sodium hydride was added thereto. The mixture was stirred at room temperature for two hours. The reaction solution was dissolved in ethyl acetate, and the mixture washed with 1N hydrochloric acid. Thus, 24 mg of t-butyl 4-[7-(2-chlorophenyl)-2-ethoxycarbonylmethoxy-1-methyl-6-oxo-6,7-dihydro-1H-purin-8-yl]pip erazine-1-carboxylate was obtained. 8 mg of this compound was dissolved in trifluoroacetic acid, and the mixture was concentrated. The residue was purified by reverse-phase high performance liquid chromatography (using an acetonitrile-water mobile phase (containing 0.1% trifluoroacetic acid)) to give 2.11 mg of the title compound.

MS m/e (ESI) 447(MH⁺—CF₃COOH)

### EXAMPLE 4

### Methyl 2-[7-(2-butynyl)-1-methyl-6-oxo-8-(piperazin-1-yl)-6,7-dihydro-1H-purin-2-yloxy]phenylacetate trifluoroacetate

### (a) [7-(2-Butynyl)-1-methyl-2,6-dioxo-1,2,6,7-tetrahydropurin-3-yl]methyl 2,2-dimethylpropionate

1.871 g of [1-methyl-2,6-dioxo-1,2,6,7-tetrahydropurin-3-yl]methyl 2,2-dimethylpropionate was dissolved in 30 ml of N,N-dimethylformamide, and 1.5 g of potassium carbonate and 0.7 ml of 2-butynyl bromide were added thereto. The mixture was stirred at room temperature overnight. The reaction mixture was diluted with ethyl acetate, and washed with water and 1N hydrochloric acid. The organic layer was dried over anhydrous magnesium sulfate, then filtered. The solvent was evaporated under reduced pressure, and the residue was purified by silica gel column chromatography. Thus, 2.12 g of the title compound was obtained from the fraction eluted with hexane-ethyl acetate (3:2).

### (b) 7-(2-Butynyl)-1-methyl-3,7-dihydropurine-2,6-dione

The title compound was obtained by treating [7-(2-butynyl)-1-methyl-2,6-dioxo-1,2,6,7-tetrahydropurin-3-yl]methyl 2,2-dimethylpropionate by the same method as used in Example (1f).

¹H-NMR(CDCl₃)

δ 1.91 (t, J=2.4 Hz, 3H) 3.39 (s, 3H) 5.10 (s, 2H) 7.93 (s, 1H) 10.62 (s, 1H).

### (c) t-Butyl 4-[7-(2-butynyl)-1-methyl-2,6-dioxo-2,3,6,7-tetrahydro-1H-purin-8-yl]piperazine-1-carboxylate

The title compound was obtained by treating 7-(2-butynyl)-1-methyl-3,7-dihydropurine-2,6-dione by the same method as used in Example (1e).

¹H-NMR(CDCl₃)

δ 1.48 (s, 9H) 1.83 (t, J=2.4 Hz, 3H) 3.37 (s, 3H) 3.37-3.39 (m, 4H) 3.58-3.60 (m, 4H) 4.87 (s, 2H) 9.68 (s, 1H).

### (d) Methyl 2-[7-(2-butynyl)-1-methyl-6-oxo-8-(piperazin-1-yl)-6,7-dihydro-1H-purin-2-yloxy]phenylacetate trifluoroacetate

8 mg of t-butyl 4-[7-(2-butynyl)-1-methyl-2,6-dioxo-2,3,6,7-tetrahydro-1H-purin-8-yl]piperazine-1-carboxylate and 10 mg of methyl 2-bromophenylacetate were dissolved in 0.2 ml of N,N-dimethylformamide, and 10 mg of potassium carbonate was added thereto. The mixture was stirred at 50° C. overnight. Ethyl acetate was added to the reaction solution, and the mixture washed with water and 1N hydrochloric acid. The organic layer was concentrated. The residue was dissolved in trifluoroacetic acid, and the mixture was concentrated. The residue was purified by reverse-phase high performance liquid chromatography (using an acetonitrile-water mobile phase (containing 0.1% trifluoroacetic acid)) to give 1.07 mg of the title compound.

MS m/e (ESI) 451(MH⁺—CF₃COOH)

### EXAMPLE 9

### Ethyl 2-[7-(2-butynyl)-1-methyl-6-oxo-8-(piperazin-1-yl)-6,7-dihydro-1H-purin-2-yloxy]propionate

Using ethyl 2-bromopropionate instead of methyl 2-bromophenylacetate in Example (4d), trifluoroacetate of the title compound was obtained by the same method as used in Example 4. The compound was purified by chromatography using NH-silica gel (silica gel whose surface had been modified with amino groups: Fuji Silysia Chemical Ltd. NH-DM 2035). Thus, the title compound was obtained from the fraction eluted with ethyl acetate-methanol (20:1).

MS m/e (ESI) 404(MH⁺)

### EXAMPLE 11

### 7-(2-Butynyl)-2-methoxy-1-methyl-8-(piperazin-1-yl)-1,7-dihydropurin-6-one trifluoroacetate

### (a) t-Butyl 4-[7-(2-butynyl)-2-chloro-1-methyl-6-oxo-6,7-dihydro-1H-purin-8-yl]piperazine-1-carboxylate(a-1, and t-butyl 4-[7-(2-butynyl)-2,6-dichloro-7H-purin-8-yl]piperazine-1-carboxylate (a-2)

5.127 g of t-butyl 4-[7-(2-butynyl)-1-methyl-2,6-dioxo-2,3,6,7-tetrahydro-1H-purin-8-yl]piperazine-1-carboxylate was dissolved in 75 ml of phosphorus oxychloride, and then the mixture was stirred at 120° C. overnight. The reaction solution was concentrated, and the residue was dissolved in 50 ml of tetrahydrofuran. This solution was poured into a suspension consisting of 7 g of di-t-butyl dicarbonate, 50 ml of tetrahydrofuran, 100 g of sodium bicarbonate, and 200 ml of water, and the mixture was stirred at room temperature for one hour. The reaction mixture was diluted with ethyl acetate, and the mixture washed with water. The organic layer was dried over anhydrous magnesium sulfate, then filtered. The filtrate was concentrated, and the residue was purified by silica gel column chromatography. Thus, 1.348 g of t-butyl 4-[7-(2-butynyl)-2,6-dichloro-7H-purin-8-yl]piperazine-1-carboxylate [¹H-NMR(CDCl₃) δ 1.50 (s, 9H) 1.87 (t, J=2.4 Hz, 3H) 3.64 (m, 8H) 4.81 (q, J=2.4 Hz, 2H)] was obtained from the fraction eluted with hexane-ethyl acetate (1:1), and 1.238 g of t-butyl 4-[7-(2-butynyl)-2-chloro-1-methyl-6-oxo-6,7-dihydro-1H-purin-8-yl]piperazine-1-carboxylate [¹H-NMR(CDCl₃) δ 1.49 (s, 9H) 1.83 (t, J=2.4 Hz, 3H) 3.42-3.44 (m, 4H) 3.59-3.62 (m, 4H) 3.73 (s, 3H) 4.93 (q, J=2.4 Hz, 2H)] was obtained from the fraction eluted with hexane-ethyl acetate (1:9).

### (b) 7-(2-Butvn l)-2-methoxy-1-methyl-8-(piperazin-1-yl)-1,7-dihydropurin-6-one trifluoroacetate

8 mg of t-butyl 4-[7-(2-butynyl)-2-chloro-1-methyl-6-oxo-6,7-dihydro-1H-purin-8-yl]piperazine-1-carboxylate was dissolved in 0.2 ml of methanol, and 10 mg of sodium hydride was added thereto. The mixture was stirred at room temperature for one hour. 1N hydrochloric acid was added to the reaction solution, and the mixture was extracted with ethyl acetate. The organic layer was concentrated, and the residue was dissolved in trifluoroacetic acid. The mixture was then concentrated, and the residue was purified by reverse-phase high performance liquid chromatography (using an acetonitrile-water mobile phase (containing 0.1% trifluoroacetic acid)) to give 1.72 mg of the title compound.

MS m/e (ESI) 317(MH⁺—CF₃COOH)

### EXAMPLE 13

### Ethyl[7-(2-butynyl)-1-methyl-6-oxo-8-(piperazin-1-yl)-6,7-dihydro-1H-purin-2-yloUx]acetate

### EXAMPLE 14

### [7-(2-Butynyl)-1-methyl-6-oxo-8-(piperazin-1-yl)-6,7-dihydro-1H-purin-2-yloxy]acetic acid Ethyl

[7-(2-butynyl)-1-methyl-6-oxo-8-(piperazin-1-yl)-6,7-dihydro-1H-purin-2-yloxy]acetate trifluoroacetate and [7-(2-butynyl)-1-methyl-6-oxo-8-(piperazin-1-yl)-6,7-dihydro-1H-purin-2-yloxy]acetic acid trifluoroacetate [MS m/e (ESI) 361(MH⁺—CF₃COOH)] were obtained by treating t-butyl 4-[7-(2-butynyl)-2-chloro-1-methyl-6-oxo-6,7-dihydro-1H-purin-8-yl]piperazine-1-carboxylate using ethyl 2-hydroxyacetate, instead of ethanol, by the same method used in Example 11. Ethyl[7-(2-butynyl)-1-methyl-6-oxo-8-(piperazin-1-yl)-6,7-dihydro-1H-purin-2-yloxy]acetate trifluoroacetate was purified by chromatography using NH-silica gel. Thus, ethyl [7-(2-butynyl)-1-methyl-6-oxo-8-(piperazin-1-yl)-6,7-dihydro-1H-purin-2-yloxy]acetate [¹H-NMR(CDCl₃) δ 1.29 (t, J=7.2 Hz, 3H) 1.83 (t, J=2.4 Hz, 3H) 3.02-3.06 (m, 4H) 3.38-3.41 (m, 4H) 3.55 (s, 3H) 4.22 (q, J=7.2 Hz, 2H) 4.90 (q, J=2.4 Hz, 2H) 5.03 (s, 2H); MS m/e (ESI) 389(MH⁺)] was obtained from the fraction eluted with ethyl acetate-methanol (20:1)

### EXAMPLE 16

### Ethyl 1-[7-(2-butynyl)-1-methyl-6-oxo-8-(piperazin-1-yl)-6,7-dihydro-1H-purin-2-yloxy]cyclopropane carboxylate

Using ethyl 1-hydroxycyclopropanecarboxylate instead of the ethyl 2-hydroxyacetate in Example 13, the trifluoroacetate of the title compound was obtained by the same method used in Example 13. The compound was purified by chromatography using NH-silica gel. Thus, the title compound was obtained from the fraction eluted with ethyl acetate-methanol (20:1).

¹H-NMR(CDCl₃)

δ 1.19 (t, J=7.2 Hz, 3H) 1.39-1.42 (m, 2H) 1.67-1.71 (m, 2H) 1.83 (t, J=2.4 Hz, 3H) 3.02-3.05 (m, 4H) 3.37-3.40 (m, 4H) 3.49 (s, 3H) 4.14 (q, J=7.2 Hz, 2H) 4.90 (q, J=2.4 Hz, 2H)

MS m/e (ESI) 415(MH⁺)

### EXAMPLE 82

### 7-(2-Butynyl)-2-cyano-1-methyl-8-(piperazin-1-yl)-1,7-dihydropurin-6-one trifluoroacetate

8 mg of t-butyl 4-[7-(2-butynyl)-2-chloro-1-methyl-6-oxo-6,7-dihydro-1H-purin-8-yl]piperazine-1-carboxylate was dissolved in 0.2 ml of N-methylpyrrolidone, and 10 mg of sodium cyamide was added thereto. The mixture was stirred at 50° C. for one hour. Water was added to the reaction mixture, and the mixture was extracted with ethyl acetate. The organic layer was concentrated to give 14 mg of t-butyl 4-[7-(2-butynyl)-2-cyano-1-methyl-6-oxo-6,7-dihydro-1H-purin-8-yl]piperazine-1-carboxylate. 5 mg of this compound was dissolved in trifluoroacetic acid, and the solution was concentrated. The residue was purified by reverse-phase high performance liquid chromatography (using an acetonitrile-water mobile phase (containing 0.1% trifluoroacetic acid)) to give 4.12 mg of the title compound.

MS m/e (ESI) 312(MH⁺—CF₃COOH)

### EXAMPLE 95

### 7-(2-Butynyl)-2-chloro-8-(piperazin-1-yl)-1,7-dihydropurin-6-one trifluoroacetate

### (a) t-Butyl 4-[7-(2-butynyl)-2-chloro-6-oxo-6,7-dihydro-1H-purin-8-yl]piperazine-1-carboxylate

A mixture consisting of 1.0 g of t-butyl 4-[7-(2-butynyl)-2, 6-dichloro-7H-purin-8-yl]piperazine-1-carboxylate, 580 mg of sodium acetate, and 10 ml of dimethyl sulfoxide was stirred in an oil bath at 80° C. for 24 hours. The reaction solution was extracted with ethyl acetate and water. The organic layer washed with water and then with saturated brine, and then was dried over anhydrous magnesium sulfate, and concentrated under reduced pressure. The residue was purified by silica gel column chromatography using 50% to 70% ethyl acetate/hexane and crystallized with ethyl acetate-hexane to give 800 mg of the title compound.

¹H-NMR(CDCl₃)

δ 1.49 (s, 9H) 1.83 (t, J=2 Hz, 3H) 3.44 (br.s, 4H) 3.56-3.63 (m, 4H) 4.94 (q, J=2 Hz, 2H)

### (b) 7-(2-Butynyl)-2-chloro-8-(piperazin-1-yl)-1,7-dihydropurin-6-one trifluoroacetate

8 mg of t-butyl 4-[7-(2-butynyl)-2-chloro-6-oxo-6,7-dihydro-1H-purin-8-yl]piperazine-1-carboxylate was dissolved in trifluoroacetic acid, and the solution was concentrated. The residue was purified by reverse-phase high performance liquid chromatography (using an acetonitrile-water mobile phase (containing 0.1% trifluoroacetic acid)) to give 3.45 mg of the title compound.

MS m/e (ESI) 307(MH⁺—CF₃COOH)

### EXAMPLE 96

### 2-[7-(2-Butynyl)-2-dimethylamino-6-oxo-8-(piperazin-1-yl)-6,7-dihydropurin-1-ylmethyl]benzo nitrile hydrochloride

### (a) t-Butyl 4-[7-(2-butynyl)-2-chloro-1-(2-cyanobenzyl)-6-oxo-6,7-dihydro-1H-purin-8-yl]piperazine-1-car boxylate

A mixture consisting of 100 mg of t-butyl 4-[7-(2-butynyl)-2-chloro-6-oxo-6,7-dihydro-1H-purin-8-yl]piperazine-1-carboxylate, 60 mg of 2-cyanobenzyl bromide, 68 mg of anhydrous potassium carbonate, and 1 ml of N,N-dimethylformamide was stirred at room temperature for four hours. Ethyl acetate/hexane (1/1) and water were added to the reaction solution. The insoluble material was removed by filtration. The filtrate was extracted with ethyl acetate. The organic layer washed with water and then with saturated brine, dried over anhydrous magnesium sulfate, and then concentrated under reduced pressure. The residue was purified by silica gel column chromatography using 30% to 50% ethyl acetate/hexane to give 50 mg of the title compound.

¹H-NMR(CDCl₃)

δ 1.49 (s, 9H) 1.83 (t, J=2 Hz, 3H) 3.43-3.49 (m, 4H) 3.58-3.64 (m, 4H) 4.95 (q, J=2 Hz, 2H) 5.72 (s, 2H) 7.06 (d, J=8 Hz, 1H) 7.39 (t, J=8 Hz, 1H) 7.51 (t, J=8 Hz, 1H) 7.71 (d, J=8 Hz, 1H)

### (b) t-Butyl 4-[7-(2-butynyl)-1-(2-cyanobenzyl)-2-dimethylamino-6-oxo-6,7-dihydro-1H-purin-8-yl]piperazi ne-1-carboxylate

A mixture consisting of 8 mg of t-butyl 4-[7-(2-butynyl)-2-chloro-1-(2-cyano benzyl)-6-oxo-6,7-dihydro-1H-purin-8-yl]piperazine-1-carboxylate, 20 μl of an aqueous solution of 50% dimethylamine, and 0.2 ml of N,N-dimethylformamide was stirred at room temperature for two hours. The reaction solution was extracted with ethyl acetate and water. The organic layer washed with water and with saturated brine, and concentrated. The residue was separated by silica gel thin-layer chromatography using 70% ethyl acetate/hexane to give 6.5 mg of the title compound.

¹H-NMR(CDCl₃)

δ 1.50 (s, 9H) 1.81 (t, J=2 Hz, 3H) 2.73 (s, 6H) 3.38-3.45 (m, 4H) 3.56-3.64 (m, 4H) 4.91, (q, J=2 Hz, 2H) 5.55 (s, 2H) 7.07 (d, J=8 Hz, 1H) 7.32 (t, J=8 Hz, 1H) 7.46, (t, J=8 Hz, 1H) 7.65 (d, J=8 Hz, 1H)

### (c) 2-[7-(2-Butynyl)-2-dimethylamino-6-oxo-8-(piperazin-1-yl)-6,7-dihydropurin-1-ylmethyl]benzo nitrile hydrochloride

6.5 mg of t-butyl 4-[7-(2-butynyl)-1-(2-cyanobenzyl)-2-dimethylamino-6-oxo-6,7-dihydro-1H-purin-8-yl]piperazine-1-carboxylate was dissolved in 0.5 ml of trifluoroacetic acid, and the mixture was allowed to stand at room temperature for 20 minutes. The reaction solution was concentrated, and the residue was purified by reverse-phase column chromatography using 20% to 80% methanol/water (containing 0.1% concentrated hydrochloric acid) to give 6.4 mg of the title compound.

¹H-NMR(DMSO-d6)

δ 1.76 (s, 3H) 2.69 (s, 6H) 3.28 (br.s, 4H) 3.51 (br.s, 4H) 4.91 (s, 2H) 5.40 (s, 2H) 7.04 (d, J=8 Hz, 1H) 7.43 (t, J=8 Hz, 1H) 7.60 (t, J=8 Hz, 1H) 7.83 (d, J=8 Hz, 1H) 8.90 (br.s, 2H)

### EXAMPLE 115

### 3-(2-Butynyl)-5-methyl-2-(piperazin-1-yl)-3,5-dihydroimidazo[4,5-d]pyridazin-4-one trifluoroacetate

### (a) Ethyl 2-bromo-3-(2-butynyl)-5-cyano-3H-imidazole-4-carboxylate

4.56 ml of sulfuric acid was added to 170 ml of ethanol containing 16.80 g of 2-bromo-1H-imidazole-4,5-dicarbonitrile [CAS No. 50847-09-1], and the mixture was heated under reflux for 48 hours. The solution was cooled, and then 500 ml of ethyl acetate and 200 ml of water were added thereto. The organic layer was dried over anhydrous magnesium sulfate, filtered, and concentrated under reduced pressure. The residue was dissolved in N,N-dimethylformamide, and 14.1 g of potassium carbonate and 8.6 ml of 2-butynyl bromide were added thereto. The mixture was stirred at room temperature for 18 hours. 500 ml of ethyl acetate was added to the solution, and the mixture washed three times with 300 ml of water, and then with 300 ml of a saturated sodium chloride solution. Then, the solution was dried over anhydrous magnesium sulfate, and filtered. The filtrate was concentrated under reduced pressure. The residue was purified by silica gel column chromatography. Thus, 4.09 g of the title compound was obtained from the fraction eluted with hexane-ethyl acetate (9:1).

¹H-NMR(CDCl₃)

δ 1.43 (t, J=7.2 Hz, 3H) 1.81 (s, 3H) 4.47 (q, J=7.2 Hz, 2H) 5.16 (s, 2H)

### (b) t-Butyl 4-[1-(2-butynyl)-4-cyano-5-ethoxycarboxyl-1H-imidazol-2-yl]piperazine-1-carboxylate

4.09 g of ethyl 2-bromo-3-(2-butynyl)-5-cyano-3H-imidazole-4-carboxylate was combined with 7.70 g of t-butyl piperazine-1-carboxylate, and the mixture was heated to 150° C. with stirring for 50 minutes. The reaction mixture was dissolved in toluene. The mixture was purified by silica gel column chromatography. Thus, 4.47 g of the title compound was obtained from the fraction eluted with hexane-ethyl acetate (2:1).

¹H-NMR(CDCl₃)

δ 1.43 (t, J=7.2 Hz, 3H) 1.47 (s, 9H) 1.82 (t, J=2.3 Hz, 3H) 3.08-3.13 (m, 4H) 3.57-3.61 (m, 4H) 4.44 (q, J=7.2 Hz, 2H) 4.89 (q, J=2.3 Hz, 2H)

### (c) t-Butyl 4-[1-(2-butynyl)-5-ethoxycarbonyl-4-thiocarbamoyl-1H-imidazol-2-yl]piperazine-1-carboxylate

5 ml of an aqueous solution of 50% ammonium sulfide was added to a 20-ml ethanol solution containing 0.80 g of t-butyl 4-[1-(2-butynyl)-4-cyano-5-ethoxycarbonyl-1H-imidazol-2-yl]piperazine-1-carboxylate, and the mixture was heated at 60° C. for 14 hours. 100 ml of ethyl acetate and 50 ml of water were added to the mixture, and the organic layer washed successively with 50 ml of water and 50 ml of a saturated sodium chloride solution. The reaction solution was dried over anhydrous magnesium sulfate, then filtered. The filtrate was concentrated under reduced pressure. The residue was purified by silica gel column chromatography. Thus, 0.58 g of the title compound was obtained from the fraction eluted with hexane-ethyl acetate (3:2).

¹H-NMR(CDCl₃)

δ 1.43 (t, J=7.2 Hz, 3H) 1.48 (s, 9H) 1.82 (t, J=2.3 Hz, 3H) 3.12-3.16 (m, 4H) 3.54-3.59 (m, 4H) 4.44 (q, J=7.2 Hz, 2H) 4.89 (q, J=2.3 Hz, 2H) 7.41 (bras, 1H) 8.88 (br.s, 1H)

### (d) t-Butyl 4-[1-(2-butynyl)-5-ethoxycarbonyl-4-methylsulfanylcarbonimidoyl-1H-imidazol-2-yl]piperazine-1-carboxylate

0.235 of trimethyl oxonium tetrafluoroborate was added to a 20-ml dichloromethane solution of 0.58 g of t-butyl 4-[1-(2-butynyl)-5-ethoxycarbonyl-4-thiocarbamoyl-1H-imidazol-2-yl]piperazine-1-carboxylate, and the mixture was stirred at room temperature for 18 hours. 50 ml of dichloromethane was added to the solution, and the mixture washed with 20 ml of a saturated sodium bicarbonate solution. The mixture was dried over anhydrous magnesium sulfate, and concentrated under reduced pressure to give 0.55 g of the title compound.

¹H-NMR(CDCl₃)

δ 1.41 (t, J=7.2 Hz, 3H) 1.47 (s, 9H) 1.81 (t, J=2.3 Hz, 3H) 2.39 (s, 3H) 3.12-3.16 (m, 4H) 3.56-3.59 (m, 4H) 4.42 (q, J=7.2 Hz, 2H) 4.80 (q, J=2.3 Hz, 2H)

### (e) t-Butyl 4-[1-(2-butynyl)-5-ethoxycarbonyl-4-methylsulfanylcarbonyl-1H-imidazol-2-yl]piperazine-1-car boxylate

5 ml of a 2N aqueous solution of hydrochloric acid was added to a 30-ml ethanol solution of 0.55 g of t-butyl 4-[1-(2-butynyl)-5-ethoxycarbonyl-4-methyl sulfanylcarbonimidoyl-1H-imidazol-2-yl]piperazine-1-carboxylate, and the mixture was heated at 60° C. for five hours. After the reaction solution had been concentrated under reduced pressure, 25 ml of ethyl acetate and 1N sodium hydroxide solution were added thereto. The aqueous layer was extracted with 25 ml of ethyl acetate, and the organic layers were combined together. The mixture washed with 10 ml of a saturated sodium chloride solution containing 1 ml of 1N sodium hydroxide solution, and dried over anhydrous magnesium sulfate. The solution was filtered, and the filtrate was concentrated under reduced pressure. The residue was dissolved in 10 ml of dichloromethane, and then 0.10 ml of triethylamine and 0.256 g of di-t-butyl dicarbonate were added thereto. The mixture was stirred at room temperature for 15 hours, and then 25 ml of ethyl acetate was added thereto. The mixture washed successively with 10 ml of 0.1N hydrochloric acid, 10 ml of a saturated sodium bicarbonate solution, and 10 ml of a saturated sodium chloride solution, and then dried over anhydrous magnesium sulfate. The solution was concentrated under reduced pressure. The residue was purified by silica gel column chromatography. Thus, 0.15 g of the title compound was obtained from the fraction eluted with hexane-ethyl acetate (4:1).

¹H-NMR(CDCl₃)

δ 1.43 (t, J=7.1 Hz, 3H) 1.48 (s, 9H) 1.81 (t, J=2.3 Hz, 3H) 2.40 (s, 3H) 3.16-3.20 (m, 4H) 3.55-3.59 (m, 4H) 4.35 (q, J=7.1 Hz, 2H) 4.80 (q, J=2.3 Hz, 2H)

### (f) t-Butyl 4-[1-(2-butynyl)-5-ethoxycarbonyl-4-hydroxymethyl-1H-imidazol-2-yl]piperazine-1-carboxylate

0.187 g of mercury (II) acetate and 0.090 of sodium borohydride were added to 8 ml of an ethanol solution containing 0.265 g of t-butyl 4-[1-(2-butynyl)-5-ethoxycarbonyl-4-methylsulfanyl carbonyl-1H-imidazol-2-yl]piperazine-1-carboxylate at 0° C., and the mixture was stirred at room temperature for four hours. After 0.187 g of mercury (II) acetate and 0.090 of sodium borohydride had been added to the solution, the mixture was stirred at room temperature for 15 hours. 100 ml of ethyl acetate and 50 ml of 0.5N hydrochloric acid were added to the solution, and the organic layer washed successively with 50 ml of water and 50 ml of a saturated sodium chloride solution. The mixture was dried over anhydrous magnesium sulfate, and concentrated under reduced pressure. The residue was purified by silica gel column chromatography. 0.172 g of the starting material was collected from the fraction eluted with hexane-ethyl acetate (4:1). Then, 0.061 g of the title compound was obtained from the fraction eluted with hexane-ethyl acetate (1:4).

¹H-NMR(CDCl₃)

δ 1.42 (t, J=7.1 Hz, 3H) 1.48 (s, 9H) 1.81 (t, J=2.3 Hz, 3H) 3.17-3.21 (m, 4H) 3.41 (t, J=4.8 Hz, 1H) 3.56-3.60 (m, 4H) 4.36 (q, J=7.1 Hz, 2H) 4.75 (d, J=4.8 Hz, 2H) 4.81 (q, J=2.3 Hz, 2H)

### (g) t-Butyl 4-[1-(2-butynyl)-5-ethoxycarbonyl-4-formyl-1H-imidazol-2-yl]piperazine-1-carboxylate

0.120 g of manganese dioxide was added to a 2-ml dichloromethane solution of 0.061 g of t-butyl 4-[1-(2-butynyl)-5-ethoxycarbonyl-4-hydroxymethyl-1H-imidazol-2-yl]piperazine-1-carboxylate, and the mixture was stirred at room temperature for 15 hours. The reaction solution was filtered through celite, and the filtrate was concentrated under reduced pressure. The residue was purified by silica gel column chromatography. Thus, 0.055 g of the title compound was obtained from the fraction eluted with hexane-ethyl acetate (7:3).

¹H-NMR(CDCl₃)

δ 1.42 (t, J=7.1 Hz, 3H) 1.48 (s, 9H) 1.82 (t, J=2.3 Hz, 3H) 3.23-3.26 (m, 4H) 3.55-3.59 (m, 4H) 4.45 (q, J=7.1 Hz, 2H) 4.89 (q, J=2.3 Hz, 2H) 10.36 (s, 1H)

### (h) t-Butyl 4-[1-(2-butynyl)-6-methyl-7-oxo-6,7-dihydro-1H-imidazo[4,5-d]pyridazin-2-yl]piperazine-1-car boxylate

0.05 ml of methylhydrazine was added to a 2,5-ml ethanol solution of 0.055 g of t-butyl 4-[1-(2-butynyl)-5-ethoxycarbonyl-4-formyl-1H-imidazol-2-yl]piperazine-1-carboxylate. The mixture was stirred at 80° C. for 15 hours, and then heated at 130° C. for 14 hours. The reaction solution was concentrated under reduced pressure. Then, the residue was purified by silica gel column chromatography. Thus, 0.035 g of the title compound was obtained from the fraction eluted with hexane-ethyl acetate (1:1).

¹H-NMR(CDCl₃)

δ 1.52 (s, 9H) 1.83 (t, J=2.3 Hz, 3H) 3.38-3.42 (m, 4H) 3.61-3.64 (m, 4H) 3.85 (s, 3H) 5.09 (q, J=2.3 Hz, 2H) 8.13 (s, 1H)

MS m/e (ESI) 387.4(MH⁺)

### (i) 3-(2-Butynyl)-5-methyl-2-(piperazin-1-yl)-3,5-dihydroimidazo[4,5-d]pyridazin-4-one trifluoroacetate

0.4 ml of trifluoroacetic acid was added to a 0.4-ml dichloromethane solution of 0.0351 g of t-butyl 4-[1-(2-butynyl)-6-methyl-7-oxo-6,7-dihydro-1H-imidazo[4,5-d]pyridazin-2-yl]piperazine-1-car boxylate, and the mixture was stirred at room temperature for one hour. The solvent was concentrated. The residue was purified by reverse-phase high performance liquid chromatography (using an acetonitrile-water mobile phase (containing 0.1% trifluoroacetic acid)) to give 0.0295 g of the title compound.

¹H-NMR(CD₃OD)

δ 1.83 (t, J=2.3 Hz, 3H) 3.45-3.49 (m, 4H) 3.65-3.69 (m, 4H) 3.83 (s, 3H) 5.15 (q, J=2.3 Hz, 2H) 8.20 (s, 1H)

MS m/e (ESI) 287.09(MH⁺—CF₃COOH)

### EXAMPLE 116

### 5-Benzyloxymethyl-3-(2-butynyl)-2-(piperazin-1-yl)-3,5-dihydro-imidazo[4,5-d]pyridazin-4-one trifluoroacetate

### (a) 5-Benzyloxymethyl-4-oxo-4,5-dihydroimidazo[4,5-d]pyridazine-1-sulfonic acid dimethylamide

2.08 g of triethylamine, 2.80 g of N,N-dimethyl sulfamoyl chloride, and 0.22 g of 4-dimethylaminopyridine were added to 50 ml of a dichloromethane solution of 3.04 g of 5-benzyloxy methylimmidazo[4,5-d]pyridazin-4-one [CAS NO. 82137-50-6] (R. Paul Gagnier, Michael J. Halat, and Brian A. Otter, Journal of Heterocyclic Chemistry, 21, p 481, 1984), and the mixture was heated under reflux for four hours. 250 ml of ethyl acetate was added to the solution, and the mixture washed successively with 50 ml of an aqueous solution of 1N hydrochloric acid, 50 ml of a saturated sodium bicarbonate solution, and 50 ml of a saturated sodium chloride solution. The mixture was dried over anhydrous magnesium sulfate, and concentrated under reduced pressure. The residue was purified by silica gel column chromatography. Thus, 2.86 g of the title compound was obtained from the fraction eluted with hexane-ethyl acetate (2:3).

¹H-NMR(CDCl₃)

δ 2.98 (s, 6H) 4.77 (s, 2H) 5.74 (s, 2H) 7.30-7.39 (m, 5H) 8.21 (s, 1H) 8.46 (s, 1H)

### (b) 5-Benzyloxymethyl-2-chloro-4-oxo-4,5-dihydroimidazo[4,5-d]pyridazine-1-sulfonic acid dimethylamide

5.3 ml of n-butyl lithium (2.0 M cyclohexane solution) was added to a 150-ml tetrahydrofuran solution of 3.34 g of 5-benzyloxymethyl-4-oxo-4,5-dihydroimidazo[4,5-d]pyridazine-1-sulfonic acid dimethylamide under a nitrogen atmosphere at −78° C., and the mixture was stirred at −78° C. for one hour. Then, 20 ml of a tetrahydrofuran solution of 3.26 g of hexachloroethane was added to this solution. The mixture was allowed to warm to room temperature. 25 ml of a 5% aqueous solution of ammonium chloride was added to the solution, and the mixture was extracted with 50 ml of ethyl acetate. The organic layer washed successively with 25 ml of water and 25 ml of a saturated sodium chloride solution, and then dried over anhydrous magnesium sulfate. The organic liquid was concentrated under reduced pressure. The residue was purified by silica gel column chromatography. Thus, 2.31 g of the title compound was obtained from the fraction eluted with hexane-ethyl acetate (2:3).

¹H-NMR(CDCl₃)

δ 3.12 (s, 6H) 4.77 (s, 2H) 5.70 (s, 2H) 7.30-7.39 (m, 5H) 8.48 (s, 1H)

### (c) t-Butyl 4-(6-benzyloxymethyl-7-oxo-6,7-dihydro-1H-imidazo[4,5-d]pyridazin-2-yl)piperazine-1-carbox ylate

A mixture consisting of 2.31 g of 5-benzyloxymethyl-2-chloro-4-oxo-4,5-dihydroimidazo[4,5-d]pyridazine-1-sulfonic acid dimethylamide and 4.49 g of t-butyl piperazine-1-carboxylate was heated at 150° C. under nitrogen atmosphere for 2.5 hours. The residue was purified by silica gel column chromatography. Thus, 1.94 g of the title compound was obtained from the fraction eluted with ethyl acetate.

¹H-NMR(CDCl₃)

δ 3.54-3.58 (m, 4H) 3.71-3.75 (m, 4H) 4.68 (s, 2H) 5.65 (s, 2H) 7.25-7.35 (m, 5H) 8.21 (s, 1H) 12.58 (br.s, 1H)

### (d) t-Butyl 4-[6-benzyloxymethyl-1-(2-butynyl)-7-oxo-6,7-dihydro-1H-imidazo[4,5-d]pyridazin-2-yl]piperazine-1-carboxylate

0.74 g of potassium carbonate and 0.078 g of 2-butynyl bromide were added to a 20-ml N,N-dimethylformamide solution of 0.216 g of t-butyl 4-(6-benzyloxymethyl-7-oxo-6,7-dihydro-1H-imidazo[4,5-d]pyridazin-2-yl)piperazine-1-carboxylate, and the mixture was stirred at room temperature for 16 hours. Then, 50 ml of ethyl acetate was added to the solution. The organic layer was washed three times with 20 ml of water, and then with 10 ml of a saturated sodium chloride solution. The solution was dried over anhydrous magnesium sulfate, and then concentrated under reduced pressure. The residue was purified by silica gel column chromatography. Thus, 0.139 g of the title compound was obtained from the fraction eluted with hexane-ethyl acetate (3:2).

¹H-NMR(CDCl₃)

δ 1.50 (s, 9H) 1.86 (t, J=2.3 Hz, 3H) 3.38-3.44 (m, 4H) 3.61-3.66 (m, 4H) 4.72 (s, 2H) 5.10 (q, J=2.3 Hz, 2H) 5.65 (s, 2H) 7.25-7.38 (m, 5H) 8.18 (s, 1H)

### (e) 5-Benzyloxymethyl-3-(2-butynyl)-2-(piperazin-1-yl)-3,5-dihydroimidazo[4,5-d]pyridazin-4-one trifluoroacetate

0.0043 g of the title compound was obtained by treating 0.0073 g of t-butyl 4-[6-benzyloxymethyl-1-(2-butynyl)-7-oxo-6,7-dihydro-1H-imidazo[4,5-d]pyridazin-2-yl]pipera zine-1-carboxylate and purifying the product by the same method used in Example 115(i).

¹H-NMR(CD₃OD)

δ 1.83 (t, J=2.3 Hz, 2H) 3.45-3.49 (m, 4H) 3.65-3.69 (m, 4H) 4.69 (s, 2H) 5.15 (q, J=2.3 Hz, 2H) 5.64 (s, 2H) 7.17-7.32 (m, 5H) 8.20 (s, 1H)

MS m/e (ESI) 393.28(MH⁺—CF₃COOH)

### EXAMPLE 117

### 3-(2-Butynyl)-2-(piperazin-1-yl)-3,5-dihydroimidazo[4,5-d]pyridazin-4-one trifluoroacetate

8 ml of a dichloromethane solution of 0.123 g of t-butyl 4-[6-benzyloxymethyl-1-(2-butynyl)-7-oxo-6,7-dihydro-1H-imidazo[4,5-d]pyridazin-2-yl]pipera zine-1-carboxylate was cooled to −78° C. under a nitrogen atmosphere, and 1.9 ml of boron trichloride (1.0 M dichloromethane solution) was added thereto. The mixture was stirred at −78° C. for five hours, and 10 ml of a 1:1 mixed solvent of dichloromethane-methanol was added thereto. The mixture was stirred at −78° C. for two hours, and then allowed to warm to room temperature. The solvent was concentrated under reduced pressure, and 10 ml of methanol was added thereto. Then, the solution was again concentrated under reduced pressure. The residue was dissolved in 3 ml of pyridine, and the mixture was heated under reflux for two hours. 0.3 ml of this solution was concentrated under reduced pressure. The residue was purified by reverse-phase high performance liquid chromatography (using an acetonitrile-water mobile phase (containing 0.1% trifluoroacetic acid)) to give 0.005 g of the title compound.

¹H-NMR(CD₃OD)

δ 1.83 (t, J=2.3 Hz, 3H) 3.45-3.49 (m, 4H) 3.65-3.69 (m, 4H) 5.16 (q, J=2.3 Hz, 2H) 8.21 (s, 1H)

MS m/e (ESI) 273.16 (MH⁺—CF₃COOH)

### EXAMPLE 118

### 2-[7-(2-Butynyl)-1-methyl-6-oxo-8-(piperazin-1-yl)-6,7-dihydro-1H-purin-2-yloxy]benzamide hydrochloride

### (a) t-Butyl 4-[7-(2-butynyl)-2-(2-carbamoylphenoxy)-1-methyl-6-oxo-6,7-dihydro-1H-purin-8-yl]piperazine-1-carboxylate

200 mg of t-butyl 4-[7-(2-butynyl)-2-chloro-1-methyl-6-oxo-6,7-dihydro-1H-purin-8-yl]piperazine-1-carboxylate was dissolved in 2.0 ml of 1-methyl-2-pyrrolidone, and 85 mg of salicylamide and 129 mg of potassium carbonate were added thereto. The mixture was stirred at 100° C. for two hours. After the reaction mixture had been cooled to room temperature, 5.0 ml of water was added thereto. After the mixture had been stirred at room temperature for one hour, the white precipitate was collected by filtration. The resulting white solid washed with water and ether to give of 221 mg of the title compound (89%).

¹H-NMR(DMSO-d6)

δ 1.43 (s, 9H) 1.79 (t, J=2.5 Hz, 3H) 3.23-3.27 (m, 4H) 3.36 (s, 3H) 3.48-3.52 (m, 4H) 4.95 (q, 2.5 Hz, 2H) 6.59 (td, J=8.0, 1.0 Hz, 1H) 6.63 (dd, J=8.0, 1.0 Hz, 1H) 7.14 (ddd, J=8.0, 7.5, 2.0 Hz, 1H) 7.80 (dd, J=7.5, 2.0 Hz, 1H)

MS m/e (ESI) 522(MH⁺)

### (b) 2-[7-(2-Butynyl)-1-methyl-6-oxo-8-(piperazin-1-yl)-6,7-dihydro-1H-purin-2-yloxy]benzamide hydrochloride

210 mg of t-butyl 4-[7-(2-butynyl)-2-(2-carbamoylphenoxy)-1-methyl-6-oxo-6,7-dihydro-1H-purin-8-yl]piperazine-1-carboxylate was combined with 3.5 ml of methanol and 2.1 ml of 4N hydrochloric acid-ethyl acetate solution. After the mixture had been stirred at room temperature for four hours, the reaction solution was concentrated by flushing with nitrogen gas. The resulting residue was washed with ethanol and ethyl acetate to give 177 mg of the title compound (96%).

¹H-NMR(DMSO-d6)

δ 1.82 (t, J=2.3 Hz, 3H) 3.28-3.32 (m, 4H) 3.48 (s, 3H) 3.54-3.58 (m, 4H) 5.04 (q, 2.3 Hz, 2H) 6.96 (br.t, J=7.0 Hz, 1H) 6.99 (br.d, J=8.0 Hz, 1H) 7.46 (ddd, J=8.0, 7.0, 1.5 Hz, 1H) 7.93 (br.d, J=8.0 Hz, 1H)

MS m/e (ESI) 422(MH⁺—HCl)

### EXAMPLE 119

### 3-(2-Butynyl)-5-methyl-2-(piperazin-1-yl)-3,5-dihydroimidazo[4,5-d]pyridazin-4-one

### (a) 5-Methyl-1-trityl-1,5-dihydroimidazo[4,5-d]pyridazin-4-one

78.8 g of 5-methyl-1,5-dihydroimidazo[4,5-d]pyridazin-4-one [CAS No. 76756-58-6] (Shih-Fong Chen and Raymond P. Panzica, Journal of Organic Chemistry 46, p 2467, 1981) was suspended in 2.5 L of dichloromethane at room temperature, and 78.8 of triethylamine was added thereto. 176 g of trityl chloride was added to the mixture, which was then stirred for three hours. 7.5 L of ethyl acetate was added to the mixture. After being washed successively with 3 L of water and 3 L of a saturated sodium chloride solution, the mixture was dried over anhydrous magnesium sulfate and concentrated under reduced pressure. The residue was purified by silica gel column chromatography. Thus, 136.5 g of the title compound was obtained from the fraction eluted with hexane-ethyl acetate (20:80 to 0:100).

¹H-NMR(CDCl₃)

δ 3.79 (s, 3H) 6.92 (s, 1H) 7.07-7.13 (m, 6H) 7.32-7.40 (m, 9H) 7.87 (s, 1H)

### (b) 2-Chloro-5-methyl-1-trityl-1,5-dihydroimidazo[4,5-d]pyridazin-4-one

220 ml of lithium hexamethyldisilazide (1.0 M tetrahydrofuran solution) was added to a 4-L tetrahydrofuran solution of 68.3 g of 5-methyl-1-trityl-1,5-dihydroimidazo[4,5-d]pyridazin-4-one at −75° C. under a nitrogen atmosphere, and the mixture was stirred at −75° C. for one hour. Then, 200 ml of a tetrahydrofuran solution of 82.3 g of hexachloroethane was added to the solution. The mixture was allowed to warm to −20° C. 5 L of 5% aqueous ammonium chloride was added, and the mixture was extracted with 4 L of ethyl acetate. The organic layer washed successively with 5 L of water and 5 L of a saturated sodium chloride solution. The solution was dried over anhydrous magnesium sulfate, and concentrated under reduced pressure. The residue was suspended in 150 ml of t-butyl methyl ether, and then collected by filtration. The solid was washed twice with 100 ml of t-butyl methyl ether to give 69.7 g of the title compound.

¹H-NMR(CDCl₃)

δ 3.78 (s, 3H) 5.81 (s, 1H) 7.25-7.27 (m, 6H) 7.28-7.38 (m, 9H)

### (c) t-Butyl 4-(6-methyl-7-oxo-6,7-dihydro-1H-imidazo[4,5-d]pyridazin-2-yl)piperazine-1-carboxylate

69.7 g of 2-chloro-5-methyl-1-trityl-1,5-dihydroimidazo[4,5-d]pyridazin-4-one was combined with 153.4 g of t-butyl piperazine-1-carboxylate, and the mixture was stirred and heated to 100° C. under a nitrogen atmosphere. When the reaction mixture became easily stirrable, the temperature was raised to 150° C. The mixture was kept at this temperature for one hour. The reaction solution was allowed to cool and then suspended in 250 ml of t-butyl methyl ether. The suspended material was collected by filtration. The solid washed twice with 200 ml of t-butyl methyl ether and three times with 200 ml of water. The solid was again washed twice with 200 ml of t-butyl methyl ether, and dried to give 50.3 g of the title compound.

¹H-NMR(CDCl₃)

δ 1.50 (s, 9H) 3.56-3.62 (m, 4H) 3.73-3.80 (m, 4H) 3.87 (s, 3H) 8.16 (s, 1H) 12.65 (br.s, 1H)

### (d) t-Butyl 4-[1-(2-butynyl)-6-methyl-7-oxo-6,7-dihydro-1H-imidazo[4,5-d]pyridazin-2-yl]piperazine-1-car boxylate

43.9 g of potassium carbonate and 27.8 ml of 2-butynyl bromide were successively added to a 5,5-L N,N-dimethylformamide solution of 88.4 g of t-butyl 4-(6-methyl-7-oxo-6,7-dihydro-1H-imidazo[4,5-d]pyridazin-2-yl)piperazine-1-carboxylate at 15° C. under a nitrogen atmosphere. The reaction solution was stirred at room temperature for 22 hours, and then poured into 10 L of water. The mixture was extracted with 5 L of ethyl acetate. The organic layer was successively washed twice with 5 L of water, and with 5 L of a saturated sodium chloride solution. The aqueous layer was extracted twice with 3 L of ethyl acetate. The organic layers were combined together, and then dried over anhydrous magnesium sulfate. The organic layer was concentrated under reduced pressure. The residue was purified by silica gel column chromatography. Thus, 54.3 g of the title compound was obtained from the fraction eluted with hexane-ethyl acetate (3:2 to 3:7).

¹H-NMR(CDCl₃)

δ 1.52 (s, 9H) 1.83 (t, J=2.3 Hz, 3H) 3.38-3.42 (m, 4H) 3.61-3.64 (m, 4H) 3.85 (s, 3H) 5.09 (q, J=2.3 Hz, 2H) 8.13 (s, 1H)

### (e) 3-(2-Butynyl)-5-methyl-2-(piperazin-1-yl)-3,5-dihydroimidazo[4,5-d]pyridazin-4-one

200 ml of trifluoroacetic acid was added to 200 ml of a dichloromethane solution containing 54.3 g of t-butyl 4-[1-(2-butynyl)-6-methyl-7-oxo-6,7-dihydro-1H-imidazo[4,5-d]pyridazin-2-yl]piperazine-1-car boxylate, and the mixture was stirred at room temperature for one hour. The mixture was concentrated under reduced pressure, and then the residue was dissolved in 500 ml of ethyl acetate. 1 L of 10% aqueous sodium bicarbonate solution was gradually added. Then, 1 L of ethyl acetate and 500 ml of a 5N aqueous sodium hydroxide solution were added to the solution. The organic layer was separated. Then, the aqueous layer was extracted five times with 1 L of dichloromethane. The organic layers were combined together, washed with 500 ml of an aqueous solution of 2N sodium hydroxide, dried over anhydrous magnesium sulfate, and concentrated under reduced pressure. The residue was recrystallized from ethyl acetate to give 30.5 g of the crystalline title compound.

¹H-NMR(CDCl₃)

δ 1.84 (t, J=2.3 Hz, 3H) 3.05-3.09 (m, 4H) 3.38-3.44 (m, 4H) 3.85 (s, 3H) 5.06 (q, J=2.3 Hz, 2H) 8.13 (s, 3H)

### EXAMPLE 119-2

### 3-(2-Butynyl)-5-methyl-2-(piperazin-1-yl)-3,5-dihydroimidazo[4,5-d]pyridazin-4-one toluene-4-sulfonate

98.7 mg of 3-(2-butynyl)-5-methyl-2-(piperazin-1-yl)-3,5-dihydroimidazo[4,5-d]pyridazin-4-one was dissolved in 1 ml of ethanol, and then 1 ml of an ethanol solution of 101 mg of p-toluenesulfonic acid monohydrate was added thereto while the solution was being stirred. The mixture was cooled with ice for two hours while being stirred. The precipitate was collected by filtration, and then dried under reduced pressure at 50° C. for one hour to give 153.2 mg of the title compound.

¹H-NMR (DMSO-d6)

δ 1.79 (t, J=2 Hz, 3H) 2.27 (s, 3H) 3.25-3.35 (m, 4H) 3.50-3.54(m, 4H) 3.70 (s, 3H) 5.13 (d, J=2 Hz, 2H) 7.10 (d, J=8 Hz, 2H) 7.47 (d, J=8 Hz, 2H) 8.25 (s, 1H) 8.79 (br.s, 2H)

Further, 107.95 mg of the title compound was recrystallized from acetone, yielding 84.9 mg of crystalline product.

### EXAMPLE 120

### 2-(3-Aminopiperidin-1-yl)-3-(2-butynyl)-5-methyl-3,5-dihydroimidazo[4,5-d]pyridazin-4-one trifluoroacetate

### (a) 9H-fluoren-9-ylmethyl 3-t-butoxycarbonylaminopiperidine-1-carboxylate

1.84 g of diisopropylethylamine and 4.71 g of diphenylphosphorylazide were added to 10 ml of a t-butanol solution of 5.01 g of 9H-fluoren-9-ylmethyl 3-carboxypiperidine-1-carboxylate, and the mixture was heated at 60° C. under a nitrogen atmosphere for 18 hours. The reaction solution was cooled, and 150 ml of ethyl acetate was added thereto. The organic layer was washed successively with 100 ml of 5% aqueous sulfuric acid, 100 ml of 5% aqueous sodium bicarbonate solution, 100 ml of water, and 100 ml of a saturated sodium chloride solution, and then dried over anhydrous magnesium sulfate. The organic layer was concentrated under reduced pressure. The residue was purified by silica gel column chromatography. Thus, 1.88 g of the title compound was obtained from the fraction eluted with hexane-ethyl acetate (4:1).

¹H-NMR(CDCl₃)

δ 1.45 (s, 9H) 1.45-1.72 (m, 3H) 1.82-1.87 (br.s, 1H) 3.09-3.30 (br.s, 2H) 3.58 (br.s, 2H) 3.82-3.98 (br.s, 1H) 4.24 (t, J=7.2 Hz, 1H) 4.27-4.48 (br.s, 2H) 4.52-4.59 (br.s, 1H) 7.32 (dd, J=10.3, 10.0 Hz, 2H) 7.39 (t, J=10.0 Hz, 2H) 7.59 (d, J=10.0 Hz, 2H) 7.75 (d, J=10.3 Hz, 2H)

### (b) t-Butyl piperidin-3-ylcarbamate

25 ml of diethylamine was added to 250 ml of an ethanol solution of 1.88 g of 9H-fluoren-9-ylmethyl 3-t-butoxycarbonylaminopiperidine-1-carboxylate, and the mixture was stirred at room temperature for 18 hours. After the solution had been concentrated under reduced pressure, the residue was dissolved in a mixture consisting of 150 ml of toluene and 100 ml of 10% aqueous citric acid solution. The aqueous layer was made alkaline with a 5N aqueous sodium hydroxide solution, and then extracted twice with 100 ml of dichloromethane. The organic layers were combined together, dried over anhydrous magnesium sulfate, and concentrated under reduced pressure to give 0.79 g of the title compound.

¹H-NMR(CDCl₃)

δ 1.45 (s, 9H) 1.41-1.53 (m, 2H) 1.65-1.72 (m, 1H) 1.79-1.86 (m, 1H) 2.48-2.56 (m, 1H) 2.64-2.70 (m, 1H) 2.78-2.86 (m, 1H) 3.06 (dd, J=12.0, 4.0 Hz, 1H) 3.48-3.62 (br.s, 1H) 4.71-4.88 (br.s, 1H)

### (c) 2-(3-Aminopiperidin-1-yl)-3-(2-butynyl)-5-methyl-3,5-dihydroimidazo[4,5-d]pyridazin-4-one trifluoroacetate

0.020 g of 2-chloro-5-methyl-1-trityl-1,5-dihydroimidazo[4,5-d]pyridazine-4-one and 0.040 g of t-butyl piperidin-3-ylcarbamate were combined together, and the mixture was heated under a nitrogen atmosphere at 150° C. for one hour. The reaction mixture was purified by silica gel column chromatography. Thus, 0.016 g of t-butyl [1-(6-methyl-7-oxo-6,7-dihydro-1H-imidazo[4,5-d]pyridazin-2-yl)piperidin-3-yl]carbamate was obtained from the fraction eluted with ethyl acetate. 0.0080 g of this compound was dissolved in 0.6 ml of N,N-dimethylformamide, and then 0.0038 g of potassium carbonate and 0.003 ml of 2-butynyl bromide were added thereto. The mixture was stirred at room temperature for 18 hours. The reaction mixture was partitioned between 1 ml of ethyl acetate and 1 ml of water, and the organic layer was concentrated. The residue was dissolved in 0.5 ml of dichloromethane, and then 0.5 ml of trifluoroacetic acid was added thereto. After one hour, the reaction solution was concentrated. The residue was purified by reverse-phase high performance liquid chromatography (using an acetonitrile-water mobile phase (containing 0.1% trifluoroacetic acid)) to give 0.0046 g of the title compound.

¹H-NMR(CDCl₃)

δ 1.74-1.80 (br.s, 1H) 1.82 (br.s, 3H) 1.96-2.19 (br.m, 3H) 3.43-3.79 (br.m, 5H) 3.86 (s, 3H) 5.05 (br.d, J=16.0 Hz, 1H) 5.23 (br.d, J=16.0 Hz, 1H) 8.15 (s, 1H)

### EXAMPLE 122

### 2-[7-(2-Butynyl)-1-methyl-6-oxo-8-(piperazin-1-yl)-6,7-dihydro-1H-purin-2-yloxy]benzamide

53.0 g of t-butyl 4-[7-(2-butynyl)-2-(2-carbamoylphenoxy)-1-methyl-6-oxo-6,7-dihydro-1H-purin-8-yl]piperazine-1-carboxylate was dissolved in 160 ml of trifluoroacetic acid, and the mixture was stirred at room temperature for one hour. 1250 ml of a 2 M aqueous sodium hydroxide solution was added dropwise to the reaction solution, and the mixture was stirred at room temperature for one hour and 50 minutes. The resulting white precipitate was collected by filtration. The white solid washed with water and then with ethanol, and dried at 60° C. overnight to give 42.8 g of the title compound.

¹H-NMR(DMSO-d6)

δ 1.78 (t, J=2.4 Hz, 3H) 2.82-2.86 (m, 4H) 3.18-3.22 (m, 4H) 3.36 (s, 3H) 4.91 (q, 2.4 Hz, 2H) 6.58 (td, J=8.4, 1.2 Hz, 1H) 6.63 (dd, J=8.0, 0.8 Hz, 1H) 7.14 (ddd, J=8.0, 7.2, 2.0 Hz, 1H) 7.80 (dd, J=7.6, 2.0 Hz, 1H)

MS m/e (ESI) 422(MH⁺)

### EXAMPLE 229

### 7-(2-Butynyl)-1-(2-cyanobenzyl)-6-oxo-8-(piperazin-1-yl)-6,7-dihydro-1H-purine-2-carbonitrile hydrochloride

### (a) t-Butyl 4-[7-(2-butynyl)-2-cyano-1-(2-cyanobenzyl)-6-oxo-6,7-dihydro-1H-purin-8-yl]piperazine-1-carb oxylate

A mixture consisting of 8 mg of t-butyl 4-[7-(2-butynyl)-2-chloro-1-(2-cyanobenzyl)-6-oxo-6,7-dihydro-1H-purin-8-yl]piperazine-1-car boxylate obtained in Example 96(a), 10 mg of sodium cyamide and 0.3 ml of N,N-dimethylformamide was stirred at room temperature for four hours. The reaction mixture was extracted with ethyl acetate-water, and the organic layer washed with water and then with saturated brine. The organic layer was concentrated. The residue was purified by thin layer chromatography (50% ethyl acetate/hexane) to give 6.1 mg of the title compound.

¹H-NMR(CDCl₃)

δ 1.50 (s, 9H) 1.83 (s, 3H) 3.50 (s, 4H) 3.58-3.64 (m, 4H) 4.99 (s, 2H) 5.74 (s, 2H) 7.02 (d, J=8 Hz, 1H) 7.44 (t, J=8 Hz, 1H) 7.55 (t, J=8 Hz, 1H) 7.74 (d, J=8 Hz, 1H)

### 7-(2-Butynyl)-1-(2-cyanobenzyl)-6-oxo-8-(piperazin-1-yl)-6,7-dihydro-1H-purine-2-carbonitrile hydrochloride

A mixture consisting of 6.1 mg of t-butyl 4-[7-(2-butynyl)-2-cyano-1-(2-cyanobenzyl)-6-oxo-6,7-dihydro-1H-purin-8-yl]piperazine-1-carb oxylate and 0.2 ml of trifluoroacetic acid was stirred at room temperature for 20 minutes. The reaction solution was concentrated, and the residue was purified by reverse-phase column chromatography using a 20% to 60% methanol/water (0.1% concentrated hydrochloric acid) solvent to give 5.0 mg of the title compound.

¹H-NMR(DMSO-d6)

δ 1.80 (s, 3H) 3.30 (s, 4H) 3.60-3.70 (m, 4H) 5.09 (s, 2H) 5.60 (s, 2H) 7.27 (d, J=8 Hz, 1H) 7.54 (t, J=8 Hz, 1H) 7.68 (t, J=8 Hz, 1H) 7.94 (d, J=8 Hz, 1H) 9.36 (br.s, 2H)

### EXAMPLE 230

### 3-[7-(2-Butynyl)-1-(2-cyanobenzyl)-6-oxo-8-(piperazin-1-yl)-6,7-dihydro-1H-purin-2-yloxy]pyridine-2-carboxylic amide trifluoroacetate

7 mg of t-butyl 4-[7-(2-butynyl)-2-chloro-1-(2-cyanobenzyl)-6-oxo-6,7-dihydro-1H-purin-8-yl]piperazine-1-carboxylate was dissolved in 0.2 ml of 1-methyl-2-pyrrolidone, and then 8 mg of 3-hydroxypyridine-2-carboxylic amide and 8 mg of potassium carbonate were added thereto. The mixture was stirred at 100° C. for two hours. 1N hydrochloric acid was added to the reaction mixture, and the mixture was extracted with ethyl acetate. The organic layer was concentrated, and the residue was dissolved in trifluoroacetic acid. The solution was concentrated, and the residue was purified by reverse-phase high performance liquid chromatography (using an acetonitrile-water mobile phase (containing 0.1% trifluoroacetic acid)) to give 2.93 mg of the title compound.

MS m/e (ESI) 524(MH⁺—CF₃COOH)

### EXAMPLE 234

### 2-[7-(2-Butynyl)-1-(2-cyanobenzyl)-6-oxo-8-(piperazin-1-yl)-6,7-dihydro-1H-purin-2-yloxy]benzamide trifluoroacetate

3.74 mg of the title compound was obtained by using salicylamide, instead of 3-hydroxypyridine-2-carboxylic amide, according to the method described in Example 230.

MS m/e (ESI) 523(MH⁺—CF₃COOH)

### EXAMPLE 242

### 8-(3-amino piperidin-1-yl)-7-(2-butynyl)-1-(2-cyanobenzyl)-6-oxo-6,7-dihydro-1H-purine-2-carbonitrile hydrochloride

### (a) Benzyl 3-t-butoxycarbonylaminopiperidine-1-carboxylate

88 g of benzyl chloroformate (30% toluene solution) was added dropwise to a mixture consisting of 24.3 g of ethyl piperidine-3-carboxylate, 26 ml of triethylamine and 300 ml of ethyl acetate over 30 minutes while the mixture was being cooled with ice. The reaction mixture was filtered to remove insoluble material. The filtrate was again filtered through a small amount of silica gel. The filtrate was concentrated.

200 ml of ethanol and 40 ml of a 5 M aqueous sodium hydroxide solution were added to the residue. The mixture was stirred at room temperature overnight. The reaction solution was concentrated, and 200 ml of water was added to the residue. The mixture was extracted with t-butyl methyl ether. 5 M aqueous hydrochloric acid was added to the aqueous layer, and the mixture was extracted with ethyl acetate. The organic layer washed with water and then with saturated brine. The organic layer was dried over anhydrous magnesium sulfate, and then concentrated to give an oily residue (30.9 g).

A mixture consisting of 30 g of this residue, 24.5 ml of diphenyl phosphoryl azide, 15.9 ml of triethylamine and 250 ml of t-butanol was stirred at room temperature for 1.5 hours. The mixture was further stirred in an oil bath at 100° C. for 20 hours. The reaction solution was concentrated, and the residue was extracted with ethyl acetate-water. The organic layer was washed with dilute aqueous sodium bicarbonate solution and then with saturated brine. The organic layer was dried over anhydrous magnesium sulfate, and then concentrated. The residue was purified by silica gel column chromatography using 10% to 20% ethyl acetate/hexane, followed by recrystallization from ethyl acetate-hexane to give 21.4 g of the title compound.

¹H-NMR(CDCl₃)

δ 1.43 (s, 9H) 1.48-1.92 (m, 4H) 3.20-3.80 (m, 5H) 4.58 (br.s, 1H) 5.13 (s, 2H) 7.26-7.40(m, 5H)

### (b) t-Butyl piperidin-3-ylcarbamate

A mixture consisting of 10 g of benzyl 3-t-butoxycarbonylaminopiperidine-1-carboxylate, 500 mg of 10% palladium carbon and 100 ml of ethanol was stirred at room temperature under a hydrogen atmosphere overnight. The catalyst was removed by filtration. The filtrate was concentrated and dried to give 6.0 g of the title compound.

¹H-NMR(CDCl₃)

δ1.44 (s, 9H) 1.47-1.80 (m, 4H) 2.45-2.60 (m, 1H) 2.60-2.75 (m, 1H) 2.75-2.90 (m, 1H) 3.05 (dd, J=3 Hz, 12 Hz, 1H) 3.57 (br.s, 1H) 4.83 (br.s, 1H)

### (c) t-Butyl[1-[7-(2-butynyl)-2,6-dichloro-7H-purin-8-yl]piperidin-3-yl]carbamate

A mixture consisting of 1.25 g of 7-(2-butynyl)-2,6,8-trichloro-7H-purine, 1.0 g of t-butyl piperidin-3-ylcarbamate and 10 ml of acetonitrile was stirred at room temperature for 10 minutes. 0.63 ml of triethylamine was added dropwise over 10 minutes, and then the mixture was continuously stirred at room temperature for 30 minutes. The reaction solution was partitioned between ethyl acetate and water, and the organic layer washed with saturated brine. The organic layer was dried over anhydrous magnesium sulfate, and then concentrated. The residue was crystallized with t-butyl methyl ether-hexane to give 1.79 g of the title compound.

¹H-NMR(CDCl₃)

δ 1.43 (s, 9H) 1.60-2.02 (m, 4H) 1.83 (t, J=2 Hz, 3H) 3.32-3.41 (m, 1H) 3.42-3.52 (m, 1H) 3.67-3.76 (m, 1H) 3.80-3.91 (m, 1H) 4.76-4.90 (m, 3H)

### (d) t-Butyl [1-[7-(2-butynyl)-2-chloro-6-oxo-6,7-dihydro-1H-purin-8-yl]piperidin-3-yl]carbamate

A mixture consisting of 1.79 g of t-butyl [1-[7-(2-butynyl)-2,6-dichloro-7H-purin-8-yl]piperidin-3-yl]carbamate, 1.0 g of sodium acetate and 18 ml of dimethyl sulfoxide was stirred in an oil bath at 120° C. for three hours. The mixture was removed from the oil bath, and 18 ml of water was added to the reaction solution. The mixture was cooled to room temperature. The crystals were collected by filtration, and washed with water and then with t-butyl methyl ether. The crystals were then dried to give 1.59 g of the title compound.

¹H-NMR(DMSO-d6)

δ 1.39 (s, 9H) 1.34-1.88 (m, 4H) 1.78 (s, 3H) 2.81 (t, J=11 Hz, 1H) 2.95 (t, J=11 Hz, 1H) 3.48-3.60 (m, 2H) 3.64 (d, J=6 Hz, 1H) 4.90 (s, 2H) 6.94 (d, J=8 Hz, 1H)

### (e) t-Butyl [1-[7-(2-butynyl)-2-chloro-1-(2-cyanobenzyl)-6-oxo-6,7-dihydro-1H-purin-8-yl]piperidin-3-yl]c arbamate

A mixture consisting of 100 mg of t-butyl [1-[7-(2-butynyl)-2-chloro-6-oxo-6,7-dihydro-1H-purin-8-yl]piperidin-3-yl]carbamate, 66 mg of anhydrous potassium carbonate, 70 mg of 2-cyanobenzyl bromide and 1 ml of N,N-dimethylformamide was stirred at room temperature for five hours. The reaction solution was partitioned between ethyl acetate and water, and the organic layer washed with water and then with saturated brine. The organic layer was dried over anhydrous magnesium sulfate, and then concentrated. The residue was purified by silica gel column chromatography using 50% ethyl acetate/hexane to give 44.7 mg of the title compound.

¹H-NMR(CDCl₃)

δ 1.44 (s, 9H) 1.59-1.81 (m, 2H) 1.83 (t, J=2 Hz, 3H) 1.86-1.94 (m, 2H) 3.20-3.50 (m, 3H) 3.66 (d, J=7 Hz, 1H) 3.86 (br.s, 1H) 4.88-5.06 (m, 3H) 5.72 (s, 2H) 7.06 (d, J=8 Hz, 1H) 7.38 (t, J=8 Hz, 1H) 7.51 (t, J=8 Hz, 1H) 7.70 (d, J=8 Hz, 1H)

### (f) t-Butyl [1-[7-(2-butynyl)-2-cyano-1-(2-cyanobenzyl)-6-oxo-6,7-dihydro-1-purin-8-yl]piperidin-3-yl]carbamate

A mixture consisting of 15 mg of t-butyl[1-[7-(2-butynyl)-2-chloro-1-(2-cyanobenzyl)-6-oxo-6,7-dihydro-1H-purin-8-yl]piperidin-3-yl]carbamate, 20 mg of sodium cyamide and 0.2 ml of N,N-dimethylformamide was stirred at room temperature for three hours. The reaction solution was partitioned between ethyl acetate and water, and the organic layer washed with water and then with saturated brine. Then, the organic layer was concentrated, and the residue was purified by thin layer chromatography using 50% ethyl acetate/hexane solvent (developed three times) to give 10.3 mg of the title compound.

¹H-NMR(CDCl₃)

δ 1.44 (s, 9H) 1.52-1.98 (m, 4H) 1.81 (t, J=2 Hz 3H) 3.24 (dd, J=7 Hz, 12 Hz, 1H) 3.30-3.40 (m, 1H) 3.46-3.56 (m, 1H), 3.72 (d, J=12 Hz, 1H) 3.86 (br.s, 1H) 4.86-5.10 (m, 3H) 5.73 (s, 2H) 7.00 (d, J=8 Hz, 1H) 7.42 (t, J=8 Hz, 1H) 7.54 (dt, J=2 Hz, 8 Hz, 1H) 7.73 (dd, J=2 Hz, 8 Hz, 1H)

### (g) 8-(3-Aminopiperidin-1-yl)-7-(2-butynyl)-1-(2-cyanobenzyl)-6-oxo-6,7-dihydro-1H-purine-2-carbonitrile hydrochloride

A mixture consisting of 10.3 mg of t-butyl [1-[7-(2-butynyl)-2-cyano-1-(2-cyanobenzyl)-6-oxo-6,7-dihydro-1H-purin-8-yl]piperidin-3-yl]carbamate and 0.2 ml of trifluoroacetic acid was stirred for 20 minutes. The reaction solution was concentrated, and the residue was purified by reverse-phase column chromatography using 20% to 80% methanol/water (0.1% concentrated hydrochloric acid) solvent to give 8.0 mg of the title compound.

¹H-NMR(DMSO-d6)

δ 1.60-1.74 (m, 2H) 1.79 (t, J=2 Hz, 3H) 1.88-2.03 (m, 2H) 3.14-3.28 (m, 2H) 3.42 (br.s, 1H) 3.52-3.82 (m, 2H) 4.98-5.12 (m, 2H) 5.58 (s, 2H) 7.26 (d, J=8 Hz, 1H) 7.53 (t, J=8 Hz, 1H) 7.66 (t, J=8 Hz, 1H) 7.93 (d, J=8 Hz, 1H) 8.16 (br.s, 3H)

### EXAMPLE 248

### 2-[8-(3-Aminopiperidin-1-yl)-7-(2-butynyl)-1-methyl-6-oxo-6,7-dihydro-1H-purin-2-yloxy]benz amide trifluoroacetic acid salt

### (a) t-Butyl [1-[7-(2-butynyl)-2-chloro-1-methyl-6-oxo-6,7-dihydro-1H-purin-8-yl]piperidin-3-yl]carbamate

700 mg of t-butyl [1-[7-(2-butynyl)-2-chloro-6-oxo-6,7-dihydro-1H-purin-8-yl]piperidin-3-yl]carbamate was dissolved in 7.0 ml of dimethyl sulfoxide, and then 114 μl of methyl iodide and 299 mg of potassium carbonate were added thereto. The mixture was stirred at room temperature for 30 minutes, and 40 ml of water was added to the reaction solution. The mixture was stirred at room temperature for 30 minutes, and the white precipitate was collected by filtration. The resulting white solid washed with water and then with hexane to give 540 mg of the title compound.

¹H-NMR(CDCl3)

δ 1.44 (s, 9H) 1.72-1.94 (m, 4H) 1.81 (t, J=2.4 Hz, 3H) 3.16-3.92 (m, 5H) 3.72 (s, 3H) 4.91 (dd, J=17.6, 2.4 Hz, 1H) 5.01 (d, J=17.6 Hz, 1H)

### (b) 2-[8-(3-Aminopiperidin-1-yl)-7-(2-butynyl)-1-methyl-6-oxo-6,7-dihydro-1H-purin-2-yloxy]benzamide trifluoroacetate

10 mg of t-butyl [1-[7-(2-butynyl)-2-chloro-1-methyl-6-oxo-6,7-dihydro-1H-purin-8-yl]piperidin-3-yl]carbamate was dissolved in 0.3 ml of 1-methyl-2-pyrrolidone, and then 10 mg of salicylamide and 10 mg of potassium carbonate were added thereto. The mixture was stirred at 100° C. for two hours. 1N hydrochloric acid was added to the reaction solution, and the mixture was extracted with ethyl acetate. The organic layer was concentrated, and the residue was dissolved in trifluoroacetic acid. The solution was concentrated, and the residue was purified by reverse-phase high performance liquid chromatography (using an acetonitrile-water mobile phase (containing 0.1% trifluoroacetic acid)) to give 5.54 mg of the title compound.

MS m/e (ESI) 436(MH⁺—CF₃COOH)

### EXAMPLE 258

### 3-(2-Butynyl)-2-(piperazin-1-yl)-5-(2-propynyl)-3,5-dihydroimidazo[4,5-d]pyridazin-4-one trifluoroacetate

### (a) t-Butyl 4-[1-(2-butynyl)-7-oxo-6,7-dihydro-1H-imidazo[4,5-d]pyridazin-2-yl]piperazine-1-carboxylate

0.299 g of triethylamine, 0.023 g of 4-dimethylaminopyridine and 0.645 g of di-t-butyl dicarbonate were added to 20 ml of an N,N-dimethylformamide solution of 0.448 g of 3-(2-butynyl)-2-(piperazin-1-yl)-3,5-dihydroimidazo[4,5-d]pyridazin-4-one trifluoroacetate at room temperature, and the mixture was stirred for five hours. Then, 2 ml of a 5N aqueous sodium hydroxide solution was added to this solution, and the mixture was stirred for one hour. The reaction solution was poured into a mixture of 200 ml of ethyl acetate and 100 ml of a saturated aqueous ammonium chloride solution. The organic layer washed twice with 100 ml of water and then with 100 ml of a saturated sodium chloride solution. The organic liquid was dried over magnesium sulfate, and concentrated under reduced pressure. The residue was purified by silica gel column chromatography. Thus, 0.298 g of the title compound was obtained from the fraction eluted with ethyl acetate.

¹H-NMR(CDCl₃)

δ 1.50 (s, 9H) 1.84 (t, J=2.3 Hz, 3H) 3.41 (m, 4H) 3.63 (m, 4H) 5.06 (q, J=2.3 Hz, 2H) 8.17 (s, 1H) 9.92 (br.s, 1H)

### (b) 3-(2-Butynyl)-2-(piperazin-1-yl)-5-(2-propynyl)-3,5-dihydroimidazo[4,5-d]pyridazin-4-one trifluoroacetate

0.005 g of potassium carbonate and 0.003 ml of 3-bromo-1-propyne were added to 0.5 ml of an N,N-dimethylformamide solution of 0.010 g of t-butyl 4-[1-(2-butynyl)-7-oxo-6,7-dihydro-1H-imidazo[4,5-d]pyridazin-2-yl]piperazine-1-carboxylate, and the mixture was stirred at room temperature for 10 hours. 1 ml of ethyl acetate and 1 ml of water were added to the reaction solution, and the layers were separated. The organic layer was concentrated, and the resulting residue was dissolved in a mixture consisting of 0.5 ml of dichloromethane and 0.5 ml of trifluoroacetic acid. The mixture was stirred for one hour, and then concentrated. The residue was purified by reverse-phase high performance liquid chromatography (using an acetonitrile-water mobile phase (containing 0.1% trifluoroacetic acid)) to give 0.011 g of the title compound.

MS m/e (ESI) 311.29(MH⁺—CF₃COOH)

### EXAMPLE 266

### 3-(2-Butynyl)-5-[2-(3-methoxyphenyl)-2-oxoethyl]-2-(piperazin-1-yl)-3,5-dihydroimidazo[4,5-d]pyridazin-4-one trifluoroacetate

The title compound was obtained by using t-butyl 4-[1-(2-butynyl)-7-oxo-6,7-dihydro-1H-imidazo[4,5-d]pyridazin-2-yl]piperazine-1-carboxylate and 2-bromo-3′-methoxy acetophenone according to the method described in Example 258(b).

MS m/e (ESI) 421.33(MH⁺—CF₃COOH)

### EXAMPLE 267

### 2-[3-(2-Butynyl)-4-oxo-2-(piperazin-1-yl)-3,4-dihydroimidazo[4,5-d]pyridazin-5-ylmethyl]benzonitrile trifluoroacetate

The title compound was obtained by using t-butyl 4-[1-(2-butynyl)-7-oxo-6,7-dihydro-1H-imidazo[4,5-d]pyridazin-2-yl]piperazine-1-carboxylate and 2-bromomethylbenzonitrile according to the method described in Example 258(b).

¹H-NMR(CD₃OD)

δ 1.81 (t, J=2.5 Hz, 3H) 3.45-3.49 (m, 4H) 3.66-3.70 (m, 4H) 5.15 (q, J=2.5 Hz, 2H) 5.62 (s, 2H) 7.34 (dd, J=7.6, 1.5 Hz, 1H) 7.45 (td, J=7.6, 1.5 Hz, 1H) 7.59 (td, J=7.6, 1.7 Hz, 1H) 7.75 (dd, J=7.6, 1.7 Hz, 1H) 8.25 (s, 1H)

MS m/e (ESI) 388.32(MH⁺—CF₃COOH)

### EXAMPLE 297

### 2-[3-(2-Butynyl)-4-oxo-2-(piperazin-1-yl)-3,4-dihydroimidazo[4,5-d]pyridazin-5-ylmethyl]-3-fluorobenzonitrile trifluoroacetate

The title compound was obtained by using t-butyl 4-[1-(2-butynyl)-7-oxo-6,7-dihydro-1H-imidazo[4,5-d]pyridazin-2-yl]piperazine-1-carboxylate and 2-bromomethyl-3-fluorobenzonitrile according to the method described in Example 258(b).

MS m/e (ESI) 406.25(MH⁺—CF₃COOH)

### EXAMPLE 308

### 3-Benzyl-2-(piperazin-1-yl)-3,5-dihydroimidazo[4,5-d]pyridazin-4-one trifluoroacetate

### (a) t-Butyl 4-(1-benzyl-6-benzyloxymethyl-7-oxo-6,7-dihydro-1H-imidazo[4,5-d]pyridazin-2-yl)piperazine-1-carboxylate

The title compound was obtained by using t-butyl 4-(6-benzyloxymethyl-7-oxo-6,7-dihydro-1H-imidazo[4,5-d]pyridazin-2-yl)piperazine-1-carboxylate and benzyl bromide according to the method described in Example 116(d).

¹H-NMR(CDCl₃)

δ 1.48 (s, 9H) 3.13-3.18 (m, 4H) 3.50-3.54 (m, 4H) 4.72 (s, 2H) 5.61 (s, 2H) 5.65 (s, 2H) 7.20-7.35(m, 10H) 8.22 (s, 1H)

### (b) 3-Benzyl-2-(piperazin-1-yl)-3,5-dihydroimidazo[4,5-d]pyridazin-4-one trifluoroacetate

The title compound was obtained by treating t-butyl 4-(1-benzyl-6-benzyloxymethyl-7-oxo-6,7-dihydro-1H-imidazo[4,5-d]pyridazin-2-yl)piperazine-1-carboxylate according to the method described in Example 117.

¹H-NMR(CD₃OD)

δ 3.31-3.37 (m, 4H) 3.40-3.46 (m, 4H) 5.68 (s, 2H) 7.22-7.36(m, 5H) 8.25 (s, 1H)

MS m/e (ESI) 311.24(MH⁺—CF₃COOH)

### EXAMPLE 309

### 3-Benzyl-5-methyl-2-(piperazin-1-yl)-3,5-dihydroimidazo[4,5-d]pyridazin-4-one trifluoroacetate

### (a) t-Butyl 4-(1-benzyl-7-oxo-6,7-dihydro-1H-imidazo[4,5-d]pyridazin-2-yl)piperazine-1-carboxylate

The title compound was obtained by using 3-benzyl-2-(piperazin-1-yl)-3,5-dihydroimidazo[4,5-d]pyridazin-4-one trifluoroacetate according to the method described in Example 258(a).

¹H-NMR(CDCl₃)

δ 1.47 (s, 9H) 3.12-3.16 (m, 4H) 3.47-3.52 (m, 4H) 5.58 (s, 2H) 7.20-7.34(m, 5H) 8.20 (s, 1H) 10.04 (br.s, 1H)

### (b) 3-Benzyl-5-methyl-2-(piperazin-1-yl)-3,5-dihydroimidazo[4,5-d]pyridazin-4-one trifluoroacetate

The title compound was obtained by using t-butyl 4-(1-benzyl-7-oxo-6,7-dihydro-1H-imidazo[4,5-d]pyridazin-2-yl)piperazine-1-carboxylate and methyl iodide according to the method described in Example 258(b).

¹H-NMR(CD₃OD)

δ 3.29-3.35 (m, 4H) 3.36-3.41 (m, 4H) 3.83 (s, 3H) 5.68 (s, 2H) 7.21-7.34(m, 5H) 8.20 (s, 1H)

MS m/e (ESI) 325.01(MH⁺—CF₃COOH)

### EXAMPLE 311

### 3-Benzyl-5-(2-phenylethyl)-2-(piperazin-1-yl)-3,5-dihydroimidazo[4,5-d]pyridazin-4-one trifluoroacetate

The title compound was obtained by using t-butyl 4-[1-benzyl-7-oxo-6,7-dihydro-1H-imidazo[4,5-d]pyridazin-2-yl]piperazine-1-carboxylate and (2-bromoethyl)benzene according to the method described in Example 258(b).

¹H-NMR(CDCl₃)

δ 3.11 (t, J=8.1 Hz, 2H) 3.24-3.29 (m, 4H) 3.37-3.42 (m, 4H) 4.46 (t, J=8.1 Hz, 2H) 5.58 (s, 2H) 7.09-7.34 (m, 10H) 8.20 (s, 1H)

MS m/e (ESI) 415.54(MH⁺—CF₃COOH)

### EXAMPLE 332

### 1-(2-Butynyl)-6-methyl-7-oxo-2-(piperazin-1-yl)-6,7-dihydroimidazo[4,5-d]pyridazine-4-carboxamide trifluoroacetate

### (a) t-Butyl 4-[1-(2-butynyl)-4-(cyano-hydroxymethyl)-5-methoxycarbonyl-1H-imidazol-2-yl]piperazine-1-carboxylate

0.200 g of sodium cyamide and 0.010 ml of acetic acid were added to a 15 ml acetonitrile solution of t-butyl 4-[1-(2-butynyl)-5-methoxycarbonyl-4-formyl-1H-imidazol-2-yl]piperazine-1-carboxylate, and the mixture was stirred at room temperature for 16 hours. 100 ml of ethyl acetate was added to the solution, and the mixture washed twice with 50 ml of water and then with 50 ml of a saturated sodium chloride solution. The organic layer was dried over magnesium sulfate, and the solvent was concentrated under reduced pressure. The residue was purified by silica gel column chromatography. Thus, 0.274 g of the title compound was obtained from the fraction eluted with ethyl acetate-hexane (2:3).

¹H-NMR(CDCl₃)

δ 1.49 (s, 9H) 1.83 (t, J=2.5 Hz, 3H) 3.19-3.23 (m, 4H) 3.56-3.60 (m, 4H) 3.95 (s, 3H) 4.68 (d, J=9.0 Hz, 1H) 4.82 (q, J=2.5 Hz, 2H) 5.72 (d, J=9.0 Hz, 1H)

### (b) t-Butyl 4-[1-(2-butynyl)-4-(carbamoyl-hydroxymethyl)-5-methoxycarbonyl-1H-imidazol-2-yl]piperazine-1-carboxylate

3.2 ml of 30% aqueous hydrogen peroxide and 3.2 ml of 28% aqueous ammonia solution were added to an 8 ml methanol solution of 0.274 g of t-butyl 4-[1-(2-butynyl)-4-(cyano-hydroxymethyl)-5-methoxycarbonyl-1H-imidazol-2-yl]piperazine-1-carboxylate at 5° C., and the mixture was stirred for 15 hours. 100 ml of a saturated sodium hydrogen sulfite solution was added to the solution, and the mixture was extracted twice with 100 ml of ethyl acetate. The organic layers were combined together. The combined organic layers were dried over magnesium sulfate, and concentrated under reduced pressure. The residue was purified by silica gel column chromatography. Thus, 0.039 g of the title compound was obtained from the fraction eluted with methanol-ethyl acetate (1:9).

¹H-NMR(CDCl₃)

δ 1.48 (s, 9H) 1.83 (t, J=2.5 Hz, 3H) 3.13-3.25 (m, 4H) 3.54-3.57 (m, 4H) 3.91 (s, 3H) 4.33-4.37 (br.s, 1H) 4.77 (q, J=2.5 Hz, 2H) 5.54 (s, 1H) 5.63 (s, 1H) 6.82 (s, 1H)

### (c) t-Butyl 4-[4-aminooxalyl-1-(2-butynyl)-5-methoxycarbonyl-1H-imidazol-2-yl]piperazine-1-carboxylate

0.051 ml of triethylamine and a 1 ml dimethyl sulfoxide solution of 0.058 g of sulfur trioxide pyridine were added to a 2 ml dichloromethane solution of 0.038 g of t-butyl 4-[1-(2-butynyl)-4-(carbamoyl-hydroxymethyl)-5-methoxycarbonyl-1H-imidazol-2-yl]piperazine-1-carboxylate at 0° C., and the mixture was stirred at room temperature for 15 hours. Then, 0.102 ml of triethylamine and a 1 ml dimethyl sulfoxide solution of 0.116 g of sulfur trioxide pyridine were added, and the mixture was stirred at room temperature for eight hours. 50 ml of ethyl acetate was added to the solution, and the organic layer washed successively with 20 ml of an aqueous solution of 1% sulfuric acid, 20 ml of a saturated sodium bicarbonate solution, and 20 ml of a saturated sodium chloride solution. The organic layer was dried over magnesium sulfate, and concentrated under reduced pressure. The residue was purified by silica gel column chromatography. Thus, 0.021 g of the title compound was obtained from the fraction eluted with ethyl acetate-hexane (2:1).

¹H-NMR(CDCl₃)

δ 1.48 (s, 9H) 1.82 (t, J=2.5 Hz, 3H) 3.19-3.23 (m, 4H) 3.56-3.59 (m, 4H) 3.84 (s, 3H) 4.84 (q, J=2.5 Hz, 2H) 5.62 (br.s, 1H) 7.02 (br.s, 1H)

### (d) t-Butyl 4-[1-(2-butynyl)-4-carbamoyl-6-methyl-7-oxo-6,7-dihydro-1H-dihydroimidazo[4,5-d]pyridazin-2-yl]piperazine-1-carboxylate

The title compound was obtained by using t-butyl 4-[4-aminooxalyl-1-(2-butynyl)-5-methoxycarbonyl-1H-imidazol-2-yl]piperazine-1-carboxylate according to the method described in Example 115(h).

¹H-NMR(CDCl₃)

δ 1.50 (s, 9H) 1.84 (t, J=2.3 Hz, 3H) 3.46-3.50 (m, 4H) 3.63-3.66 (m, 4H) 3.99 (s, 3H) 5.12 (q, J=2.3 Hz, 2H) 6.16 (s, 1H) 8.85 (s, 1H)

### (e) 1-(2-Butynyl)-6-methyl-7-oxo-2-(piperazin-1-yl)-6,7-dihydroimidazo[4,5-d]pyridazine-4-carboxamide trifluoroaceate

The title compound was obtained by using t-butyl 4-[1-(2-butynyl)-4-carbamoyl-6-methyl-7-oxo-6,7-dihydro-1H-dihydroimidazo[4,5-d]pyridazin-2-yl]piperazine-1-carboxylate according to the method described in Example 115(i).

MS m/e (ESI) 330.18(MH⁺—CF₃COOH)

### EXAMPLE 338

### 3-(2-Butynyl)-2-(piperazin-1-yl)-3,5-dihydroimidazo[4,5-c]pyridin-4-one trifluoroacetate

### (a) 2-bromo-1-(2-butynyl)-1H-imidazole-4,5-dicarbonitrile

69.8 g of potassium carbonate and 50 ml N,N-dimethylformamide solution of 74 ml of 1-bromo-2-butyne were added to a 520 ml N,N-dimethylformamide solution of 90.6 g of 2-bromo-1H-imidazole-4,5-dicarbonitrile [CAS No 50847-09-1], and the mixture was heated at 50° C. for eight hours. 1 L of ethyl acetate and 500 ml of water were added to the solution, and the organic layer washed twice with 500 ml of water and then with 500 ml of a saturated sodium chloride solution. The organic layer was dried over magnesium sulfate, and concentrated under reduced pressure. The residue was purified by silica gel column chromatography. Thus, 48.0 g of the title compound was obtained from the fraction eluted with ethyl acetate-hexane (1:4).

¹H-NMR(CDCl₃)

δ 1.87 (t, J=2.3 Hz, 3H) 4.85 (q, J=2.3 Hz, 2H)

### (b) Ethyl 2-bromo-1-(2-butynyl)-5-cyano-1H-imidazole-4-carboxylate

25 ml of concentrated sulfuric acid was added to a 500 ml ethanol solution of 48.0 g of 2-bromo-1-(2-butynyl)-1H-imidazole-4,5-dicarbonitrile, and the mixture was heated under reflux for 110 hours. The reaction solution was cooled to room temperature, and then concentrated under reduced pressure. The residue was dissolved in a mixture consisting of 500 ml of ethyl acetate and 500 ml of water. Potassium hydroxide was then used to adjust the pH of the solution to 8. The aqueous layer was extracted with 500 ml of ethyl acetate, and the organic layers were combined together. The organic layer was dried over magnesium sulfate, and concentrated under reduced pressure. The residue was purified by silica gel column chromatography. Thus, 21.7 g of the title compound was obtained from the fraction eluted with ethyl acetate-hexane (1:3).

¹H-NMR(CDCl₃)

δ 1.43 (t, J=7.0 Hz, 3H) 1.87 (t, J=2.3 Hz, 3H) 4.46 (q, J=7.0 Hz, 2H) 4.85 (q, J=2.3 Hz, 2H)

### (c) t-Butyl 4-[1-(2-butynyl)-5-cyano-4-ethoxycarbonyl-1H-imidazol-2-yl]piperazine-1-carboxylate

25.1 g of the title compound was obtained by using 21.7 g of ethyl 2-bromo-1-(2-butynyl)-5-cyano-1H-imidazole-4-carboxylate according to the method described in Example 115(b).

¹H-NMR(CDCl₃)

δ 1.43 (t, J=7.0 Hz, 3H) 1.49 (s, 9H) 1.87 (t, J=2.3 Hz, 3H) 3.22-3.26 (m, 4H) 3.56-3.61 (m, 4H) 4.44 (q, J=7.0 Hz, 2H) 4.68 (q, J=2.3 Hz, 2H)

### (d) t-Butyl 4-[1-(2-butynyl)-4-carboxy-5-cyano-1H-imidazol-2-yl]piperazine-1-carboxylate

16 ml of a 5N aqueous sodium hydroxide solution was added to a 500 ml ethanol solution of 25.1 g of t-butyl 4-[1-(2-butynyl)-5-cyano-4-ethoxycarbonyl-1H-imidazol-2-yl]piperazine-1-carboxylate, and the mixture was stirred at room temperature for two hours. Then, the solvent was concentrated under reduced pressure. The residue was dissolved in a mixture consisting of IL of ethyl acetate and 500 ml of water. 50 ml of 2N hydrochloric acid was added to the solution. The organic layer washed with 200 ml of a saturated sodium chloride solution, and dried over magnesium sulfate. The organic liquid was concentrated under reduced pressure to give 23.2 g of the title compound.

¹H-NMR(CDCl₃)

δ 1.49 (s, 9H) 1.87 (t, J=2.3 Hz, 3H) 3.22-3.26 (m, 4H) 3.56-3.61 (m, 4H) 4.68 (q, J=2.3 Hz, 2H)

### (e) t-Butyl 4-[1-(2-butynyl)-5-cyano-4-hydroxymethyl-1H-imidazol-2-yl]piperazine-1-carboxylate

6.9 g of triethylamine and then 100 ml tetrahydrofuran solution of 10.19 g of isobutyl chloroformate were added dropwise to 600 ml of tetrahydrofuran containing 22.9 g of t-butyl 4-[1-(2-butynyl)-4-carboxy-5-cyano-1H-imidazol-2-yl]piperazine-1-carboxylate at −10° C. After the precipitate had been removed by filtration, the solution was again cooled to −10° C. A 100 ml aqueous solution of 9.45 g of sodium borohydride was added dropwise to the solution. After one hour, 500 ml of ethyl acetate and 500 ml of water were added to the solution. The pH of the solution was adjusted to 5 using 1 N hydrochloric acid, and then adjusted to 10 using a saturated sodium bicarbonate solution. The organic layer washed successively with 500 ml of water and 500 ml of a saturated sodium chloride solution. The organic layer was dried over magnesium sulfate, and concentrated under reduced pressure. The residue was purified by silica gel column chromatography. Thus, 19.1 g of the title compound was obtained from the fraction eluted with ethyl acetate-hexane (4:1).

¹H-NMR(CDCl₃)

δ 1.48 (s, 9H) 1.84 (t, J=2.3 Hz, 3H) 2.26 (t, J=6.3 Hz, 1H) 3.13-3.17 (m, 4H) 3.53-3.57 (m, 4H) 4.58 (q, J=2.3 Hz, 2H) 4.64 (d, J=6.3 Hz, 2H)

### (f) t-Butyl 4-[1-(2-butynyl)-5-cyano-4-formyl-1H-imidazol-2-yl]piperazine-1-carboxylate

3.28 g of manganese dioxide was added to a 5 ml dichloromethane solution of 1.35 g of t-butyl 4-[1-(2-butynyl)-5-cyano-4-hydroxymethyl-1H-imidazol-2-yl]piperazine-1-carboxylate. The reaction solution was stirred at room temperature for 15 hours, then stirred and heated under reflux for five hours. The solution was filtered, and then concentrated under reduced pressure. The residue was purified by silica gel column chromatography. Thus, 1.11 g of the title compound was obtained from the fraction eluted with ethyl acetate-hexane (2:3).

¹H-NMR(CDCl₃)

δ 1.50 (s, 9H) 1.88 (t, J=2.3 Hz, 3H) 3.24-3.28 (m, 4H) 3.59-3.63 (m, 4H) 4.70 (q, J=2.3 Hz, 2H) 9.87 (s, 1H)

### (g) t-Butyl 4-[1-(2-butynyl)-5-cyano-4-(2-ethoxycarbonylvinyl)-1H-imidazol-2-yl]piperazine-1-carboxylate

0.038 g of sodium hydride was added to a 5 ml tetrahydrofuran solution of 0.243 g of ethyl diethylphosphonoacetate at 5° C. under a nitrogen atmosphere. 0.310 g of t-butyl 4-[1-(2-butynyl)-5-cyano-4-formyl-1H-imidazol-2-yl]piperazine-1-carboxylate dissolved in 5 ml of tetrahydrofuran was added, and the mixture was stirred for 30 minutes. 50 ml of ethyl acetate and 25 ml of 0.1N sodium hydroxide were added to the solution. The organic layer was dried over magnesium sulfate, and concentrated under reduced pressure. The residue was purified by silica gel column chromatography. Thus, 0.380 g of the title compound was obtained from the fraction eluted with ethyl acetate-hexane (3:7).

¹H-NMR(CDCl₃)

δ 1.33 (t, J=7.4 Hz, 3H) 1.50 (s, 9H) 1.86 (t, J=2.3 Hz, 3H) 3.19-3.23 (m, 4H) 3.55-3.59 (m, 4H) 4.25 (q, J=7.4 Hz, 2H) 4.59 (q, J=2.3 Hz, 2H) 6.70 (d, J=15.8 Hz, 1H) 7.50 (d, J=15.8 Hz, 1H)

### (h) t-Butyl 4-[1-(2-butynyl)-5-cyano-4-(2-carboxyvinyl)-1H-imidazol-2-yl]piperazine-1-carboxylate

The title compound was obtained by using t-butyl 4-[1-(2-butynyl)-5-cyano-4-(2-ethoxycarbonylvinyl)-1H-imidazol-2-yl]piperazine-1-carboxylate according to the method described in Example 338(d).

¹H-NMR(CDCl₃)

δ 1.50 (s, 9H) 1.86 (t, J=2.3 Hz, 3H) 3.19-3.23 (m, 4H) 3.55-3.59 (m, 4H) 4.59 (q, J=2.3 Hz, 2H) 6.70 (d, J=15.8 Hz, 1H) 7.50 (d, J=15.8 Hz, 1H)

### (i) t-Butyl 4-[1-(2-butynyl)-5-cyano-4-(2-azidecarbonylvinyl)-1H-imidazol-2-yl]piperazine-1-carboxylate

A mixture consisting of 0.200 g of t-butyl 4-[1-(2-butynyl)-5-cyano-4-(2-carboxyvinyl)-1H-imidazol-2-yl]piperazine-1-carboxylate, 0.073 ml of triethylamine, and a 2 ml t-butanol solution of 0.108 ml of diphenylphosphoryl azide was heated at 50° C. under a nitrogen atmosphere for four hours. 50 ml of ethyl acetate was added to the solution, and the mixture washed with 20 ml of water. The organic layer was dried over magnesium sulfate, and concentrated under reduced pressure. The residue was purified by silica gel column chromatography. Thus, 0.178 g of the title compound was obtained from the fraction eluted with ethyl acetate-hexane (2:3).

¹H-NMR(CDCl₃)

δ 1.48 (s, 9H) 1.86 (t, J=2.2 Hz, 3H) 3.19-3.23 (m, 4H) 3.55-3.59 (m, 4H) 4.59 (q, J=2.2 Hz, 2H) 6.67 (d, J=15.4 Hz, 1H) 7.56 (d, J=15.4 Hz, 1H)

### (j) t-Butyl 4-[4-(2-t-butoxycarbonylaminovinyl)-1-(2-butynyl)-5-cyano-1H-imidazol-2-yl]piperazine-1-carboxylate

A 10 ml t-butanol solution of 0.178 g of t-butyl 4-[1-(2-butynyl)-5-cyano-4-(2-azide carbonylvinyl)-1H-imidazol-2-yl]piperazine-1-carboxylate was heated under reflux under a nitrogen atmosphere for 15 hours. The solvent was concentrated under reduced pressure. The residue was purified by silica gel column chromatography. Thus, 0.169 g of the title compound was obtained from the fraction eluted with ethyl acetate-hexane (9:11).

¹H-NMR(CDCl₃)

δ 1.48 (s, 9H) 1.84 (t, J=2.2 Hz, 3H) 3.16-3.19 (m, 4H) 3.54-3.58 (m, 4H) 4.51 (q, J=2.2 Hz, 2H) 5.83 (d, J=15.0 Hz, 1H) 6.43-6.53 (m, 1H) 7.55-7.66 (m, 1H)

### (k) t-Butyl 4-[4-(2-t-butoxycarbonylaminovinyl)-1-(2-butynyl)-5-carbamoyl-1H-imidazol-2-yl]piperazine-1-carboxylate

The title compound was obtained by using t-butyl 4-[4-(2-t-butoxycarbonylaminovinyl)-1-(2-butynyl)-5-cyano-1H-imidazol-2-yl]piperazine-1-carboxylate according to the method described in Example 332(b).

¹H-NMR(CDCl₃)

δ 1.48 (s, 9H) 1.84 (t, J=2.2 Hz, 3H) 3.21-3.25 (m, 4H) 3.54-3.58 (m, 4H) 4.68 (q, J=2.2 Hz, 2H) 5.90 (br.s, 1H) 6.36 (br.d, J=14.8 Hz, 1H) 6.92 (br.d, J=8.4 Hz, 1H) 7.45 (br.s, 1H) 7.52 (m, 1H)

### (l) 3-(2-Butynyl)-2-(piperazin-1-yl)-3,5-dihydroimidazo[4,5-c]pyridin-4-one trifluoroacetate

0.1 ml of 5N hydrochloric acid was added to a 0.3 ml ethanol solution of 0.0075 g of t-butyl 4-[4-(2-t-butoxycarbonylaminovinyl)-1-(2-butynyl)-5-carbamoyl-1H-imidazol-2-yl]piperazine-1-carboxylate, and the mixture was stirred at room temperature for 15 hours. The solvent was concentrated under reduced pressure. The residue was purified by reverse-phase high performance liquid chromatography (using an acetonitrile-water mobile phase (containing 0.1% trifluoroacetic acid)) to give 0.0043 g of the title compound.

¹H-NMR(CD₃OD)

δ 1.81 (t, J=2.4 Hz, 3H) 3.45-3.48 (m, 4H) 3.62-3.65 (m, 4H) 5.15 (q, J=2.4 Hz, 2H) 6.60 (d, J=7.1 Hz, 1H) 7.18 (d, J=7.1 Hz, 1H)

MS m/e (ESI) 272.32(MH⁺—CF₃COOH)

### EXAMPLE 339

### 3-(2-Butynyl)-5-(2-phenylethyl)-2-(piperazin-1-yl)-3,5-dihydroimidazo[4,5-c]pyridin-4-one trifluoroacetate

### (a) t-Butyl 4-[3-(2-butynyl)-4-oxo-4,5-dihydro-3H-imidazo[4,5-c]pyridin-2-yl]piperazine-1-carboxylate

The title compound was obtained by using 3-(2-butynyl)-2-(piperazin-1-yl)-3,5-dihydroimidazo[4,5-c]pyridin-4-one trifluoroacetate according to the method described in Example 258(a).

¹H-NMR(CDCl₃)

δ 1.49 (s, 9H) 1.83 (t, J=2.3 Hz, 3H) 3.35-3.39 (m, 4H) 3.60-3.64 (m, 4H) 5.07 (q, J=2.3 Hz, 2H) 6.55 (d, J=7.1 Hz, 1H) 6.97 (d, J=7.1 Hz, 1H)

### (b) 3-(2-Butynyl)-5-(2-phenylethyl)-2-(piperazin-1-yl)-3,5-dihydroimidazo[4,5-c]pyridin-4-one trifluoroacetate

The title compound was obtained by using t-butyl 4-[3-(2-butynyl)-4-oxo-4,5-dihydro-3H-imidazo[4,5-c]pyridin-2-yl]piperazine-1-carboxylate and (2-bromoethyl)benzene according to the method described in Example 258(b).

¹H-NMR(CD₃OD)

δ 1.83 (t, J=2.4 Hz, 3H) 3.05 (t, J=7.3 Hz, 2H) 3.45-3.48 (m, 4H) 3.62-3.65 (m, 4H) 4.26 (t, J=7.3 Hz, 2H) 5.18 (q, J=2.4 Hz, 2H) 6.46 (d, J=7.3 Hz, 1H) 7.15 (d, J=7.3 Hz, 1H) 7.16-7.30 (m, 5H)

MS m/e (ESI) 376.36(MH⁺—CF₃COOH)

### EXAMPLE 340

### 3-(2-Butynyl)-5-(2-phenoxyethyl)-2-(piperazin-1-yl)-3,5-dihydroimidazo[4,5-c]pyridin-4-one trifluoroacetate

The title compound was obtained by using t-butyl 4-[3-(2-butynyl)-4-oxo-4,5-dihydro-3H-imidazo[4,5-c]pyridin-2-yl]piperazine-1-carboxylate and 2-bromoethyl phenyl ether according to the method described in Example 258(b).

¹H-NMR(CD₃OD)

δ 1.80 (t, J=2.4 Hz, 3H) 3.45-3.48 (m, 4H) 3.62-3.65 (m, 4H) 4.30 (t, J=5.5 Hz, 2H) 4.44 (t, J=5.5 Hz, 2H) 5.16 (q, J=2.4 Hz, 2H) 6.59 (d, J=6.1 Hz, 1H) 6.87-6.91 (m, 3H) 7.20-7.24 (m, 2H) 7.50 (d, J=6.1 Hz, 1H)

MS m/e (ESI) 392.34(MH⁺—CF₃COOH)

### EXAMPLE 341

### 3-(2-Butynyl)-5-(2-oxo-2-phenylethyl)-2-(piperazin-1-yl)-3,5-dihydroimidazo[4,5-c]pyridin-4-one trifluoroacetate

The title compound was obtained by using t-butyl 4-[3-(2-butynyl)-4-oxo-4,5-dihydro-3H-imidazo[4,5-c]pyridin-2-yl]piperazine-1-carboxylate and 2-bromoacetophenone according to the method described in Example 258(b).

¹H-NMR(CD₃OD)

δ 1.79 (t, J=2.3 Hz, 3H) 3.46-3.50 (m, 4H) 3.64-3.68 (m, 4H) 5.16 (q, J=2.3 Hz, 2H) 5.61 (s, 2H) 6.65 (d, J=7.3 Hz, 1H) 7.37 (d, J=7.3 Hz, 1H) 7.57 (t, J=8.0 Hz, 2H) 7.69 (t, J=8.0 Hz, 1H) 8.10 (d, J=8.0 Hz, 2H)

MS m/e (ESI) 392.34(MH⁺—CF₃COOH)

### EXAMPLE 410

### 7-(2-butvnyl)-1,3-dimethyl-8-(piperazin-1-yl)-3,7-dihydropurine-2,6-dione

### (a) Tert-butyl 4-[7-(2-Butynyl)-1,3-dimethyl-2,6-dioxo-2,3,6,7-tetrahydro-1H-purin-8-yl]piperazine-1-carboxylate

4.9 g of 8-chlorotheophylline and 5 g of potassium carbonate were dissolved in 100 ml of N,N-dimethylformamide, and 2.4 ml of 1-bromo-2-butyne was added thereto. After the mixture was stirred at room temperature overnight, the products were diluted with ethyl acetate and washed with water. The resulting insoluble white solid material was collected by filtration, and washed with ethyl acetate to give 3.8 g of 7-(2-butynyl)-8-chloro-1,3-dimethyl-3,7-dihydropurine-2,6-dione. Then, a 1.8 g aliquot of the yielded 7-(2-butynyl)-8-chloro-1,3-dimethyl-3,7-dihydropurine-2,6-dione and 3.7 g of tert-butyl 1-piperazinecarboxylate were stirred at 150° C. for one hour. The reaction mixture was cooled to room temperature, and then extracted with ethyl acetate. The organic layer washed with water and then with saturated sodium chloride solution, and dried over anhydrous magnesium sulfate. The solvent was distilled off. The residue was purified by silica gel column chromatography. 1.6 g of the title compound was obtained from a fraction eluted with hexane-ethyl acetate (1:4).

¹H-NMR(CDCl₃)

δ 1.49 (s, 9H) 1.82 (t, J=2.4 Hz, 3H) 3.33-3.36 (m, 4H) 3.40 (s, 3H) 3.52 (s, 3H) 3.58-3.61 (m, 4H) 4.88 (q, J=2.4 Hz, 2H)

### (b) 7-(2-Butynyl)-1,3-dimethyl-8-(piperazin-1-yl)-3,7-dihydropurine-2,6-dione

2.5 g of tert-butyl 4-[7-(2-butynyl)-1,3-dimethyl-2,6-dioxo-2,3,6,7-tetrahydro-1H-purin-8-yl]piperazine-1-carboxylate was dissolved in 15 ml of trifluoroacetic acid, and the resulting mixture was stirred at room temperature for 30 minutes. After the solvent was distilled off, the residue was purified by column chromatography using NH-silica gel (silica gel surface-treated with amino groups: NH-DM2035; FUJI SILYSIA CHEMICAL LTD.). 1.6 g of the title compound was obtained from a fraction eluted with ethyl acetate.

¹H-NMR(CDCl₃)

δ 1.82 (t, J=2.4 Hz, 3H) 3.13-3.16 (m, 4H) 3.40 (s, 3H) 3.46-3.48 (m, 4H) 3.52 (s, 3H) 4.87 (q, J=2.4 Hz, 2H)

### TEST EXAMPLE 1

DPPIV-Inhibiting Activity Assay

Porcine kidney-derived DPP-IV was dissolved in a reaction buffer (50 mM Tris-HCl (pH 7.4)/0.1% BSA) at a concentration of 10 mU/ml. After 110 μl of this solution had been combined with 15 μl of an agent, the mixture was incubated at room temperature for 20 minutes. 25 μl of 2 mM Gly-Pro-p-nitroanilide was added (to a final concentration of 0.33 mM) to the solution to initiate the enzyme reaction. The reaction time was 20 minutes. 25 μl of IN phosphoric acid solution was added to the reaction solution to quench the reaction. Absorbance of this solution at 405 nm was determined, and then the inhibition rate for the enzyme reaction was calculated to determine the IC₅₀.

TABLE 1

| Example No. | IC₅₀ (μM) |
|---|---|
| Example 1 | 0.287 |
| Example 4 | 0.211 |
| Example 7 | 0.401 |
| Example 9 | 0.141 |
| Example 12 | 0.183 |
| Example 13 | 0.125 |
| Example 16 | 0.272 |
| Example 20 | 0.152 |
| Example 22 | 0.170 |
| Example 29 | 0.310 |
| Example 53 | 0.0469 |
| Example 64 | 0.126 |
| Example 73 | 0.0334 |
| Example 76 | 0.0865 |
| Example 79 | 0.0357 |
| Example 82 | 0.161 |
| Example 83 | 0.0274 |
| Example 86 | 0.00408 |
| Example 88 | 0.00289 |
| Example 98 | 0.00969 |
| Example 109 | 1.48 |
| Example 119 | 0.154 |
| Example 120 | 0.116 |
| Example 122 | 0.0153 |
| Example 129 | 0.115 |
| Example 142 | 0.0685 |
| Example 146 | 0.0817 |
| Example 159 | 0.0377 |
| Example 229 | 0.00897 |
| Example 230 | 0.000890 |
| Example 234 | 0.00174 |
| Example 235 | 0.00144 |
| Example 238 | 0.00119 |
| Example 243 | 0.00215 |
| Example 248 | 0.00640 |
| Example 266 | 0.00155 |
| Example 267 | 0.00722 |
| Example 297 | 0.00622 |
| Example 311 | 0.0775 |
| Example 341 | 0.00732 |

### TEST EXAMPLE 2

[Experimental Procedure]

The experimental allergic encephalomyelitis (EAE) model has been used as an animal model for multiple sclerosis. The experimental procedure used is described below:

MOG (myeline oligodendrocyte glycoprotein) peptide (MEVGWYRSPFSRVVHLYRNGK) was dissolved to 1 mg/ml in PBS (phosphate buffer solution), and combined with an adjuvant supplemented with 5 mg/ml killed M. tuberculosis H37 RA to prepare an emulsion. Male C57BL/6 mice were immunized using the emulsion. Each mouse was injected subcutaneously at four positions (50 μl each) in the lateral abdominal area. Each mouse also received 30 ng of pertussis toxin intravenously in PBS at the first immunization and two days after that. The mice were evaluated using EAE scores (a score of 0 to 5) as indicated below. Scores were recorded on scorecards. 0, no change; 1, complete flaccidity of tail; 2, weak hind leg gait disturbance; 3, hind leg paralysis; 4, fore leg paralysis; 5, death.

The compounds described below were suspended or dissolved in 0.5% MC (methylcellulose) solution to a desired concentration (30 mg/kg each).

Compound 1X: 7-(2-butynyl)-1,3-dimethyl-8-(piperazin-1-yl)-3,7-dihydropurine-2,6-dione

Compound 2X: 2-[7-(2-butynyl)-1-methyl-6-oxo-8-(piperazin-1-yl)-6,7-dihydro-1H-purin-2-yloxy]benzamide

Compound 3X: 2-(3-aminopiperidin-1-yl)-3-(2-butynyl)-5-methyl-3,5-dihydroimidazo[4,5-d]pyridazin-4-one trifluoroacetate

[Experimental Results]

The effects of the three types of compounds, 1X, 2X, and 3X, on the EAE model were evaluated by their oral administration to mice at a dose of 10 ml/kg/administration, twice a day from post-immunization day 7. The experiment was conducted in duplicate. In both experiments, EAE symptoms were detectable about 12 days after initial immunization, and almost all mice developed the disease 16 days after the immunization in the control group (MC solution-administrated group). When any of the three types of compounds was administered, the degree of development of EAE symptoms was weak compared to in the control group. Thus, the compounds exhibited definite suppressing effects.

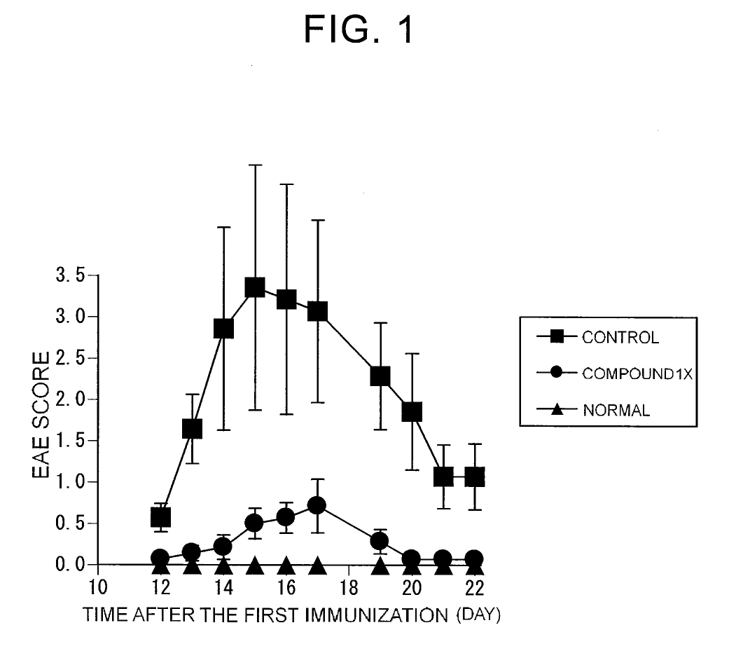

The results of comparing EAE symptoms between compound 1X-administered mice, control group, and normal mice are shown in FIG. 1.

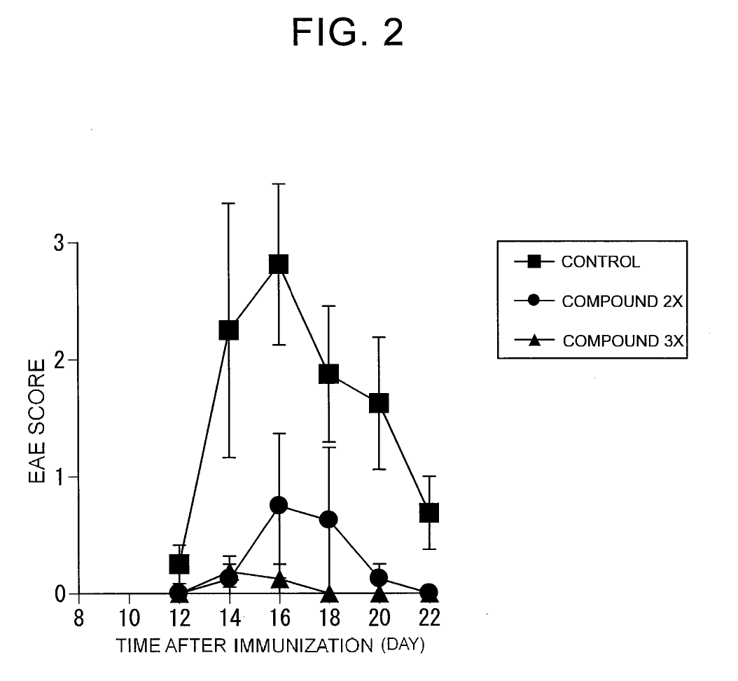

The results of comparing EAE symptoms between compound 2X or 3X-administered mice and control group mice are shown in FIG. 2.

### INDUSTRIAL APPLICABILITY

The condensed imidazole derivatives of the present invention have DPPIV inhibitory action and are thus useful as therapeutic or preventive agents for multiple sclerosis.

1. A method for treating or preventing multiple sclerosis, the method comprising administering to a patient in need thereof a therapeutically effective amount of a compound represented by formula (I), or a pharmaceutically acceptable salt or hydrate thereof, 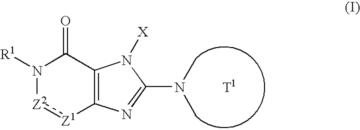
wherein,
T₁ represents a mono- or bicyclic 4- to 12-membered heterocyclic group comprising one or two nitrogen atoms in a ring, which may have substituents;
X represents a C₁₋₆ alkyl group that may have a substituent, a C₂₋₆ alkenyl group that may have a substituent, a C₂₋₆ alkynyl group that may have a substituent, a C₆₋₁₀ aryl group that may have a substituent, a 5- to 10-membered heteroaryl group that may have a substituent, a C₆₋₁₀ aryl C₁₋₆ alkyl group that may have a substituent, or a 5- to 10-membered heteroaryl C₁₋₆ alkyl group that may have a substituent;
in formula (I), the following formula 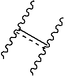
represents a single or double bond;
and when the formula 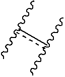
represents a single bond, Z¹ represents a group represented by the formula —NR²—, and Z² represents a carbonyl group;
when the formula 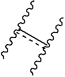
represents a double bond, Z¹ and Z² each independently represent a nitrogen atom or a group represented by the formula —CR²═;
R¹ and R² each independently represent a group represented by the formula -A⁰-A¹-A²
wherein (wherein, A⁰ represents a single bond or a C₁₋₆ alkylene group that may have one to three groups selected from a substituent group B described below;
A¹ represents a single bond, an oxygen atom, a sulfur atom, a sulfinyl group, a sulfonyl group, a carbonyl group, a formula —O—CO—, a formula —CO—O—, a formula —NR^{A}—, a formula —CO—NR^{A}—, a formula —NR^{A}—CO—, a formula —SO₂—NR^{A}—, or a formula —NR^{A}—SO₂—;
A² and R^{A} each independently represent a hydrogen atom, a halogen atom, a cyano group, a guanidino group, a C₁₋₆ alkyl group, a C₃₋₈ cycloalkyl group, a C₃₋₈ cycloalkenyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, a C₆₋₁₀ aryl C₁₋₆ alkyl group, or a C₂₋₇ alkyl carbonyl group;
with the proviso that A² and R^{A} may each independently have one to three moieties selected from substituent group B, substituent group B consisting of:
a hydroxyl group, a mercapto group, a cyano group, a nitro group, a halogen atom, a trifluoromethyl group, a trifluoromethoxy group, an alkylenedioxy group, a C_{—}₁₆ alkyl group that may have a substituent, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a C₁₋₆ alkoxy group, a C₁₋₆ alklthio group;
groups represented by the formulae —SO₂—NR^{B1}—R^{B2}, —NR^{B1}—CO—R^{B2}, and —NR^{B1}—R^{B2},
where R^{B1} and R^{B2} each independently represent a hydrogen atom or a C₆ alkyl group.
a group represented by the formula —CO—R^{B3},
where R^{B3} represents a 4- to 8-membered heterocyclic group.
and groups represented by the formulae —CO—R^{B4}—R^{B5} and —CH₂—CO—R^{B4}—R^{B5}
where R^{B4} represents a single bond, an oxygen atom, or a formula —NRB⁶—; and
R^{B5} and R^{B6} each independently represent a hydrogen atom, a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic C₁₋₆ alkyl group, a C₆₋₁₀ aryl C₁₋₆ alkyl group, or a 5-10-membered heteroaryl C₁₋₆ alkyl group and
when Z² represents the formula —CR²═, R¹ and R² may together form a 5- to 7-membered ring.

2. The method of claim 1, wherein the compound has the formula:

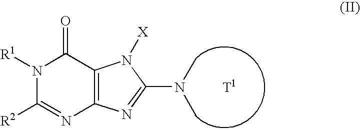

3. The method of claim 1, wherein the compound has the formula:

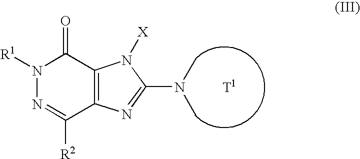

4. The method of claim 1, wherein the compound has the formula:

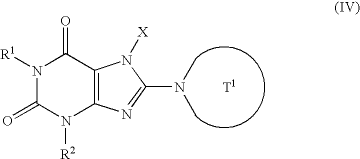

5. The method of claim 1, wherein T¹ is selected from the group consisting of:
an azetidin-1-yl group that may have a substituent:
a pyrrolidine-1-yl group that may have a substituent:
a piperidine-1-yl group that may have a substituent:
an azepan-1-yl group that may have a substituent; and
a group represented by the following formula: 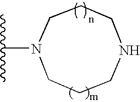
n and m each independently represent zero or one.

6. The method of claim 1, wherein T¹ is selected from the group consisting of:
an azetidin-1-yl group that may have an amino group,
a pyrrolidin-1-yl group that may have an amino group,
a piperidin-1-yl group that may have an amino group;
an azepan-1-yl group that may have an amino group; and
is a group represented by the following formula: 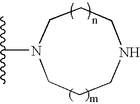
where n and m each independently represent zero or one.

7. The method of claim 1, wherein T¹ is a piperazine-1-yl group or a 3-aminopiperidine-1-yl group.

8. The method of claim 1, wherein T¹ is a piperazine-1-yl group.

9. The method of claim 1, wherein X is a group represented by the formula —X¹—X² where
X¹ represents a single bond or a methylene group that may have a substituent;
X² represents
a C₂₋₆ alkenyl group that may have a substituent,
a C₂₋₆ alkynyl group that may have a substituent, or
a phenyl group that may have a substituent.

10. The method of claim 1, wherein X is a group represented by the formula —X¹¹—X¹² where
X¹¹ represents a single bond or a methylene group;
X¹² represents
a C₂₋₆ alkenyl group,
a C₂₋₆ alkynyl group, or
a phenyl group that may have a substituent.

11. The method of claim 9 or 10, wherein the phenyl group has at position 2 a substituent selected from the group consisting of: a hydroxyl group, a fluorine atom, a chlorine atom, a methyl group, an ethyl group, a fluoromethyl group, a vinyl group, a methoxy group, an ethoxy group, an acetyl group, a cyano group, a formyl group, and a C₂₋₇ alkoxycarbonyl group.

12. The method of claim 1, wherein X is a 3-methyl-2-buten-1-yl group, a 2-butyne-1-yl group, a benzyl group, or a 2-chlorophenyl group.

13. The method of claim 1, wherein X is a 2-butyne-1-yl group.

14. The method of claim 1, wherein R¹ is a hydrogen atom or a group represented by the formula -A¹⁰-A¹¹-A¹²
wherein,
A¹⁰ represents a C₁₋₆ alkylene group that may have one to three moieties groups selected from substituent group C, substituent group C consisting of:
a hydroxyl group, a nitro group, a cyano group, a halogen atom, a C_{—}₆ alkyl group, a C₁₋₆ alkoxy group, a C₁₋₆ alkylthio group, a trifluoromethyl group, a group represented by the formula —NR^{C1}—R^{C2},
where R^{C1} and R^{C2} each independently represent a hydrogen atom or a C₁₋₆ alkyl group,
and groups represented by the formulae —CO—R^{C3}—R^{C4} and —CH₂—CO—R^{C3}—R^{C4}, where R^{C3} represents a single bond, an oxygen atom, or a formula —NR^{C5}—;
and
R^{C4} and R^{C5} each independently represent a hydrogen atom or a C₁₋₆ alkyl group;
A¹¹ represents a single bond, an oxygen atom, a sulfur atom, or a carbonyl group;
A¹² represents
a hydrogen atom,
a C₆₋₁₀ aryl group that may have one to three moieties selected from substituent group C,
a 5- to 10-membered heteroaryl group that may have one to three moieties groups selected from substituent group C,
a 5- to 10-membered heteroaryl C₁₋₆ alkyl group that may have one to three moieties groups selected from substituent group C, or
a C₆₋₁₀ aryl C₁₋₆ alkyl group that may have one to three moieties groups selected from substituent group C.

15. The method of claim 1, wherein
R¹ is
a hydrogen atom,
a C₁₋₆ alkyl group that may have one to three moieties selected from substituent group C, substituent group C consisting of:
a hydroxyl group, a nitro group, a cyano group a halogen atom, a C₁₋₆ alkyl group a C₁₋₆ alkoxy group a C₁₋₆ alkylthio group, a trifluoromethyl group, a group represented by the formula —NR^{C1}—R^{C2},
where R^{C1} and R^{C2} each independently represent a hydrogen atom or a C_{—-6} alkyl group
and groups represented by the formulae —CO—R^{C3}—R^{C4} and —CH₂—CO—R^{C3}—R^{C4}
where R^{C3} represents a single bond an oxygen atom or a formula —NR^{C5}—; and
R^{C4} and R^{C5} each independently represent a hydrogen atom or a C₁₋₆ alkyl group;
a 5- to 10-membered heteroaryl C₁₋₆ alkyl group that may have one to three moieties selected from substituent group C, or
a C₆₋₁₀ aryl C₁₋₆ alkyl group that may have one to three moieties selected from substituent group C.

16. The method of claim 14 or 15 wherein substituent group C consists of a cyano group, a C₁₋₆ alkoxy group, a C₂₋₇ alkoxycarbonyl group, and halogen atom.

17. The method of claim 1, wherein R¹ is a methyl group, a cyanobenzyl group, fluorocyanobenzyl group, a phenethyl group, a 2-methoxyethyl group, or a 4-methoxycarbonylpyridin-2-yl group.

18. The method of claim 1, wherein R¹ is a methyl group or a 2-cyanobenzyl group.

19. The method of claim 1, wherein
R² is
a hydrogen atom,
a cyano group, or
a group represented by the formula -A²¹-A²²
where A²¹ represents
a single bond,
an oxygen atom,
a sulfur atom,
a sulfinyl group,
a sulfonyl group,
a carbonyl group,
a formula —O—CO—,
a formula —CO—O—,
a formula —NR^{A2}—,
a formula —CO—NR^{A2}—,
or a formula —NR^{A2}—CO—,
A²² and R^{A2} each independently represent a hydrogen atom, a cyano group, a C₁₋₆ alkyl group, a C₃₋₈ cycloalkyl group, a C₂₋₆ alkenyl group, a C₂₋₆ alkynyl group, a C₆₋₁₀ aryl group, a 5- to 10-membered heteroaryl group, a 4- to 8-membered heterocyclic group, a 5- to 10-membered heteroaryl C₁₋₆ alkyl group, or a C₆₋₁₀ aryl C₁₋₆ alkyl group;
with the proviso that A²² and R^{A2} each independently may have one to three moieties groups selected from substituent group D, substituent group D consisting of:
a hydroxyl group
a cyano group
a nitro group,
a halogen atom,
a C₁₋₆ alkyl group
a C₁₋₆ alkoxy group
a C₁₋₆ alkylthio group
a trifluoromethyl group.
a group represented by the formula —NR^{D1}—R^{D2}
where R^{D1} and R^{D2} each independently represent a hydrogen atom or a C₁₋₆ alkyl group
a group represented by the formula —CO—R^{D3}
where R^{D3} represents a 4- to 8-membered heterocyclic group, and
a group represented by the formula —CO—R^{D4}—R^{D5}
where R^{D4} represents a single bond, an oxygen atom, or a formula —NR^{D6}—;
R^{D5} and R^{D6} each independently represent a hydrogen atom, a C₃₋₈ cycloalkyl group, or a C₁₋₆ alkyl group.

20. The method of claim 1, wherein
R² is
a hydrogen atom,
a cyano group,
a carboxy group,
a C₂₋₇ alkoxycarbonyl group,
a C₁₋₆ alkyl group,
a group represented by the formula —CONR^{D7}R^{D8}
wherein R^{D7} and R^{D8} each independently represent a hydrogen atom or a C₁₋₆ alkyl group,
or a group represented by the formula -A²³-A²⁴
A²³ represents
an oxygen atom,
a sulfur atom, or
a formula —NR^{A3}—;
A²⁴ and R^{A3} each independently represent
a hydrogen atom,
a C₁₋₆ alkyl group that may have a moiety selected from substituent group, D1, substituent group D1 consisting of:
a carboxy group,
a C₂₋₇ alkoxycarbonyl group
a C₁₋₆ alkyl group,
a group represented by the formula —CONR^{D7}R^{D8}
wherein R^{D7} and R^{D8} each independently represent a hydrogen atom or a C6 alkyl group
a pyrrolidin-1-ylcarbonyl group,
a C₁₋₆ alkyl Iroup, and
C₁₋₆ alkoxy group
a C₃₋₈ cycloalkyl group that may have a moiety selected from substituent group D1,
a C₂₋₆ alkenyl group that may have a moiety selected from substituent group D1,
a C₂₋₆ alkynyl group that may have a moiety selected from substituent group D1,
a phenyl group that may have a moiety selected from substituent group D1, or
a 5- to 10-membered heteroaryl group that may have a moiety selected from substituent group D1.

21. The method of claim 1, wherein
R² is
a hydrogen atom,
a methyl group,
a cyano group,
a C₁₋₆ alkoxy group, or
a group represented by the formula -A²⁵-A²⁶
where A²⁵ represents
an oxygen atom,
a sulfur atom, or
a formula —NR^{A4}—;
A²⁶ and R^{A4} each independently represent
a hydrogen atom,
a C₁₋₆ alkyl group that may have a moiety selected from substituent group D1, substituent grout D1 consisting of:
a carboxy group,
a C₂₋₇ alkoxycarbonyl group,
a C₁₋₆ alkyl group
a group represented by the formula —CONR^{D7}R^{D8}
wherein R^{D7} and R^{D8} each independently represent a hydrogen atom or a C₁₋₆ alkyl group
a pyrrolidin-1-ylcarbonyl groups
a C₁₋₆ alkyl group, and
a C₁₋₆ alkoxy group;
a C₃₋₈ cycloalkyl group that may have a moiety selected from substituent group D1, or
a phenyl group that may have a moiety selected from substituent group D1.

22. The method of claim 1, wherein
R² is
a hydrogen atom,
a cyano group,
a methoxy group,
a carbamoylphenyloxy group, or
a group represented by the following formula: 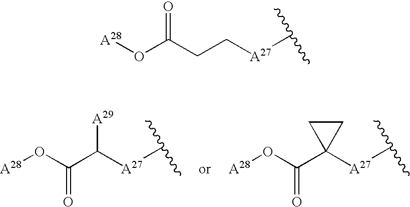
where A²⁷ represents an oxygen atom, a sulfur atom, or —NH—; and
A²⁸ and A²⁹ each independently represent a hydrogen atom or a C₁₋₆ alkyl group.

23. The method of claim 1, wherein R² is a hydrogen atom, a cyano group, or a 2-carbamoylphenyloxy group.

24. The method of claim 1, wherein the compound represented by formula (I) is selected from the group consisting of:
7-(2-butynyl)-1,3-dimethyl-8-(piperazin-1-yl)-3,7-dihydropurine-2,6-dione,
7-(2-butynyl)-2-cyano-1-methyl-8-(piperazin-1-yl)-1,7-dihydropurin-6-one,
3-(2-butynyl)-5-methyl-2-(piperazin-1-yl)-3,5-dihydroimidazo[4,5-d]pyridazin-4-one,
2-(3-aminopiperidin-1-yl)-3-(2-butynyl)-5-methyl-3,5-dihydroimidazo[4,5-d]pyridazin-4-one,
2-[7-(2-butynyl)-1-methyl-6-oxo-8-(piperazin-1-yl)-6,7-dihydro-1H-purin-2-yloxy]benzamide,
7-(2-butynyl)-1-(2-cyanobenzyl)-6-oxo-8-(piperazin-1-yl)-6,7-dihydro-1H-purine-2-carbonitrile, and
2-[3-(2-butynyl)-4-oxo-2-(piperazin-1-yl)-3,4-dihydroimidazo[4,5-d]pyridazin-5-ylmethyl]benzonitrile.

25. The method of claim 1, wherein the compound represented by formula (I) is selected from the group consisting of:
7-(2-butynyl)-2-cyano-1-methyl-8-(piperazin-1-yl)-1,7-dihydropurin-6-one,
3-(2-butynyl)-5-methyl-2-(piperazin-1-yl)-3,5-dihydroimidazo[4,5-d]pyridazin-4-one,
2-(3-aminopiperidin-1-yl)-3-(2-butynyl)-5-methyl-3,5-dihydroimidazo[4,5-d]pyridazin-4-one,
2-[7-(2-butynyl)-1-methyl-6-oxo-8-(piperazin-1-yl)-6,7-dihydro-1H-purin-2-yloxy]benzamide,
7-(2-butynyl)-1-(2-cyanobenzyl)-6-oxo-8-(piperazin-1-yl)-6,7-dihydro-1H-purine-2-carbonitrile, and
2-[3-(2-butynyl)-4-oxo-2-(piperazin-1-yl)-3,4-dihydroimidazo[4,5-d]pyridazin-5-ylmethyl]benzonitrile.
